(19) United States
(12) Patent Application Publication
(10) Pub. No.: US 2017/0087129 A1
(43) Pub. Date: Mar. 30, 2017

(54) COMPOSITIONS AND METHODS FOR THE TREATMENT OF ATHEROSCLEROTIC CARDIOVASCULAR DISEASES WITH PDE4 MODULATORS

(71) Applicant: Celgene Corporation, Summit, NJ (US)

(72) Inventors: Bruce Cronstein, New York, NY (US); Miguel Perez Aso, Valencia (ES)

(21) Appl. No.: 15/311,153

(22) PCT Filed: May 15, 2015
(86) PCT No.: PCT/US15/31116

§ 371 Date: Nov. 14, 2016

Related U.S. Application Data
(60) Provisional application No. 61/994,773, filed on May 16, 2014.

(51) Int. Cl. A61K 31/4035; A61K 9/00

(52) U.S. Cl. CPC A61K 31/4035; A61K 9/0053

(57) ABSTRACT

Provided herein are methods of treating, preventing or managing atherosclerosis by administering a PDE4 modulator. Pharmaceutical compositions, dosage forms, and kits suitable for use in methods are also provided.

### 1. FIELD

Provided herein are compositions and methods for the treatment of atherosclerotic cardiovascular diseases, comprising administering to a subject in need thereof an effective amount of a PDE4 modulator.

### 2. BACKGROUND

Vascular disease, which affects the brain, heart, kidneys, and other various organs, is the leading cause of morbidity and mortality in the United States and other countries. Estimates for 2008 in the U.S. alone are that about 16 million people have atherosclerotic cardiovascular disease (ASCVD). Cardiovascular disease, primarily coronary and cerebrovascular atherosclerosis, caused almost 812,000 deaths in 2008, which is significantly more than those caused by cancer, and 7 time as many injuries were caused by cardiovascular disease. Despite significant medical advances, heart attacks due to atherosclerosis that affects the arteries supplying blood to the heart (coronary artery disease) and stroke due to atherosclerosis that affects the arteries to the brain are responsible for more deaths than any other causes combined.

In rheumatoid arthritis (RA) and psoriasis/psoriatic arthritis, there is a marked increase in risk for development of ASCVD. See, e.g., Chung et al., Arthritis Research & Therapy, 15 (5): R134 (2013). In early RA, the risk factors for development of ASCVD are primarily related to disease activity whereas in late-stage disease conventional risk factors correlate with development of ASCVD. Agents which suppress inflammation in RA, such as Methotrexate or anti-TNF agents, also diminish risk for development of ASCVD although the mechanisms by which they diminish risk (and the underlying causes for the increased risk of ASCVD) have not been fully elucidated. Chung et al., Seminars in Arthritis and Rheumatism, 41(4): 535-44 (2012).

Deposition of cholesterol and lipids in the walls of large arteries are the hallmarks of atherosclerosis and these early lesions are composed of lipid-laden macrophages or foam cells. Over time the composition of these plaques becomes more complex. One mechanism for intrinsic prevention of plaque formation is reverse cholesterol transport from the vessel wall to the liver for disposal into the bile. Macrophages export cholesterol via ABCA1, a membrane transporter, which is believed to be the rate-limiting protein for apolipoprotein-mediated cholesterol efflux from macrophages. Brousseau et al., Journal of Lipid Research, 41(7): 1125-35 (2000); and Lawn et al., The Journal of Clinical Investigation, 104 (8): R25-31 (1999). Thus, induction of ABCA1 expression may provide a novel approach for the treatment of cardiovascular disease. Lin et al., Biochemical and Biophysical Research Communications, 290(2): 663-9 (2002). cAMP has been shown to upregulate ABCA1 expression and increase apoliporotein-mediated cholesterol efflux from macrophages. Lawn et al., The Journal of Clinical Investigation, 104 (8): R25-31 (1999); Bortnick et al., The Journal of Biological Chemistry, 275(37): 28634-40 (2000); Oram et al., The Journal of Biological Chemistry, 275(44):34508-11 (2000); Sakr et al., Biochimica et Biophysica Acta, 1438(1): 85-98 (1999); and Takahashi et al., Biochimica et Biophysica Acta, 1492 (2-3): 385-94 (2000).

Despite progress in the treatment options for ASCVD, ASCVD remains a significant concern, especially for patients with rheumatic and/or autoimmune diseases. Need exists as to an effective treatment, prevention or management of ASCVD.

### 3. SUMMARY

Provided herein are methods for treating, preventing, and/or managing atherosclerosis, comprising administering to a subject in need thereof an effective amount of a PDE4 modulator provided herein. Further provided are compounds being PDE4 modulators for use in methods for treating, preventing, and/or managing atherosclerosis, comprising administering to a subject in need thereof. In some embodiments, the atherosclerosis is ameliorated by modulation of PDE4. In some embodiments, the atherosclsrosis is ameliorated by inhibition of PDE4. In some embodiments, the atherosclerosis is ameliorated by increase of cAMP. In some embodiments, the atherosclerosis is caused by stenosis, thrombosis, aneurysm, or embolus.

In certain embodiments, provided herein are methods for treating, preventing, and/or managing atherosclerotic cardiovascular disease (ASCVD), comprising administering to a subject in need thereof an effective amount of a PDE4 modulator provided herein. In certain embodiments, provided herein is an effective amount of a compound being a PDE4 modulator provided herein for use in methods for treating, preventing, and/or managing atherosclerotic cardiovascular disease (ASVD), wherein the methods comprise administering the compound to a subject in need thereof. In some embodiments, the ASCVD is ameliorated by modulation of PDE4. In some embodiments, the ASCVD is ameliorated by inhibition of PDE4. In some embodiments, the ASCVD is ameliorated by increase of cAMP. In some embodiments, the ASCVD is caused by stenosis, thrombosis, aneurysm, or embolus.

In some embodiments, provided herein are methods for treating symptoms of atherosclerosis. In some embodiments, compounds provided herein being PDE4 modulators are used for treating symptoms of artherosclerosis. Symptoms include, but are not limited to, angina, shortness of breath, arrhythmias, sleep problems, lack of energy, fatigue, sudden weakness, numbness, paralysis, trouble in seeing objects, dizziness, loss of consciousness, severe headache, and claudication.

In some embodiments, the PDE4 modulator is N-(2-(1-(3-ethoxy-4-methoxyphenyl)-2-(methylsulfonyl)ethyl)-1,3-dioxoisoindolin-4-yl)acetamide, or an enantiomer thereof, or a mixture of enantiomers thereof, or a pharmaceutically acceptable salt, solvate, hydrate, clathrate, or prodrug thereof. In one embodiment, the PDE4 modulator is N-(2-(1-(3-ethoxy-4-methoxyphenyl)-2-(methylsulfonyl)ethyl)-1,3-dioxoisoindolin-4-yl)acetamide, or a pharmaceutically acceptable salt, solvate, hydrate, clathrate, or prodrug thereof. In one embodiment, the PDE4 modulator is (S)—N-(2-(1-(3-ethoxy-4-methoxyphenyl)-2-(methylsulfonyl)ethyl)-1,3-dioxoisoindolin-4-yl)acetamide, or a pharmaceutically acceptable salt, solvate, hydrate, clathrate, or prodrug thereof. In another embodiment, the PDE4 modulator is (R)—N-(2-(1-(3-ethoxy-4-methoxyphenyl)-2-(methylsulfonyl)ethyl)-1,3-dioxoisoindolin-4-yl)acetamide, or a pharmaceutically acceptable salt, solvate, hydrate, clathrate, or prodrug thereof.

In certain embodiments, the PDE4 modulator is N-(2-(1-(3-ethoxy-4-methoxyphenyl)-2-(methylsulfonyl)ethyl)-1,3-dioxoisoindolin-4-yl)acetamide, or a pharmaceutically acceptable salt, solvate, hydrate, clathrate, or prodrug thereof. In one embodiment, the PDE4 modulator is (S)—N-(2-(1-(3-ethoxy-4-methoxyphenyl)-2-(methylsulfonyl)ethyl)-1,3-dioxoisoindolin-4-yl)acetamide, or a pharmaceutically acceptable salt, solvate, hydrate, clathrate, or prodrug thereof. In another embodiment, the PDE4 modulator is (R)—N-(2-(1-(3-ethoxy-4-methoxyphenyl)-2-(methylsulfonyl)ethyl)-1,3-dioxoisoindolin-4-yl)acetamide, or a pharmaceutically acceptable salt, solvate, hydrate, clathrate, or prodrug thereof.

In one embodiment, the PDE4 modulator is N-(2-(1-(3-ethoxy-4-methoxyphenyl)-2-(methylsulfonyl)ethyl)-3-oxoisoindolin-4-yl)cyclopropanecarboxamide, or a pharmaceutically acceptable salt, solvate, hydrate, clathrate, or prodrug thereof. In one embodiment, the PDE4 modulator is (S)—N-(2-(1-(3-ethoxy-4-methoxyphenyl)-2-(methylsulfonyl)ethyl)-3-oxoisoindolin-4-yl)cyclopropanecarboxamide, or a pharmaceutically acceptable salt, solvate, hydrate, clathrate, or prodrug thereof. In another embodiment, the PDE4 modulator is (R)—N-(2-(1-(3-ethoxy-4-methoxyphenyl)-2-(methylsulfonyl)ethyl)-3-oxoisoindolin-4-yl)cyclopropanecarboxamide, or a pharmaceutically acceptable salt, solvate, hydrate, clathrate, or prodrug thereof.

In some embodiments, the PDE4 modulator is 3-(3,4-dimethoxy-phenyl)-3-(1-oxo-1,3-dihydro-isoindol-2-yl)-propionamide, or an enantiomer thereof, or a mixture of enantiomers thereof, or a pharmaceutically acceptable salt, solvate, hydrate, clathrate, or prodrug thereof. In one embodiment, the PDE4 modulator is 3-(3,4-dimethoxy-phenyl)-3-(1-oxo-1,3-dihydro-isoindol-2-yl)-propionamide, or a pharmaceutically acceptable salt, solvate, hydrate, clathrate, or prodrug thereof.

### 4. DETAILED DESCRIPTION

### 4.1. BRIEF DESCRIPTION OF FIGURES

FIG. 1 illustrates inhibition of foam cell formation in THP-1 monocytic cell line by Compound A1 (FIG. 1A: THP-1 cells+PMA; FIG. 1B: Acetylated LDL+IFN-γ in THP-1; FIG. 1C: Compound A1+Acetylated LDL+IFN-γ in THP-1 cells; FIG. 1D: Zoom-in of individual cells from FIG. 1B; FIG. 1E: Zoom-in of individual cells from FIG. 1C).

FIG. 2 is a normalized illustration of inhibition of foam cell formation in THP-1 cells by Compound A1.

### 4.2. DEFINITIONS

As used herein and unless otherwise indicated, the term “PDE4 modulators” encompasses small molecule drugs, e.g., small organic molecules which are not peptides, proteins, nucleic acids, oligosaccharides or other macromolecules. In certain embodiments, the compounds inhibit TNF-α production. Compounds may also have an inhibitory effect on LPS induced IL1β and IL12. In some embodiments, the compounds are potent PDE4 inhibitors.

As used herein, the term “Compound A” refers to N-(2-(1-(3-ethoxy-4-methoxyphenyl)-2-(methylsulfonyl)ethyl)-1,3-dioxoisoindolin-4-yl)acetamide, also known as 2-[1-(3-ethoxy-4-methoxyphenyl)-2-methylsulfonylethyl]-4-acetylaminoisoindoline-1,3-dione, which has the following structure:

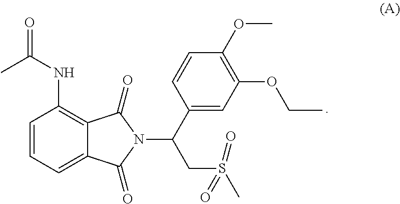 [SMILES: CCOc1cc(C(CS(C)(=O)=O)N2C(=O)c3cccc(NC(C)=O)c3C2=O)ccc1OC]

Compound A also refers to any crystal form or polymorph of N-(2-(1-(3-ethoxy-4-methoxyphenyl)-2-(methylsulfonyl)ethyl)-1,3-dioxoisoindolin-4-yl)acetamide.

As used herein, the term “Compound A1” refers to an enantiomerically pure form of 2-[1-(3-ethoxy-4-methoxyphenyl)-2-methylsulfonylethyl]-4-acetylaminoisoindoline-1,3-dione, also known as Apremilast, and which when dissolved in methanol rotates plane polarized light in the (+) direction. Without being limited by theory, Compound A1 is believed to be (S)—N-(2-(1-(3-ethoxy-4-methoxyphenyl)-2-(methylsulfonyl)ethyl)-1,3-dioxoisoindolin-4-yl)acetamide, which has the following structure:

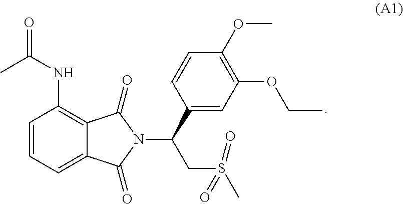 [SMILES: CCOc1cc([C@@H](CS(C)(=O)=O)N2C(=O)c3cccc(NC(C)=O)c3C2=O)ccc1OC]

Compound A1 also refers to any crystal form or polymorph of (S)—N-(2-(1-(3-ethoxy-4-methoxyphenyl)-2-(methylsulfonyl)ethyl)-1,3-dioxoisoindolin-4-yl)acetamide. Examples of crystal form or polymorph of Compound A1 include, but are not limited to, those disclosed in U.S. Pat. No. 7,893,101.

As used herein, the term “Compound A2” refers to an enantiomerically pure form of 2-[1-(3-ethoxy-4-methoxyphenyl)-2-methylsulfonylethyl]-4-acetylaminoisoindoline-1,3-dione, which when dissolved in methanol rotates plane polarized light in the (−) direction. Without being limited by theory, Compound A2 is believed to be (R)—N-(2-(1-(3-ethoxy-4-methoxyphenyl)-2-(methylsulfonyl)ethyl)-1,3-dioxoisoindolin-4-yl)acetamide. Compound A2 also refers to any crystal form or polymorph of (R)—N-(2-(1-(3-ethoxy-4-methoxyphenyl)-2-(methylsulfonyl)ethyl)-1,3-dioxoisoindolin-4-yl)acetamide.

As used herein, the term “Compound B” refers to N-(2-(1-(3-ethoxy-4-methoxyphenyl)-2-(methylsulfonyl)ethyl)-3-oxoisoindolin-4-yl)cyclopropanecarboxamide, also known as cyclopropanecarboxylic acid {2-[(1S)-1-(3-ethoxy-4-methoxy-phenyl)-2-methanesulfonyl-ethyl]-3-oxo-2,3-dihydro-1H-isoindol-4-yl}-amide, which has the following structure:

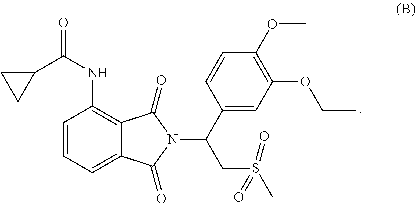 [SMILES: CCOc1cc(C(CS(C)(=O)=O)N2C(=O)c3cccc(NC(=O)C4CC4)c3C2=O)ccc1OC]

Compound B also refers to any crystal form or polymorph of N-(2-(1-(3-ethoxy-4-methoxyphenyl)-2-(methylsulfonyl)ethyl)-3-oxoisoindolin-4-yl)cyclopropanecarboxamide.

As used herein, the term “Compound B1” refers to enantiomerically pure cyclopropanecarboxylic acid {2-[(1S)-1-(3-ethoxy-4-methoxy-phenyl)-2-methanesulfonyl-ethyl]-3-oxo-2,3-dihydro-1H-isoindol-4-yl}-amide. Without being limited by theory, Compound B1 is believed to be (S)—N-(2-(1-(3-ethoxy-4-methoxyphenyl)-2-(methylsulfonyl)ethyl)-3-oxoisoindolin-4-yl)cyclopropanecarboxamide, which has the following structure:

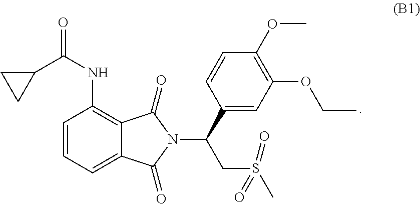 [SMILES: CCOc1cc([C@@H](CS(C)(=O)=O)N2C(=O)c3cccc(NC(=O)C4CC4)c3C2=O)ccc1OC]

Compound B1 also refers to any crystal form or polymorph of (S)—N-(2-(1-(3-ethoxy-4-methoxyphenyl)-2-(methylsulfonyl)ethyl)-3-oxoisoindolin-4-yl)cyclopropanecarboxamide. As used herein, the term “Compound B2” refers to enantiomerically pure cyclopropanecarboxylic acid {2-[(1S)-1-(3-ethoxy-4-methoxy-phenyl)-2-methanesulfonyl-ethyl]-3-oxo-2,3-dihydro-1H-isoindol-4-yl}-amide. Without being limited by theory, Compound B2 is believed to be (R)—N-(2-(1-(3-ethoxy-4-methoxyphenyl)-2-(methylsulfonyl)ethyl)-3-oxoisoindolin-4-yl)cyclopropanecarboxamide. Compound B2 also refers to any crystal form or polymorph of (R)—N-(2-(1-(3-ethoxy-4-methoxyphenyl)-2-(methylsulfonyl)ethyl)-3-oxoisoindolin-4-yl)cyclopropanecarboxamide.

As used herein, the term “Compound C” refers to 3-(3,4-dimethoxyphenyl)-3-(1-oxoisoindolin-2-yl)propanamide, also known as 3-(3,4-dimethoxy-phenyl)-3-(1-oxo-1,3-dihydro-isoindol-2-yl)-propionamide, which has the following chemical structure:

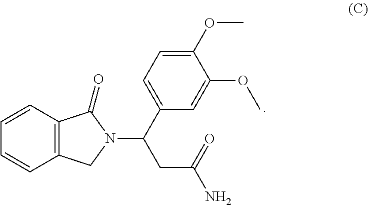 [SMILES: COc1ccc(C(CC(N)=O)N2Cc3ccccc3C2=O)cc1OC]

Compound C also refers to any crystal form or polymorph of 3-(3,4-dimethoxyphenyl)-3-(1-oxoisoindolin-2-yl)propanamide.

As used herein and unless otherwise indicated, the term “pharmaceutically acceptable salt” includes, but is not limited to, salts prepared from pharmaceutically acceptable non-toxic acids or bases including inorganic acids and bases and organic acids and bases. Suitable pharmaceutically acceptable base addition salts include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts.

As used herein and unless otherwise indicated, the term “hydrate” means a compound provided herein or a salt thereof, that further includes a stoichiometric or non-stoichiometric amount of water bound by non-covalent intermolecular forces.

As used herein and unless otherwise indicated, the term “solvate” means a solvate formed from the association of one or more solvent molecules to a compound provided herein. The term “solvate” includes hydrates (e.g., mono-hydrate, dihydrate, trihydrate, tetrahydrate and the like).

As used herein and unless otherwise indicated, the term “polymorph” means solid crystalline forms of a compound provided herein or complex thereof. Different polymorphs of the same compound can exhibit different physical, chemical and/or spectroscopic properties.

As used herein and unless otherwise specified, the term “prodrug” means a derivative of a compound that can hydrolyze, oxidize, or otherwise react under biological conditions (in vitro or in vivo) to provide the compound. Examples of prodrugs include, but are not limited to, derivatives and metabolites of (+)-2-[1-(3-ethoxy-4-methoxyphenyl)-2-methylsulfonylethyl]-4-acetylaminoisoindoline-1,3-dione that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Prodrugs can typically be prepared using well-known methods, such as those described by 1 Burger's Medicinal Chemistry and Drug Discovery, 172-178, 949-982 (Manfred E. Wolff ed., 5th ed. 1995).

As used herein, and unless otherwise specified, the term “enantiomer,” “isomer” or “stereoisomer” encompasses all enantiomerically/stereomerically pure and enantiomerically/stereomerically enriched compounds provided herein.

As used herein, and unless otherwise indicated, the term “stereomerically pure” or “enantiomerically pure” means that a compound comprises one stereoisomer and is substantially free of its counter stereoisomer or enantiomer. For example, a compound is stereomerically or enantiomerically pure, when the compound contains greater than or equal to 80%, 90%, 95%, 98% or 99% of one stereoisomer, and 20%, 10%, 5%, 2%, 1% or less of the counter stereoisomer. “Substantially free of its (−) enantiomer” is encompassed by the term stereomerically pure or enantiomerically pure.

As used herein, and unless otherwise indicated, the term “stereomerically enriched” or “enantiomerically enriched” encompasses racemic mixtures as well as other mixtures of stereoisomers of compounds provided herein (e.g., R/S=30/70, 35/65, 40/60, 45/55, 55/45, 60/40, 65/35 and 70/30).

As used herein, the term “subject” means an animal (e.g., cow, horse, sheep, pig, chicken, turkey, quail, cat, dog, mouse, rat, rabbit, guinea pig, etc.). In certain embodiments, the subject is a mammal such as a non-primate or a primate (e.g., monkey and human). In certain embodiments, the subject is a human. In certain embodiments, the subject is a fetus, embryo, infant, child, adolescent or adult.

As used herein, and unless otherwise specified, the terms “treat,” “treating” and “treatment” contemplate an action that occurs while a patient is suffering from the specified disease or disorder, which reduces the severity or symptoms of the disease or disorder, or retards or slows the progression or symptoms of the disease or disorder.

As used herein, and unless otherwise specified, the term “therapeutically effective amount” of a compound is an amount sufficient to provide a therapeutic benefit in the treatment of a disease or condition, or to delay or minimize one or more symptoms associated with the disease or condition. A therapeutically effective amount of a compound means an amount of therapeutic agent, alone or in combination with other therapies, which provides a therapeutic benefit in the treatment of the disease or condition. The term “therapeutically effective amount” can encompass an amount that improves overall therapy, reduces or avoids symptoms or causes of disease or condition, or enhances the therapeutic efficacy of another therapeutic agent.

### 4.3. PDE4-MODULATORS

Compounds provided herein include racemic, stereomerically pure and stereomerically enriched PDE4 modulators (PDE4 inhibitors), stereomerically and enantiomerically pure compounds that have selective cytokine inhibitory activities, and pharmaceutically acceptable salts, solvates, hydrates, stereoisomers, clathrates, and prodrugs thereof.

In some embodiments, the PDE4 modulator is N-(2-(1-(3-ethoxy-4-methoxyphenyl)-2-(methylsulfonyl)ethyl)-1,3-dioxoisoindolin-4-yl)acetamide, or an enantiomer thereof, or a mixture of enantiomers thereof, or a pharmaceutically acceptable salt, solvate, hydrate, clathrate, or prodrug thereof. In one embodiment, the PDE4 modulator is N-(2-(1-(3-ethoxy-4-methoxyphenyl)-2-(methylsulfonyl)ethyl)-1,3-dioxoisoindolin-4-yl)acetamide (Compound A), or a pharmaceutically acceptable salt, solvate, hydrate, clathrate, or prodrug thereof. In one embodiment, the PDE4 modulator is (S)—N-(2-(1-(3-ethoxy-4-methoxyphenyl)-2-(methylsulfonyl)ethyl)-1,3-dioxoisoindolin-4-yl)acetamide (Compound A1), or a pharmaceutically acceptable salt, solvate, hydrate, clathrate, or prodrug thereof. In another embodiment, the PDE4 modulator is (R)—N-(2-(1-(3-ethoxy-4-methoxyphenyl)-2-(methylsulfonyl)ethyl)-1,3-dioxoisoindolin-4-yl)acetamide (Compound A2), or a pharmaceutically acceptable salt, solvate, hydrate, clathrate, or prodrug thereof.

Compounds A, A1 and A2 can be prepared according to methods disclosed in U.S. Pat. No. 6,962,940, titled “(+)-2-[1-(3-Ethoxy-4-methoxyphenyl)-2-methylsulfonylethyl]-4-acetylaminoisoindoline-1,3-dione: Methods Of Using And Compositions Thereof,” or U.S. Patent Publication No. 2010/0168475, each of which are incorporated herein by reference. Generally, racemic 2-[1-(3-ethoxy-4-methoxyphenyl)-2-methylsulfonylethyl]-4-acetylaminoisoindoline-1,3-dione can be readily prepared using the methods described in U.S. Pat. No. 6,020,358, which is incorporated herein by reference. The corresponding (+) and (−) enantiomers can be isolated from the racemic compound by techniques known in the art. Examples include, but are not limited to, the formation of chiral salts and the use of chiral or high performance liquid chromatography “HPLC” and the formation and crystallization of chiral salts. See, e.g., Jacques, J., et al., Enantiomers, Racemates and Resolutions (Wiley Interscience, New York, 1981); Wilen, S. H., et al., Tetrahedron 33:2725 (1977); Eliel, E. L., Stereochemistry of Carbon Compounds (McGraw Hill, N Y, 1962); and Wilen, S. H., Tables of Resolving Agents and Optical Resolutions p. 268 (E. L. Eliel, Ed., Univ. of Notre Dame Press, Notre Dame, Ind., 1972).

In a specific method, the (+) enantiomer of 2-[1-(3-ethoxy-4-methoxyphenyl)-2-methylsulfonylethyl]-4-acetylaminoisoindoline-1,3-dione is synthesized from 3-acetamidophthalic anhydride and a chiral amino acid salt of (S)-2-(3-ethoxy-4-methoxyphenyl)-1-(methylsulphonyl)-eth-2-ylamine. Chiral amino acid salts of (S)-2-(3 ethoxy-4-methoxyphenyl)-1-(methylsulphonyl)-eth-2-ylamine include, but are not limited to salts formed with the L isomers of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, ornithine, 4-aminobutyric acid, 2-aminoisobutyric acid, 3-aminopropionic acid, ornithine, norleucine, norvaline, hydroxyproline, sarcosine, citrulline, cysteic acid, t-butylglycine, t-butylalanine, phenylglycine, cyclohexylalanine, and N-acetyl-L-leucine. A specific chiral amino acid salt is (S)-2-(3-ethoxy-4-methoxyphenyl)-1-(methylsulphonyl)-eth-2-ylamine N-acetyl-L-leucine salt, which is resolved from 2-(3-ethoxy-4-methoxyphenyl)-1-(methylsulphonyl)-eth-2-ylamine and N-acetyl-L-leucine in methanol.

In some embodiments, the PDE4 modulator is N-(2-(1-(3-ethoxy-4-methoxyphenyl)-2-(methylsulfonyl)ethyl)-3-oxoisoindolin-4-yl)cyclopropanecarboxamide, or an enantiomer thereof, or a mixture of enantiomers thereof, or a pharmaceutically acceptable salt, solvate, hydrate, clathrate, or prodrug thereof. In one embodiment, the PDE4 modulator is N-(2-(1-(3-ethoxy-4-methoxyphenyl)-2-(methylsulfonyl)ethyl)-3-oxoisoindolin-4-yl)cyclopropanecarboxamide (Compound B), or a pharmaceutically acceptable salt, solvate, hydrate, clathrate, or prodrug thereof. In one embodiment, the PDE4 modulator is (S)—N-(2-(1-(3-ethoxy-4-methoxyphenyl)-2-(methylsulfonyl)ethyl)-3-oxoisoindolin-4-yl)cyclopropanecarboxamide (Compound B1), or a pharmaceutically acceptable salt, solvate, hydrate, clathrate, or prodrug thereof. In another embodiment, the PDE4 modulator is (R)—N-(2-(1-(3-ethoxy-4-methoxyphenyl)-2-(methylsulfonyl)ethyl)-3-oxoisoindolin-4-yl)cyclopropanecarboxamide (Compound B2), or a pharmaceutically acceptable salt, solvate, hydrate, clathrate, or prodrug thereof.

In some embodiments, the PDE4 modulator is 3-(3,4-dimethoxy-phenyl)-3-(1-oxo-1,3-dihydro-isoindol-2-yl)-propionamide, or an enantiomer thereof, or a mixture of enantiomers thereof, or a pharmaceutically acceptable salt, solvate, hydrate, clathrate, or prodrug thereof. In one embodiment, the PDE4 modulator is 3-(3,4-dimethoxy-phenyl)-3-(1-oxo-1,3-dihydro-isoindol-2-yl)-propionamide (Compound C), or a pharmaceutically acceptable salt, solvate, hydrate, clathrate, or prodrug thereof.

Examples of PDE4 modulators provided herein include, but are not limited to, the cyclic imides disclosed in U.S. Pat. Nos. 5,605,914 and 5,463,063; the cycloalkyl amides and cycloalkyl nitriles of U.S. Pat. Nos. 5,728,844, 5,728,845, 5,968,945, 6,180,644 and 6,518,281; the aryl amides (for example, an embodiment being N-benzoyl-3-amino-3-(3′,4′-dimethoxyphenyl)-propanamide) of U.S. Pat. Nos. 5,801,195, 5,736,570, 6,046,221 and 6,284,780; the imide/amide ethers and alcohols (for example, 3-phthalimido-3-(3′,4′-dimethoxyphenyl)propan-1-ol) disclosed in U.S. Pat. No. 5,703,098; the succinimides and maleimides (for example methyl 3-(3′,4′,5′6′-petrahydrophthalimdo)-3-(3″,4″-dimethoxyphenyl)propionate) disclosed in U.S. Pat. No. 5,658,940; imido and amido substituted alkanohydroxamic acids disclosed in U.S. Pat. No. 6,214,857 and WO 99/06041; substituted phenethylsulfones disclosed in U.S. Pat. Nos. 6,011,050 and 6,020,358; fluoroalkoxy-substituted 1,3-dihydro-isoindolyl compounds disclosed in U.S. Pat. No. 7,173,058; substituted imides (for example, 2-phthalimido-3-(3′,4′-dimethoxyphenyl) propane) disclosed in U.S. Pat. No. 6,429,221; substituted 1,3,4-oxadiazoles (for example, 2-[1-(3-cyclopentyloxy-4-methoxyphenyl)-2-(1,3,4-oxadiazole-2-yl)ethyl]-5-methylisoindoline-1,3-dione) disclosed in U.S. Pat. No. 6,326,388; cyano and carboxy derivatives of substituted styrenes (for example, 3,3-bis-(3,4-dimethoxyphenyl) acrylonitrile) disclosed in U.S. Pat. Nos. 5,929,117, 6,130,226, 6,262,101 and 6,479,554; isoindoline-1-one and isoindoline-1,3-dione substituted in the 2-position with an α-(3,4-disubstituted phenyl)alkyl group and in the 4- and/or 5-position with a nitrogen-containing group disclosed in WO 01/34606 and U.S. Pat. No. 6,667,316, for example, cyclopropyl-N-{2-[1-(3-ethoxy-4-methoxyphenyl)-2-(methylsulfonyl)ethyl]-3-oxoisoindolin-4-yl}carboxamide, cyclopropyl-N-{2-[1 (S)-(3-ethoxy-4-methoxyphenyl)-2-(methylsulfonyl)ethyl]-3-oxoisoindolin-4-yl}carboxamide, and cyclopropyl-N-{2-[1(R)-(3-ethoxy-4-methoxyphenyl)-2-(methylsulfonyl)ethyl]-3-oxoisoindolin-4-yl}carboxamide; and imido and amido substituted acylhydroxamic acids (for example, (3-(1,3-dioxoisoindoline-2-yl)-3-(3-ethoxy-4-methoxyphenyl) propanoylamino) propanoate disclosed in WO 01/45702 and U.S. Pat. No. 6,699,899. Other PDE4 modulators include diphenylethylene compounds disclosed in U.S. Pat. No. 7,312,241, the contents of which are incorporated by reference herein in their entirety. Other PDE4 modulators include isoindoline compounds disclosed in U.S. patent publication no. 2006/0025457A1, published Feb. 2, 2006 and U.S. Pat. No. 7,244,759. Other specific PDE4 modulators include 2-[1-(3-ethoxy-4-methoxyphenyl)-2-methylsulfonylethyl]-4-acetylaminoisoindoline-1,3-dione, and stereoisomers thereof. (+)-2-[1-(3-ethoxy-4-methoxyphenyl)-2-methylsulfonylethyl]-4-acetylaminoisoindoline-1,3-dione was disclosed in WO 03/080049. The entireties of each of the patents and patent applications identified herein are incorporated herein by reference.

Additional PDE4 modulators belong to a family of synthesized chemical compounds of which typical embodiments include 3-(1,3-dioxobenzo-[f]isoindol-2-yl)-3-(3-cyclopentyloxy-4-methoxyphenyl)propionamide and 3-(1,3-dioxo-4-azaisoindol-2-yl)-3-(3,4-dimethoxyphenyl)-propionamide.

Other PDE4 modulators belong to a class of non-polypeptide cyclic amides disclosed in U.S. Pat. Nos. 5,698,579, 5,877,200, 6,075,041 and 6,200,987, and WO 95/01348, each of which is incorporated herein by reference. Representative cyclic amides include compounds of the formula:

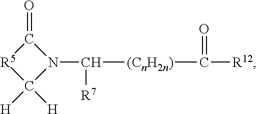

or a pharmaceutically acceptable salt, solvate, hydrate, clathrate, stereoisomer, or prodrug thereof, wherein:
n has a value of 1, 2, or 3;
R⁵ is o-phenylene, unsubstituted or substituted with 1 to 4 substituents each selected independently from the group consisting of nitro, cyano, trifluoromethyl, carbethoxy, carbomethoxy, carbopropoxy, acetyl, carbamoyl, acetoxy, carboxy, hydroxy, amino, alkylamino, dialkylamino, acylamino, alkyl of 1 to 10 carbon atoms, alkyl of 1 to 10 carbon atoms, or halo;
R⁷ is (i) phenyl or phenyl substituted with one or more substituents each selected independently of the other from the group consisting of nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo; (ii) benzyl unsubstituted or substituted with 1 to 3 substituents selected from the group consisting of nitro, cyano, trifluoromethyl, carbothoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo; (iii) naphthyl; or (iv) benzyloxy;
R¹² is —OH, alkoxy of 1 to 12 carbon atoms, or

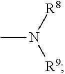

R⁸ is hydrogen or alkyl of 1 to 10 carbon atoms; and
R⁹ is hydrogen, alkyl of 1 to 10 carbon atoms, —COR¹⁰, or —SO₂R¹⁰,
R¹⁰ is hydrogen, alkyl of 1 to 10 carbon atoms, or phenyl.

In some embodiments, the PDE4 modulator is selected from:
3-phenyl-2-(1-oxoisoindolin-2-yl)propionic acid;
3-phenyl-2-(1-oxoisoindolin-2-yl)propionamide;
3-phenyl-3-(1-oxoisoindolin-2-yl)propionic acid;
3-phenyl-3-(1-oxoisoindolin-2-yl)propionamide;
3-(4-methoxyphenyl)-3-(1-oxisoindolin-yl)propionic acid;
3-(4-methoxyphenyl)-3-(1-oxisoindolin-yl)propionamide;
3-(3,4-dimethoxyphenyl)-3-(1-oxisoindolin-2-yl)propionic acid;
3-(3,4-dimethoxy-phenyl)-3-(1-oxo-1,3-dihydroisoindol-2-yl)propionamide;
3-(3,4-dimethoxyphenyl)-3-(1-oxisoindolin-2-yl)propionamide;
3-(3,4-diethoxyphenyl)-3-(1-oxoisoindolin-yl)propionic acid;
methyl 3-(1-oxoisoindolin-2-yl)-3-(3-ethoxy-4-methoxyphenyl)propionate;
3-(1-oxoisoindolin-2-yl)-3-(3-ethoxy-4-methoxyphenyl)propionic acid;
3-(1-oxoisoindolin-2-yl)-3-(3-propoxy-4-methoxyphenyl)propionic acid;
3-(1-oxoisoindolin-2-yl)-3-(3-butoxy-4-methoxyphenyl)propionic acid;
3-(1-oxoisoindolin-2-yl)-3-(3-propoxy-4-methoxyphenyl)propionamide;
3-(1-oxoisoindolin-2-yl)-3-(3-butoxy-4-methoxyphenyl)propionamide;
methyl 3-(1-oxoisoindolin-2-yl)-3-(3-butoxy-4-methoxyphenyl)propionate; and
methyl 3-(1-oxoisoindolin-2-yl)-3-(3-propoxy-4-methoxyphenyl)propionate
   or a pharmaceutically acceptable salt, solvate, hydrate, clathrate, stereoisomer, or prodrug thereof.

Other representative cyclic amides include compounds of the formula:

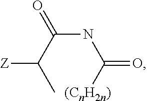

or a pharmaceutically acceptable salt, solvate, hydrate, clathrate, stereoisomer, or prodrug thereof,
wherein

### Z is:

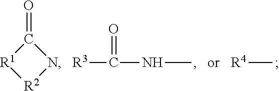

R¹ is the divalent residue of (i) 3,4-pyridine, (ii) pyrrolidine, (iii) imidizole, (iv) naphthalene, (v) thiophene, or (vi) a straight or branched alkane of 2 to 6 carbon atoms, unsubstituted or substituted with phenyl or phenyl substituted with nitro, cyano, trifluoromethyl, carbethoxy, carbomethoxy, carbopropoxy, acetyl, carbamyl, acetoxy, carboxy, hydroxy, amino, alkyl of 1 to 10 carbon atoms, alkoxy of 1 to 10 carbon atoms, or halo, wherein the divalent bonds of said residue are on vicinal ring carbon atoms;
R² is —CO— or —SO₂—;
R³ is (i) phenyl substituted with 1 to 3 substituents each selected independently from nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, or halo; (ii) pyridyl; (iii) pyrrolyl; (iv) imidazolyl; (iv) naphthyl; (vi) thienyl; (vii) quinolyl; (viii) furyl; or (ix) indolyl;
R⁴ is alanyl, arginyl, glycyl, phenylglycyl, histidyl, leucyl, isoleucyl, lysyl, methionyl, prolyl, sarcosyl, seryl, homoseryl, threonyl, thyronyl, tyrosyl, valyl, benzimidol-2-yl, benzoxazol-2-yl, phenylsulfonyl, methylphenylsulfonyl, or phenylcarbamoyl; and
n has a value of 1, 2, or 3.

Other representative cyclic amides include compounds of the formula:

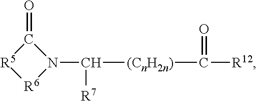

or a pharmaceutically acceptable salt, solvate, hydrate, clathrate, stereoisomer, or prodrug thereof, wherein:
R⁵ is (i) o-phenylene, unsubstituted or substituted with 1 to 4 substituents each selected independently from nitro, cyano, trifluoromethyl, carbethoxy, carbomethoxy, carbopropoxy, acetyl, carbamoyl, acetoxy, carboxy, hydroxy, amino, alkylamino, dialkylamino, acylamino, alkyl of 1 to 10 carbon atoms, alkoxy of 1 to 10 carbon atoms, or halo; or (ii) the divalent residue of pyridine, pyrrolidine, imidizole, naphthalene, or thiophene, wherein the divalent bonds are on vicinal ring carbon atoms;
R⁶ is —CO—, —CH₂—, or —SO₂—;
R⁷ is (i) hydrogen if R⁶ is —SO₂—; (ii) straight, branched, or cyclic alkyl of 1 to 12 carbon atoms; (iii) pyridyl; (iv) phenyl or phenyl substituted with one or more substituents each selected independently of the other from nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, or halo; (v) alkyl of 1 to 10 carbon atoms; (vi) benzyl unsubstituted or substituted with 1 to 3 substituents selected from the group consisting of nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, or halo; (vii) naphthyl; (viii) benzyloxy; or (ix) imidazol-4-yl methyl;
R¹² is —OH, alkoxy of 1 to 12 carbon atoms, or

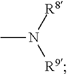

n has a value of 0, 1, 2, or 3;
R^{8′} is hydrogen or alkyl of 1 to 10 carbon atoms; and
R^{9′} is hydrogen, alkyl of 1 to 10 carbon atoms, —COR¹⁰, or —SO₂R¹⁰ in which R¹⁰ is hydrogen, alkyl of 1 to 10 carbon atoms, or phenyl.

Other representative imides include compounds of the formula:

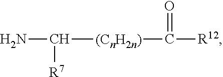

or a pharmaceutically acceptable salt, solvate, hydrate, clathrate, stereoisomer, or prodrug thereof, wherein:
R⁷ is (i) straight, branched, or cyclic alkyl of 1 to 12 carbon atoms; (ii) pyridyl; (iii) phenyl or phenyl substituted with one or more substituents each selected independently of the other from nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, or halo; (iv) benzyl unsubstituted or substituted with one to three substituents selected from the group consisting of nitro, cyano, trifluoromethyl, carbethoxy, carbomethoxy, carbopropoxy, acetyl, carbamoyl, acetoxy, carboxy, hydroxy, amino, alkyl of 1 to 4 carbon atoms, alkoxy of 1 to 4 carbon atoms, or halo; (v) naphthyl; (vi) benzyloxy; or (vii) imidazol-4-ylmethyl;
R¹² is —OH, alkoxy of 1 to 12 carbon atoms, —O—CH₂-pyridyl, —O-benzyl or

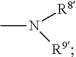

where n has a value of 0, 1, 2, or 3;
R^{8′} is hydrogen or alkyl of 1 to 10 carbon atoms;
R^{9′} is hydrogen, alkyl of 1 to 10 carbon atoms, —CH₂-pyridyl, benzyl, —COR¹⁰, or —SO₂R¹⁰; and
R¹⁰ is hydrogen, alkyl of 1 to 4 carbon atoms, or phenyl.

Other PDE4 modulators include the imido and amido substituted alkanohydroxamic acids disclosed in WO 99/06041 and U.S. Pat. No. 6,214,857, each of which is incorporated herein by reference. Examples of such compounds include, but are not limited to:

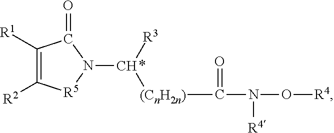

or a pharmaceutically acceptable salt, solvate, hydrate, clathrate, stereoisomer, or prodrug thereof, wherein:
each of R¹ and R², when taken independently of each other, is hydrogen, lower alkyl; or R¹ and R², when taken together with the depicted carbon atoms to which each is bound, is o-phenylene, o-naphthylene, or cyclohexene-1,2-diyl, unsubstituted or substituted with 1 to 4 substituents each selected independently from the group consisting of nitro, cyano, trifluoromethyl, carbethoxy, carbomethoxy, carbopropoxy, acetyl, carbamoyl, acetoxy, carboxy, hydroxy, amino, alkylamino, dialkylamino, acylamino, alkyl of 1 to 10 carbon atoms, alkoxy of 1 to 10 carbon atoms, and halo;
R³ is phenyl substituted with from one to four substituents selected from the group consisting of nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, alkylthio of 1 to 10 carbon atoms, benzyloxy, cycloalkoxy of 3 to 6 carbon atoms, C₄-C₆-cycloalkylidenemethyl, C₃-C₁₀-alkylidenemethyl, indanyloxy, and halo;
R⁴ is hydrogen, alkyl of 1 to 6 carbon atoms, phenyl, or benzyl;
R^{4′} is hydrogen or alkyl of 1 to 6 carbon atoms;
R⁵ is —CH₂—, —CH₂—CO—, —SO₂—, —S—, or —NHCO—; and
n has a value of 0, 1, or 2.

In some embodiments, the PDE4 modulator is the following compound, or a pharmaceutically acceptable salt, solvate, hydrate, clathrate, stereoisomer, or prodrug thereof:
3-(3-ethoxy-4-methoxyphenyl)-N-hydroxy-3-(1-oxoisoindolinyl)propionamide;
3-(3-ethoxy-4-methoxyphenyl)-N-methoxy-3-(1-oxoisoindolinyl)propionamide;
N-benzyloxy-3-(3-ethoxy-4-methoxyphenyl)-3-phthalimidopropionamide;
N-benzyloxy-3-(3-ethoxy-4-methoxyphenyl)-3-(3-nitrophthalimido)propionamide;
N-benzyloxy-3-(3-ethoxy-4-methoxyphenyl)-3-(1-oxoisoindolinyl)propionamide;
3-(3-ethoxy-4-methoxyphenyl)-N-hydroxy-3-phthalimidopropionamide;
N-hydroxy-3-(3,4-dimethoxyphenyl)-3-phthalimidopropionamide;
3-(3-ethoxy-4-methoxyphenyl)-N-hydroxy-3-(3-nitrophthalimido)propionamide;
N-hydroxy-3-(3,4-dimethoxyphenyl)-3-(1-oxoisoindolinyl)propionamide;
3-(3-ethoxy-4-methoxyphenyl)-N-hydroxy-3-(4-methyl-phthalimido)propionamide;
3-(3-cyclopentyloxy-4-methoxyphenyl)-N-hydroxy-3-phthalimidopropionamide;
3-(3-ethoxy-4-methoxyphenyl)-N-hydroxy-3-(1,3-dioxo-2,3-dihydro-1H-benzo[f]isoindol-2-yl)propionamide;
N-hydroxy-3-{3-(2-propoxy)-4-methoxyphenyl}-3-phthalimidopropionamide;
3-(3-ethoxy-4-methoxyphenyl)-3-(3,6-difluorophthalimido)-N-hydroxypropionamide;
3-(4-aminophthalimido)-3-(3-ethoxy-4-methoxyphenyl)-N-hydroxypropionamide;
3-(3-aminophthalimido)-3-(3-ethoxy-4-methoxyphenyl)-N-hydroxypropionamide;
3-(3-acetoamidophthalimido)-3-(3-ethoxy-4-methoxyphenyl)-N-hydroxypropionamide;
N-hydroxy-3-(3,4-dimethoxyphenyl)-3-(1-oxoisoindolinyl)propionamide;
3-(3-cyclopentyloxy-4-methoxyphenyl)-N-hydroxy-3-(1-oxoisoindolinyl) propionamide; or
N-benzyloxy-3-(3-ethoxy-4-methoxyphenyl)-3-(3-nitrophthalimido)propionamide.

Other PDE4 modulators include the substituted phenethylsulfones substituted on the phenyl group with a oxoisoindine group. Examples of such compounds include, but are not limited to, those disclosed in U.S. Pat. No. 6,020,358, which is incorporated herein by reference, which include the following:

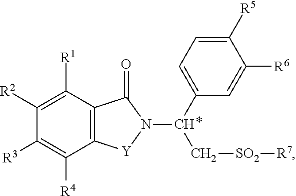

or a pharmaceutically acceptable salt, solvate, hydrate, clathrate, stereoisomer, or prodrug thereof, wherein:
the carbon atom designated * constitutes a center of chirality;
Y is C═O, CH₂, SO₂, or CH₂C═O; each of R¹, R², R³, and R⁴, independently of the others, is hydrogen, halo, alkyl of 1 to 4 carbon atoms, alkoxy of 1 to 4 carbon atoms, nitro, cyano, hydroxy, or —NR⁸R⁹; or any two of R¹, R², R³, and R⁴ on adjacent carbon atoms, together with the depicted phenylene ring are naphthylidene;
each of R⁵ and R⁶, independently of the other, is hydrogen, alkyl of 1 to 4 carbon atoms, alkoxy of 1 to 4 carbon atoms, cyano, or cycloalkoxy of up to 18 carbon atoms;
R⁷ is hydroxy, alkyl of 1 to 8 carbon atoms, phenyl, benzyl, or NR^{8′}R^{9′};
each of R⁸ and R⁹ taken independently of the other is hydrogen, alkyl of 1 to 8 carbon atoms, phenyl, or benzyl; or one of R⁸ and R⁹ is hydrogen and the other is —COR¹⁰ or —SO₂R¹⁰; or R⁸ and
R⁹ taken together are tetramethylene, pentamethylene, hexamethylene, or —CH₂CH₂X¹CH₂CH₂— in which X¹ is —O—, —S— or —NH—; and
each of R^{8′} and R^{9′} taken independently of the other is hydrogen, alkyl of 1 to 8 carbon atoms, phenyl, or benzyl; or one of R^{8′} and R^{9′} is hydrogen and the other is —COR^{10′} or —SO₂R^{10′}; or R^{8′} and R^{9′} taken together are tetramethylene, pentamethylene, hexamethylene, or —CH₂CH₂X²CH₂CH₂— in which X² is —O—, —S—, or —NH—.

It will be appreciated that while for convenience the above compounds are identified as phenethylsulfones, they include sulfonamides when R⁷ is NR^{8′}R^{9′}.

In some embodiments, the compounds are those in which Y is C═O or CH₂.

In other embodiments, the compounds are those in which each of R¹, R², R³, and R⁴ independently of the others, is hydrogen, halo, methyl, ethyl, methoxy, ethoxy, nitro, cyano, hydroxy, or —NR⁸R⁹ in which each of R⁸ and R⁹ taken independently of the other is hydrogen or methyl or one of R⁸ and R⁹ is hydrogen and the other is —COCH₃.

In other embodiments, the compounds are those in which one of R¹, R², R³, and R⁴ is —NH₂ and the remaining of R¹, R², R³, and R⁴ are hydrogen.

In other embodiments, the compounds are those in which one of R¹, R², R³, and R⁴ is —NHCOCH₃ and the remaining of R¹, R², R³, and R⁴ are hydrogen.

In other embodiments, the compounds are those in which one of R¹, R², R³, and R⁴ is —N(CH₃)₂ and the remaining of R¹, R², R³, and R⁴ are hydrogen.

In other embodiments, the compounds are those in which one of R¹, R², R³, and R⁴ is methyl and the remaining of R¹, R², R³, and R⁴ are hydrogen.

In other embodiments, the compounds are those in which one of R¹, R², R³, and R⁴ is fluoro and the remaining of R¹, R², R³, and R⁴ are hydrogen.

In other embodiments, the compounds are those in which each of R⁵ and R⁶, independently of the other, is hydrogen, methyl, ethyl, propyl, methoxy, ethoxy, propoxy, cyclopentoxy, or cyclohexoxy.

In other embodiments, the compounds are those in which R⁵ is methoxy and R⁶ is monocycloalkoxy, polycycloalkoxy, and benzocycloalkoxy.

In other embodiments, the compounds are those in which R⁵ is methoxy and R⁶ is ethoxy.

In other embodiments, the compounds are those in which R⁷ is hydroxy, methyl, ethyl, phenyl, benzyl, or NR^{8′}R^{9′} in which each of R^{8′} and R^{9′} taken independently of the other is hydrogen or methyl.

In other embodiments, the compounds are those in which R⁷ is methyl, ethyl, phenyl, benzyl or NR^{8′}R^{9′} in which each of R^{8′} and R^{9′} taken independently of the other is hydrogen or methyl.

In other embodiments, the compounds are those in which R⁷ is methyl.

In other embodiments, the compounds are those in which R⁷ is NR⁸R^{9′} in which each of R^{8′} and R^{9′} taken independently of the other is hydrogen or methyl.

Other PDE4 modulators include fluoroalkoxy-substituted 1,3-dihydro-isoindolyl compounds disclosed in U.S. Pat. No. 7,173,058, which is incorporated herein by reference. Representative compounds include those of formula:

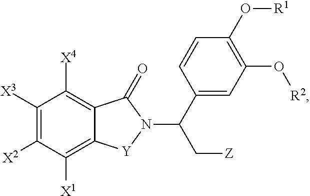

or a pharmaceutically acceptable salt, solvate, hydrate, clathrate, stereoisomer, or prodrug thereof, wherein:
Y is —C(O)—, —CH₂, —CH₂C(O)—, —C(O)CH₂—, or SO₂;
Z is —H, —C(O)R³, —(C₀₋₁-alkyl)-SO₂—(C₁₋₄-alkyl), —C₁₋₈-alkyl, —CH₂OH, CH₂(O)(C₁₋₈-alkyl) or —CN;
R¹ and R² are each independently —CHF₂, —C₁₋₈-alkyl, —C₃₋₁₈-cycloalkyl, or —(C₁₋₁₀-alkyl)(C₃₋₁₈-cycloalkyl), and at least one of R₁ and R₂ is CHF₂;
R³ is —NR⁴R⁵, -alkyl, —OH, —O-alkyl, phenyl, benzyl, substituted phenyl, or substituted benzyl;
R⁴ and R⁵ are each independently —H, —C₁₋₈-alkyl, —OH, or —OC(O)R⁶;
R⁶ is —C₁₋₈-alkyl, -amino(C₁₋₈-alkyl), -phenyl, -benzyl, or -aryl;
X¹, X², X³, and X⁴ are each independently —H, -halogen, -nitro, —NH₂, —CF₃, —C₁₋₆-alkyl, —(C₀₋₄-alkyl)-(C₃₋₆-cycloalkyl), (C₀₋₄-alkyl)-NR⁷R⁸, (C₀₋₄-alkyl)-N(H)C(O)—(R), (C₀₋₄-alkyl)-N(H)C(O)N(R⁷R⁸), (C₀₋₄-alkyl)-N(H)C(O)O(R⁷R⁸), (C₀₋₄-alkyl)-OR⁸, (C₀₋₄-alkyl)-imidazolyl, (C₀₋₄-alkyl)-pyrrolyl, (C₀₋₄-alkyl)-oxadiazolyl, or (C₀₋₄-alkyl)-triazolyl, or two of X¹, X², X³, and X⁴ may be joined together to form a cycloalkyl or heterocycloalkyl ring, (e.g., X¹ and X², X² and X³, X³ and X⁴, X¹ and X³, X² and X⁴, or X¹ and X⁴ may form a 3, 4, 5, 6, or 7 membered ring which may be aromatic, thereby forming a bicyclic system with the isoindolyl ring); and
R⁷ and R⁸ are each independently H, C₁₋₉-alkyl, C₃₋₆-cycloalkyl, (C₁₋₆-alkyl)-(C₃₋₆-cycloalkyl), (C₁₋₆-alkyl)-N(R⁷R⁸), (C₁₋₆-alkyl)-OR⁸, phenyl, benzyl, or aryl.

Other PDE4 modulators include the enantiomerically pure compounds disclosed in U.S. Pat. No. 6,962,940; international patent publication nos. WO 2003/080048 and WO 2003/080049; U.S. Pat. No. 7,312,241 to G. Muller et al.; and U.S. patent publication no. 2004/0167199A1, published Aug. 26, 2004, all of which are incorporated herein by reference. In certain embodiments, the compounds are an enantiomer of 2-[1-(3-ethoxy-4-methoxyphenyl)-2-methylsulfonylethyl]-4-acetylaminoisoindoline-1,3-dione and an enantiomer of 3-(3,4-dimethoxy-phenyl)-3-(1-oxo-1,3-dihydro-isoindol-2-yl)-propionamide.

In certain embodiments, the PDE4 modulators provided herein are 3-(3,4-dimethoxy-phenyl)-3-(1-oxo-1,3-dihydro-isoindol-2-yl)-propionamide and cyclopropanecarboxylic acid {2-[1-(3-ethoxy-4-methoxy-phenyl)-2-methanesulfonyl-ethyl]-3-oxo-2,3-dihydro-1H-isoindol-4-yl}-amide, which are available from Celgene Corp., Warren, N.J. 3-(3,4-Dimethoxy-phenyl)-3-(1-oxo-1,3-dihydro-isoindol-2-yl)-propionamide has the following chemical structure:

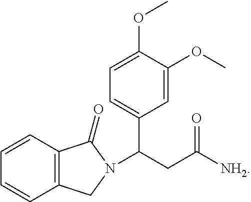 [SMILES: COc1ccc(C(CC(N)=O)N2Cc3ccccc3C2=O)cc1OC]

Other PDE4 modulators include, but are not limited to, the cycloalkyl amides and cycloalkyl nitriles of U.S. Pat. Nos. 5,728,844, 5,728,845, 5,968,945, 6,180,644 and 6,518,281, and WO 97/08143 and WO 97/23457, each of which is incorporated herein by reference. Representative compounds include those of formula:

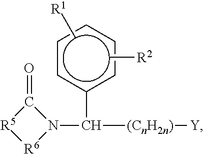

or a pharmaceutically acceptable salt, solvate, hydrate, clathrate, stereoisomer, or prodrug thereof, wherein:
one of R¹ and R² is R³—X— and the other is hydrogen, nitro, cyano, trifluoromethyl, carbo(lower)alkoxy, acetyl, carbamoyl, acetoxy, carboxy, hydroxy, amino, lower alkyl, lower alkoxy, halo, or R³—X—;
R³ is monocycloalkyl, bicycloalkyl, or benzocycloalkyl of up to 18 carbon atoms;
X is a carbon-carbon bond, —CH₂—, or —O—;
R⁵ is (i) o-phenylene, unsubstituted or substituted with 1 to 3 substituents each selected independently from nitro, cyano, halo, trifluoromethyl, carbo(lower)alkoxy, acetyl, or carbamoyl, unsubstituted or substituted with lower alkyl, acetoxy, carboxy, hydroxy, amino, lower alkylamino, lower acylamino, and lower alkoxy; (ii) a vicinally divalent residue of pyridine, pyrrolidine, imidazole, naphthalene, or thiophene, wherein the divalent bonds are on vicinal ring carbon atoms; (iii) a vicinally divalent cycloalkyl or cycloalkenyl of 4-10 carbon atoms, unsubstituted or substituted with 1 to 3 substituents each selected independently from the group consisting of nitro, cyano, halo, trifluoromethyl, carbo(lower)alkoxy, acetyl, carbamoyl, acetoxy, carboxy, hydroxy, amino, lower alkylamino, lower alkyl, lower alkoxy, or phenyl; (iv) vinylene di-substituted with lower alkyl; or (v) ethylene, unsubstituted or monosubstituted or disubstituted with lower alkyl;
R⁶ is —CO—, —CH₂—, or —CH₂CO—;
Y is —COZ, —C≡N, —OR⁸, lower alkyl, or aryl;
Z is —NH₂, —OH, —NHR, —R⁹, or —OR⁹
R⁸ is hydrogen or lower alkyl;
R⁹ is lower alkyl or benzyl; and
n has a value of 0, 1, 2, or 3.

In one embodiment, one of R¹ and R² is R³—X— and the other is hydrogen, nitro, cyano, trifluoromethyl, carbo(lower)alkoxy, acetyl, carbamoyl, acetoxy, carboxy, hydroxy, amino, lower alkyl, lower alkoxy, halo, or R³—X—;

R³ is monocycloalkyl of up to 10 carbon atoms, polycycloalkyl of up to 10 carbon atoms, or benzocyclic alkyl of up to 10 carbon atoms;

### X is —CH₂— or —O—;

R⁵ is (i) the vicinally divalent residue of pyridine, pyrrolidine, imidazole, naphthalene, or thiophene, wherein the two bonds of the divalent residue are on vicinal ring carbon atoms;
(ii) a vicinally divalent cycloalkyl of 4-10 carbon atoms, unsubstituted or substituted with 1 to 3 substituents each selected independently from the group consisting of nitro, cyano, halo, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, and phenyl;
(iii) di-substituted vinylene, substituted with nitro, cyano, trifluoromethyl, carbethoxy, carbomethoxy, carbopropoxy, acetyl, carbamoyl, carbamoyl substituted with an alkyl of 1 to 3 carbon atoms, acetoxy, carboxy, hydroxy, amino, amino substituted with an alkyl of 1 to 3 carbon atoms, alkyl of 1 to 4 carbon atoms, alkoxy of 1 to 4 carbon atoms, or halo;
(iv) ethylene, unsubstituted or substituted with 1 to 2 substituents each selected independently from nitro, cyano, trifluoromethyl, carbethoxy, carbomethoxy, carbopropoxy, acetyl, carbamoyl, carbamoyl substituted with an alkyl of 1 to 3 carbon atoms, acetoxy, carboxy, hydroxy, amino, amino substituted with an alkyl of 1 to 3 carbon atoms, alkyl of 1 to 4 carbon atoms, alkoxy of 1 to 4 carbon atoms, and halo;
R⁶ is —CO—, —CH₂—, or —CH₂CO—;
Y is —COX, —C≡N, —OR⁸, alkyl of 1 to 5 carbon atoms, or aryl;
X is —NH₂, —OH, —NHR, —R⁹, —OR⁹, or alkyl of 1 to 5 carbon atoms;
R⁸ is hydrogen or lower alkyl;
R⁹ is alkyl or benzyl; and,
n has a value of 0, 1, 2, or 3.

In another embodiment, one of R¹ and R² is R³—X— and the other is hydrogen, nitro, cyano, trifluoromethyl, carbo(lower)alkoxy, acetyl, carbamoyl, acetoxy, carboxy, hydroxy, amino, lower alkyl, lower alkoxy, halo, HF₂CO, F₃CO, or R³—X—;

R³ is monocycloalkyl, bicycloalkyl, benzocyclo alkyl of up to 18 carbon atoms, tetrahydropyran, or tetrahydrofuran;
X is a carbon-carbon bond, —CH₂—, —O—, or —N═;
R⁵ is (i) o-phenylene, unsubstituted or substituted with 1 to 3 substituents each selected independently from nitro, cyano, halo, trifluoromethyl, carbo(lower)alkoxy, acetyl, or carbamoyl, unsubstituted or substituted with lower alkyl, acetoxy, carboxy, hydroxy, amino, lower alkylamino, lower acylamino, and lower alkoxy; (ii) a vicinally divalent residue of pyridine, pyrrolidine, imidazole, naphthalene, or thiophene, wherein the divalent bonds are on vicinal ring carbon atoms; (iii) a vicinally divalent cycloalkyl or cycloalkenyl of 4-10 carbon atoms, unsubstituted or substituted with 1 or more substituents each selected independently from the group consisting of nitro, cyano, halo, trifluoromethyl, carbo(lower)alkoxy, acetyl, carbamoyl, acetoxy, carboxy, hydroxy, amino, lower alkylamino, lower alkyl, lower alkoxy, and phenyl; (iv) vinylene di-substituted with lower alkyl; or (v) ethylene, unsubstituted or monosubstituted or disubstituted with lower alkyl;
R⁶ is —CO—, —CH₂—, or —CH₂CO—;
Y is —COX, —C≡N, —OR⁸, alkyl of 1 to 5 carbon atoms, or aryl;
X is —NH₂, —OH, —NHR, —R⁹, —OR⁹, or alkyl of 1 to 5 carbon atoms;
R⁸ is hydrogen or lower alkyl;
R⁹ is alkyl or benzyl; and,
n has a value of 0, 1, 2, or 3.

Other representative compounds include those of formula:

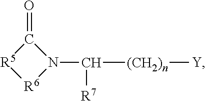

or a pharmaceutically acceptable salt, solvate, hydrate, clathrate, stereoisomer, or prodrug thereof, wherein:
Y is —C≡N or CO(CH₂)ₘCH₃;
m is 0, 1, 2, or 3;
R⁵ is (i) o-phenylene, unsubstituted or substituted with 1 to 3 substituents each selected independently from nitro, cyano, trifluoromethyl, carbethoxy, carbomethoxy, carbopropoxy, acetyl, carbamoyl, carbamoyl substituted with an alkyl of 1 to 3 carbon atoms, acetoxy, carboxy, hydroxy, amino, amino substituted with an alkyl of 1 to 3 carbon atoms, alkyl of 1 to 4 carbon atoms, alkoxy of 1 to 4 carbon atoms, and halo; (ii) the divalent residue of pyridine, pyrrolidine, imidizole, naphthalene, or thiophene, wherein the divalent bonds are on vicinal ring carbon atoms; (iii) a divalent cycloalkyl of 4-10 carbon atoms, unsubstituted or substituted with one or more substituents each selected independently of the other from the group consisting of nitro, cyano, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, phenyl and halo; (iv) di-substituted vinylene, substituted with nitro, cyano, trifluoromethyl, carbethoxy, carbomethoxy, carbopropoxy, acetyl, carbamoyl, carbamoyl substituted with an alkyl of 1 to 3 carbon atoms, acetoxy, carboxy, hydroxy, amino, amino substituted with an alkyl of 1 to 3 carbon atoms, alkyl of 1 to 4 carbon atoms, alkoxy of 1 to 4 carbon atoms, or halo; or (v) ethylene, unsubstituted or substituted with 1 to 2 substituents each selected independently from nitro, cyano, trifluoromethyl, carbethoxy, carbomethoxy, carbopropoxy, acetyl, carbamoyl, carbamoyl substituted with an alkyl of 1 to 3 carbon atoms, acetoxy, carboxy, hydroxy, amino, amino substituted with an alkyl of 1 to 3 carbon atoms, alkyl of 1 to 4 carbon atoms, alkoxy of 1 to 4 carbon atoms, and halo;
R⁶ is —CO—, —CH₂—, —CH₂CO—, or —SO₂—;
R⁷ is (i) straight or branched alkyl of 1 to 12 carbon atoms; (ii) cyclic or bicyclic alkyl of 1 to 12 carbon atoms; (iii) pyridyl; (iv) phenyl substituted with one or more substituents each selected independently of the other from nitro, cyano, trifluoromethyl, carbethoxy, carbomethoxy, carbopropoxy, acetyl, carbamoyl, acetoxy, carboxy, hydroxy, amino, straight, branched, cyclic, or bicyclic alkyl of 1 to 10 carbon atoms, straight, branched, cyclic, or bicyclic alkoxy of 1 to 10 carbon atoms, CH₂R where R is a cyclic or bicyclic alkyl of 1 to 10 carbon atoms, and halo; (v) benzyl substituted with one to three substituents each selected independently from the group consisting of nitro, cyano, trifluoromethyl, carbethoxy, carbomethoxy, carbopropoxy, acetyl, carbamoyl, acetoxy, carboxy, hydroxy, amino, alkyl of 1 to 4 carbon atoms, alkoxy of 1 to 10 carbon atoms, and halo; (vi) naphthyl; or (vii) benzyloxy; and
n has a value of 0, 1, 2, or 3.

In another embodiment, the PDE4 modulators include those of formula:

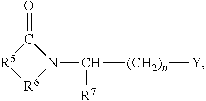

or a pharmaceutically acceptable salt, solvate, hydrate, clathrate, stereoisomer, or prodrug thereof, wherein:
R⁵ is (i) the divalent residue of pyridine, pyrrolidine, imidizole, naphthalene, or thiophene, wherein the divalent bonds are on vicinal ring carbon atoms; (ii) a divalent cycloalkyl of 4-10 carbon atoms, unsubstituted or substituted with one or more substituents each selected independently of the other from the group consisting of nitro, cyano, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, phenyl and halo; (iii) di-substituted vinylene, substituted with nitro, cyano, trifluoromethyl, carbethoxy, carbomethoxy, carbopropoxy, acetyl, carbamoyl, carbamoyl substituted with an alkyl of 1 to 3 carbon atoms, acetoxy, carboxy, hydroxy, amino, amino substituted with an alkyl of 1 to 3 carbon atoms, alkyl of 1 to 4 carbon atoms, alkoxy of 1 to 4 carbon atoms, or halo; or (iv) ethylene, unsubstituted or substituted with 1 to 2 substituents each selected independently from nitro, cyano, trifluoromethyl, carbethoxy, carbomethoxy, carbopropoxy, acetyl, carbamoyl, carbamoyl substituted with an alkyl of 1 to 3 carbon atoms, acetoxy, carboxy, hydroxy, amino, amino substituted with an alkyl of 1 to 3 carbon atoms, alkyl of 1 to 4 carbon atoms, alkoxy of 1 to 4 carbon atoms, or halo;
R⁶ is —CO—, —CH₂—, —CH₂CO—, or —SO₂—;
R⁷ is (i) cyclic or bicyclic alkyl of 4 to 12 carbon atoms; (ii) pyridyl; (iii) phenyl substituted with one or more substituents each selected independently of the other from nitro, cyano, trifluoromethyl, carbethoxy, carbomethoxy, carbopropoxy, acetyl, carbamoyl, acetoxy, carboxy, hydroxy, amino, straight, branched, cyclic, or bicyclic alkyl of 1 to 10 carbon atoms, straight, branched, cyclic, or bicyclic alkoxy of 1 to 10 carbon atoms, CH₂R where R is a cyclic or bicyclic alkyl of 1 to 10 carbon atoms, or halo; (iv) benzyl substituted with one to three substituents each selected independently from the group consisting of nitro, cyano, trifluoromethyl, carbethoxy, carbomethoxy, carbopropoxy, acetyl, carbamoyl, acetoxy, carboxy, hydroxy, amino, alkyl of 1 to 4 carbon atoms, alkoxy of 1 to 10 carbon atoms, and halo; (v) naphthyl; or (vi) benzyloxy; and
Y is COX, —C≡N, OR⁸, alkyl of 1 to 5 carbon atoms, or aryl;
X is —NH₂, —OH, —NHR, —R⁹, —OR⁹, or alkyl of 1 to 5 carbon atoms;
R⁸ is hydrogen or lower alkyl;
R⁹ is alkyl or benzyl; and
n has a value of 0, 1, 2, or 3.

Other PDE4 modulators include, but are not limited to, the aryl amides (for example, an embodiment being N-benzoyl-3-amino-3-(3′,4′-dimethoxyphenyl)-propanamide) of U.S. Pat. Nos. 5,801,195, 5,736,570, 6,046,221 and 6,284,780, each of which is incorporated herein by reference. Representative compounds include those of formula:

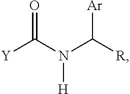

or a pharmaceutically acceptable salt, solvate, hydrate, clathrate, stereoisomer, or prodrug thereof, wherein:
Ar is (i) straight, branched, or cyclic, unsubstituted alkyl of 1 to 12 carbon atoms; (ii) straight, branched, or cyclic, substituted alkyl of 1 to 12 carbon atoms; (iii) phenyl; (iv) phenyl substituted with one or more substituents each selected independently of the other from the group consisting of nitro, cyano, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, and halo; (v) heterocycle; or (vi) heterocycle substituted with one or more substituents each selected independently of the other from nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo;
R is —H, alkyl of 1 to 10 carbon atoms, CH₂OH, CH₂CH₂OH, or CH₂COZ where Z is alkoxy of 1 to 10 carbon atoms, benzyloxy, or NHR¹ where R¹ is H or alkyl of 1 to 10 carbon atoms; and Y is i) a phenyl or heterocyclic ring, unsubstituted or substituted one or more substituents each selected independently of the other from nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo; or ii) naphthyl.

Other examples of the compounds include those of formula:

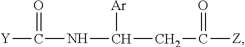

or a pharmaceutically acceptable salt, solvate, hydrate, clathrate, stereoisomer, or prodrug thereof, wherein:
Ar is 3,4-disubstituted phenyl where each substituent is selected independently of the other from the group consisting of nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo;
Z is alkoxy of 1 to 10 carbon atoms, benzyloxy, amino, or alkylamino of 1 to 10 carbon atoms; and
Y is (i) a phenyl, unsubstituted or substituted with one or more substituents each selected, independently one from the other, from the group consisting of nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo, or (ii) naphthyl.

Other PDE4 modulators include, but are not limited to, the imide/amide ethers and alcohols (for example, 3-phthalimido-3-(3′,4′-dimethoxyphenyl) propan-1-ol) disclosed in U.S. Pat. No. 5,703,098, which is incorporated herein by reference. Examples include compounds the formula:

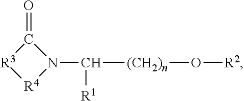

or a pharmaceutically acceptable salt, solvate, hydrate, clathrate, stereoisomer, or prodrug thereof, wherein:
R¹ is (i) straight, branched, or cyclic, unsubstituted alkyl of 1 to 12 carbon atoms; (ii) straight, branched, or cyclic, substituted alkyl of 1 to 12 carbon atoms; (iii) phenyl; or (iv) phenyl substituted with one or more substituents each selected independently of the other from the group consisting of nitro, cyano, trifluoromethyl, carbethoxy, carbomethoxy, carbopropoxy, acetyl, carbamoyl, acetoxy, carboxy, hydroxy, amino, acylamino, alkylamino, di(alkyl) amino, alkyl of 1 to 10 carbon atoms, cycloalkyl of 3 to 10 carbon atoms, bicycloalkyl of 5 to 12 carbon atoms, alkoxy of 1 to 10 carbon atoms, cycloalkoxy of 3 to 10 carbon atoms, bicycloalkoxy of 5 to 12 carbon atoms, and halo;
R² is hydrogen, alkyl of 1 to 8 carbon atoms, benzyl, pyridylmethyl, or alkoxymethyl;
R³ is (i) ethylene, (ii) vinylene, (iii) a branched alkylene of 3 to 10 carbon atoms, (iv) a branched alkenylene of 3 to 10 carbon atoms, (v) cycloalkylene of 4 to 9 carbon atoms unsubstituted or substituted with one or more substituents each selected independently from the group consisting of nitro, cyano, trifluoromethyl, carbethoxy, carbomethoxy, carbopropoxy, acetyl, carbamoyl, acetoxy, carboxy, hydroxy, amino, amino substituted with alkyl of 1 to 6 carbon atoms, amino substituted with acyl of 1 to 6 carbon atoms, alkyl of 1 to 10 carbon atoms, alkoxy of 1 to 12 carbon atoms, and halo, (vi) cycloalkenylene of 4 to 9 carbon atoms unsubstituted or substituted with one or more substituents each selected independently from the group consisting of nitro, cyano, trifluoromethyl, carbethoxy, carbomethoxy, carbopropoxy, acetyl, carbamoyl, acetoxy, carboxy, hydroxy, amino, amino substituted with alkyl of 1 to 6 carbon atoms, amino substituted with acyl of 1 to 6 carbon atoms, alkyl of 1 to 10 carbon atoms, alkoxy of 1 to 12 carbon atoms, and halo, (vii) o-phenylene unsubstituted or substituted with one or more substituents each selected independently from the group consisting of nitro, cyano, trifluoromethyl, carbethoxy, carbomethoxy, carbopropoxy, acetyl, carbamoyl, acetoxy, carboxy, hydroxy, amino, amino substituted with alkyl of 1 to 6 carbon atoms, amino substituted with acyl of 1 to 6 carbon atoms, alkyl of 1 to 10 carbon atoms, alkoxy of 1 to 12 carbon atoms, and halo, (viii) naphthyl, or (ix) pyridyl;
R⁴ is —CX—, —CH₂— or —CH₂CX—;

### X is O or S; and

n is 0, 1, 2, or 3.

Other PDE4 modulators include, but are not limited to, the succinimides and maleimides (for example methyl 3-(3′,4′,5′6′-petrahydrophthalimdo)-3-(3″,4″-dimethoxyphenyl)propionate) disclosed in U.S. Pat. No. 5,658,940, which is incorporated herein by reference. Examples include compounds of formula:

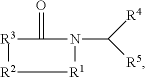

or a pharmaceutically acceptable salt, solvate, hydrate, clathrate, stereoisomer, or prodrug thereof, wherein:
R¹ is —CH₂—, —CH₂CO—, or —CO—;
R² and R³ taken together are (i) ethylene unsubstituted or substituted with alkyl of 1-10 carbon atoms or phenyl, (ii) vinylene substituted with two substituents each selected, independently of the other, from the group consisting of alkyl of 1-10 carbon atoms and phenyl, or (iii) a divalent cycloalkyl of 5-10 carbon atoms, unsubstituted or substituted with one or more substituents each selected independently of the other from the group consisting of nitro, cyano, trifluoromethyl, carbethoxy, carbomethoxy, carbopropoxy, acetyl, carbamoyl unsubstituted or substituted with alkyl of 1-3 carbon atoms, acetoxy, carboxy, hydroxy, amino, substituted amino, alkyl of 1 to 10 carbon atoms, alkoxy of 1 to 10 carbon atoms, norbornyl, phenyl and halo;
R⁴ is (i) straight or branched unsubstituted alkyl of 4 to 8 carbon atoms; (ii) cycloalkyl or bicycloalkyl of 5-10 carbon atoms, unsubstituted or substituted with one or more substituents each selected independently of the other from the group consisting of nitro, cyano, trifluoromethyl, carbethoxy, carbomethoxy, carbopropoxy, acetyl, carbamoyl, acetoxy, carboxy, hydroxy, amino, substituted amino, branched, straight or cyclic alkyl of 1 to 10 carbon atoms, alkoxy of 1 to 10 carbon atoms, phenyl and halo; (iii) phenyl substituted with one or more substituents each selected independently of the other from the group consisting of nitro, cyano, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, cycloalkyl or bicyctoalkyl of 3 to 10 carbon atoms, cycloalkoxy or bicycloalkoxy of 3 to 10 carbon atoms, phenyl and halo; (iv) pyridine or pyrrolidine, unsubstituted or substituted with one or more substituents each selected independently of the other from the group consisting of nitro, cyano, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, phenyl and halo;
R⁵ is —COX, —CN, —CH₂COX, alkyl of 1 to 5 carbon atoms, aryl, —CH₂OR, —CH₂ aryl, or —CH₂OH,
X is NH₂, OH, NHR, or OR⁶,
R is lower alkyl; and
R⁶ is alkyl or benzyl.

Other PDE4 modulators include, but are not limited to, substituted imides (for example, 2-phthalimido-3-(3′,4′-dimethoxyphenyl) propane) disclosed in U.S. Pat. No. 6,429,221, which is incorporated herein by reference. Examples include compounds of the formula:

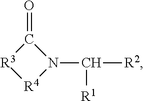

or a pharmaceutically acceptable salt, solvate, hydrate, clathrate, stereoisomer, or prodrug thereof, wherein:
R¹ is (i) straight, branched, or cyclic alkyl of 1 to 12 carbon atoms; (ii) phenyl or phenyl substituted with one or more substituents each selected independently of the other from nitro, cyano, trifluoromethyl, carbethoxy, carbomethoxy, carbopropoxy, acetyl, carbamoyl, acetoxy, carboxy, hydroxy, amino, straight or branched alkyl of 1 to 10 carbon atoms, alkoxy of 1 to 10 carbon atoms, and halo; (iii) benzyl or benzyl substituted with one or more substituents each selected independently of the other from nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo, or (iv) —Y-Ph where Y is a straight, branched, or cyclic alkyl of 1 to 12 carbon atoms and Ph is phenyl or phenyl substituted with one or more substituents each selected independently of the other from nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo;
R² is —H, a branched or unbranched alkyl of 1 to 10 carbon atoms, phenyl, pyridyl, heterocycle, —CH₂-aryl, or —CH₂-heterocycle;
R³ is i) ethylene; ii) vinylene; iii) a branched alkylene of 3 to 10 carbon atoms; iv) a branched alkenylene of 3 to 10 carbon atoms; v) cycloalkylene of 4 to 9 carbon atoms unsubstituted or substituted with 1 to 2 substituents each selected independently from nitro, cyano, trifluoromethyl, carbethoxy, carbomethoxy, carbopropoxy, acetyl, carbamoyl, acetoxy, carboxy, hydroxy, amino, substituted amino, alkyl of 1 to 4 carbon atoms, alkoxy of 1 to 4 carbon atoms, and halo; vi) cycloalkenylene of 4 to 9 carbon atoms unsubstituted or substituted with 1 to 2 substituents each selected independently from nitro, cyano, trifluoromethyl, carbethoxy, carbomethoxy, carbopropoxy, acetyl, carbamoyl, acetoxy, carboxy, hydroxy, amino, substituted amino, alkyl of 1 to 4 carbon atoms, alkoxy of 1 to 4 carbon atoms, or halo; or vii) o-phenylene unsubstituted or substituted with 1 to 2 substituents each selected independently from nitro, cyano, trifluoromethyl, carbethoxy, carbomethoxy, carbopropoxy, acetyl, carbamoyl, acetoxy, carboxy, hydroxy, amino, substituted amino, alkyl of 1 to 4 carbon atoms, alkoxy 1 to 4 carbon atoms, and halo;
R⁴ is —CX, or —CH₂—; and

### X is O or S.

Other PDE4 modulators include, but are not limited to, substituted 1,3,4-oxadiazoles (for example, 2-[1-(3-cyclopentyloxy-4-methoxyphenyl)-2-(1,3,4-oxadiazole-2-yl)ethyl]-5-methylisoindoline-1,3-dione) disclosed in U.S. Pat. No. 6,326,388, which is incorporated herein by reference. Examples include compounds of formula:

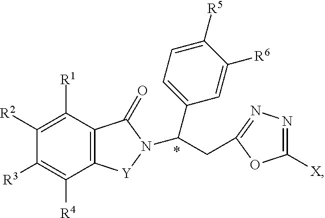

or a pharmaceutically acceptable salt, solvate, hydrate, clathrate, stereoisomer, or prodrug thereof, wherein:
the carbon atom designated * constitutes a center of chirality;
Y is C═O, CH₂, SO₂ or CH₂C═O;
X is hydrogen, or alkyl of 1 to 4 carbon atoms;
each of R¹, R², R³, and R⁴, independently of the others, is hydrogen, halo, trifluoromethyl, acetyl, alkyl of 1 to 8 carbon atoms, alkoxy of 1 to 4 carbon atoms, nitro, cyano, hydroxy, —CH₂NR⁸R⁹, —(CH₂)₂NR⁸R⁹, or —NR⁸R⁹ or
any two of R¹, R², R³, and R⁴ on adjacent carbon atoms, together with the depicted benzene ring are naphthylidene, quinoline, quinoxaline, benzimidazole, benzodioxole or 2-hydroxybenzimidazole;
each of R⁵ and R⁶, independently of the other, is hydrogen, alkyl of 1 to 4 carbon atoms, alkoxy of 1 to 6 carbon atoms, cyano, benzocycloalkoxy, cycloalkoxy of up to 18 carbon atoms, bicyloalkoxy of up to 18 carbon atoms, tricylcoalkoxy of up to 18 carbon atoms, or cycloalkylalkoxy of up to 18 carbon atoms;
each of R⁸ and R⁹, taken independently of the other is hydrogen, straight or branched alkyl of 1 to 8 carbon atoms, phenyl, benzyl, pyridyl, or pyridylmethyl; or one of R⁸ and R⁹ is hydrogen and the other is —COR¹⁰, or —SO₂R¹⁰; or R⁸ and R⁹ taken together are tetramethylene, pentamethylene, hexamethylene, —CH═NCH═CH—, or —CH₂CH₂X¹CH₂CH₂— in which X¹ is —O—, —S—, or —NH—;
R¹⁰ is hydrogen, alkyl of 1 to 8 carbon atoms, cycloalkyl, cycloalkylmethyl of up to 6 carbon atoms, phenyl, pyridyl, benzyl, imidazolylmethyl, pyridylmethyl, NR¹¹R¹², CH₂R¹⁴R¹⁵, or NR¹¹R¹²;
R¹⁴ and R¹⁵, independently of each other, are hydrogen, methyl, ethyl, or propyl; and
R¹¹ and R¹², independently of each other, are hydrogen, alkyl of 1 to 8 carbon atoms, phenyl, or benzyl.

In certain embodiments, the compounds include those of formula:

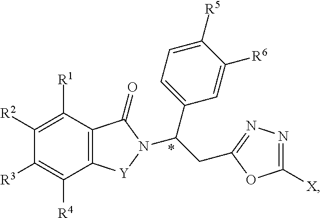

or a pharmaceutically acceptable salt, solvate, hydrate, clathrate, stereoisomer, or prodrug thereof, wherein:
the carbon atom designated * constitutes a center of chirality;
Y is C═O, CH₂, SO₂ or CH₂C═O;
X is hydrogen, or alkyl of 1 to 4 carbon atoms;
(i) each of R¹, R², R³, and R⁴, independently of the others, is hydrogen, halo, trifluoromethyl, acetyl, alkyl of 1 to 8 carbon atoms, alkoxy of 1 to 4 carbon atoms, nitro, cyano, hydroxy, —CH₂NR⁸R⁹, —(CH₂)₂NR⁸R⁹, or —NR⁸R⁹; or
(ii) any two of R¹, R², R³, and R⁴ on adjacent carbon atoms, together with the depicted benzene ring to which they are bound are naphthylidene, quinoline, quinoxaline, benzimidazole, benzodioxole or 2-hydroxybenzimidazole;
each of R⁵ and R⁶, independently of the other, is hydrogen, alkyl of 1 to 4 carbon atoms, alkoxy of 1 to 6 carbon atoms, cyano, benzocycloalkoxy, cycloalkoxy of up to 18 carbon atoms, bicyloalkoxy of up to 18 carbon atoms, tricylcoalkoxy of up to 18 carbon atoms, or cycloalkylalkoxy of up to 18 carbon atoms;
(i) each of R⁸ and R⁹, independently of the other, is hydrogen, alkyl of 1 to 8 carbon atoms, phenyl, benzyl, pyridyl, or pyridylmethyl; or
(ii) one of R⁸ and R⁹ is hydrogen and the other is —COR¹⁰ or —SO₂R¹⁰, in which R¹⁰ is hydrogen, alkyl of 1 to 8 carbon atoms, cycloalkyl, cycloalkylmethyl of up to 6 carbon atoms, phenyl, pyridyl, benzyl, imidazolylmethyl, pyridylmethyl, or NR¹¹R¹², or CH₂NR¹⁴R¹⁵, wherein R¹¹ and R¹², independently of each other, are hydrogen, alkyl of 1 to 8 carbon atoms, phenyl, or benzyl and R¹⁴ and R¹⁵, independently of each other, are hydrogen, methyl, ethyl, or propyl; or
(iii) R⁸ and R⁹ taken together are tetramethylene, pentamethylene, hexamethylene, —CH═NCH═CH—, or —CH₂CH₂X¹CH₂CH₂—, in which X¹ is —O—, —S—, or —NH—.

Other PDE4 modulators include, but are not limited to, cyano and carboxy derivatives of substituted styrenes (for example, 3,3-bis-(3,4-dimethoxyphenyl) acrylonitrile) disclosed in U.S. Pat. Nos. 5,929,117, 6,130,226, 6,262,101 and 6,479,554, each of which is incorporated herein by reference. Examples include compounds of formula:

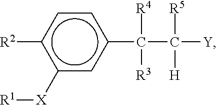

or a pharmaceutically acceptable salt, solvate, hydrate, clathrate, stereoisomer, or prodrug thereof, wherein:
(a) X is —O— or —(CₙH₂ₙ)— in which n has a value of 0, 1, 2, or 3, and R¹ is alkyl of one to 10 carbon atoms, monocycloalkyl of up to 10 carbon atoms, polycycloalkyl of up to 10 carbon atoms, or benzocyclic alkyl of up to 10 carbon atoms; or
(b) X is —CH═ and R¹ is alkylidene of up to 10 carbon atoms, monocycloalkylidene of up to 10 carbon atoms, or bicycloalkylidene of up to 10 carbon atoms;
R² is hydrogen, nitro, cyano, trifluoromethyl, carbethoxy, carbomethoxy, carbopropoxy, acetyl, carbamoyl, acetoxy, carboxy, hydroxy, amino, lower alkyl, lower alkylidenemethyl, lower alkoxy, or halo;
R³ is (i) phenyl, unsubstituted or substituted with 1 or more substituents each selected independently from nitro, cyano, halo, trifluoromethyl, carbethoxy, carbomethoxy, carbopropoxy, acetyl, carbamoyl, carbamoyl substituted with alkyl of 1 to 3 carbon atoms, acetoxy, carboxy, hydroxy, amino, amino substituted with an alkyl of 1 to 5 carbon atoms, alkyl of up to 10 carbon atoms, cycloalkyl of up to 10 carbon atoms, alkoxy of up to 10 carbon atoms, cycloalkoxy of up to 10 carbon atoms, alkylidenemethyl of up to 10 carbon atoms, cycloalkylidenemethyl of up to 10 carbon atoms, phenyl, and methylenedioxy; (ii) pyridine, substituted pyridine, pyrrolidine, imidizole, naphthalene, or thiophene; (iii) cycloalkyl of 4-10 carbon atoms, unsubstituted or substituted with 1 or more substituents each selected independently from the group consisting of nitro, cyano, halo, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, and alkoxy of 1 to 10 carbon atoms, phenyl; each of R⁴ and R⁵ taken individually is hydrogen; or R⁴ and R⁵ taken together are a carbon-carbon bond;
Y is —COZ, —C≡N, or lower alkyl of 1 to 5 carbon atoms;
Z is —OH, —NR⁶R⁶, —R⁷, or —OR⁷; R⁶ is hydrogen or lower alkyl; and R⁷ is alkyl or benzyl.

In some embodiments, PDE4 modulators include compounds of formula:

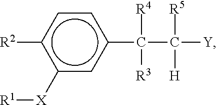

or a pharmaceutically acceptable salt, solvate, hydrate, clathrate, stereoisomer, or prodrug thereof, wherein:
(a) X is —O— or —(CₙH₂ₙ)— in which n has a value of 0, 1, 2, or 3, and R¹ is alkyl of one to 10 carbon atoms, monocycloalkyl of up to 10 carbon atoms, polycycloalkyl of up to 10 carbon atoms, or benzocyclic alkyl of up to 10 carbon atoms; or
(b) X is —CH═ and R¹ is alkylidene of up to 10 carbon atoms, monocycloalkylidene of up to 10 carbon atoms, or bicycloalkylidene of up to 10 carbon atoms;
R² is hydrogen, nitro, cyano, trifluoromethyl, carbethoxy, carbomethoxy, carbopropoxy, acetyl, carbamoyl, acetoxy, carboxy, hydroxy, amino, lower alkyl, lower alkylidenemethyl, lower alkoxy, or halo;
R³ is pyrrolidine, imidazole or thiophene unsubstituted or substituted with 1 or more substituents each selected independently from the group consisting of nitro, cyano, halo, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, and phenyl; each of R⁴ and R⁵ taken individually is hydrogen; or R⁴ and R⁵ taken together are a carbon-carbon bond;
Y is —COZ, —C≡N, or lower alkyl of 1 to 5 carbon atoms;
Z is —OH, —NR⁶R⁶, —R⁷, or —OR⁷; R⁶ is hydrogen or lower alkyl; and R⁷ is alkyl or benzyl.

In some embodiments, provided herein are compounds of the formula:

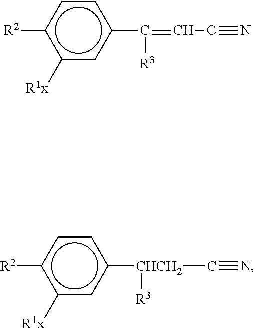

or a pharmaceutically acceptable salt, solvate, hydrate, clathrate, stereoisomer, or prodrug thereof, wherein:
(a) X is —O— or —(CₙH₂ₙ)— in which n has a value of 0, 1, 2, or 3, and R¹ is alkyl of up to 10 carbon atoms, monocycloalkyl of up to 10 carbon atoms, polycycloalkyl of up to 10 carbon atoms, or benzocyclic alkyl of up to 10 carbon atoms, or
(b) X is —CH═, and R¹ is alkylidene of up to 10 carbon atoms or monocycloalkylidene of up to 10 carbon atoms;
R² is hydrogen, nitro, cyano, trifluoromethyl, carbethoxy, carbomethoxy, carbopropoxy, acetyl, carbamoyl, acetoxy, carboxy, hydroxy, amino, lower alkyl, lower alkoxy, or halo; and
R³ is (i) phenyl or naphthyl, unsubstituted or substituted with 1 or more substituents each selected independently from nitro, cyano, halo, trifluoromethyl, carbethoxy, carbomethoxy, carbopropoxy, acetyl, carbamoyl, or carbamoyl substituted with alkyl of 1 to 3 carbon atoms, acetoxy, carboxy, hydroxy, amino, amino substituted with an alkyl of 1 to 5 carbon atoms, alkoxy and cycloalkoxy of 1 to 10 carbon atoms; or (ii) cycloalkyl of 4 to 10 carbon atoms, unsubstituted or substituted with one or more substituents each selected independently from the group consisting of nitro, cyano, halo, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, and phenyl.

In one embodiment, the compound is of formula:

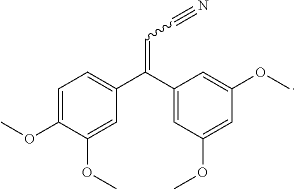

Other PDE4 modulators include, but are not limited to, isoindoline-1-one and isoindoline-1,3-dione substituted in the 2-position with an α-(3,4-disubstituted phenyl)alkyl group and in the 4- and/or 5-position with a nitrogen-containing group disclosed in WO 01/34606 and U.S. Pat. No. 6,667,316, which are incorporated herein by reference. Examples include compounds of formula:

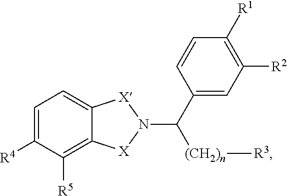

or a pharmaceutically acceptable salt, solvate, hydrate, clathrate, stereoisomer, or prodrug thereof, wherein:
one of X and X′ is ═C═O or ═SO₂, and the other of X and X′ is ═C═O, ═CH₂, ═SO₂ or ═CH₂C═O;
n is 1, 2 or 3;
R¹ and R² are each independently (C₁-C₄)alkyl, (C₁-C₄)alkoxy, cyano, (C₃-C₁₈)cycloalkyl, (C₃-C₁₈)cycloalkoxy, or (C₃-C₁₈)cycloalkyl-methoxy;
R³ is SO₂—Y, COZ, CN or (C₁-C₆)hydroxyalkyl, wherein:
Y is (C₁-C₆)alkyl, benzyl or phenyl;
Z is —NR⁶R⁷, (C₁-C₆)alkyl, benzyl or phenyl;
R⁶ is H, (C₁-C₄)alkyl, (C₃-C₁₈)cycloalkyl, (C₂-C₅)alkanoyl, benzyl or phenyl, each of which may be optionally substituted with halo, amino or (C₁-C₄)alkyl-amino;
R⁷ is H or (C₁-C₄)alkyl;
R⁴ and R⁵ are taken together to provide —NH—CH₂—R⁸—, NH—CO—R⁸—, or —N═CH—R⁸—, wherein:
R⁸ is CH₂, O, NH, CH═CH, CH═N, or N═CH; or
one of R⁴ and R⁵ is H, and the other of R₄ and R₅ is imidazoyl, pyrrolyl, oxadiazolyl, triazolyl, or a structure of formula (A),

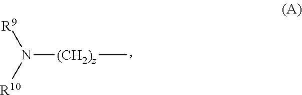

wherein:
z is 0 or 1;
R⁹ is: H; (C₁-C₄)alkyl, (C₃-C₁₈)cycloalkyl, (C₂-C₅)alkanoyl, or (C₄-C₆)cycloalkanoyl, optionally substituted with halo, amino, (C₁-C₄)alkyl-amino, or (C₁-C₄)dialkyl-amino; phenyl; benzyl; benzoyl; (C₂-C₅)alkoxycarbonyl; (C₃-C₅)alkoxyalkylcarbonyl; N-morpholinocarbonyl; carbamoyl; N-substituted carbamoyl substituted with (C₁-C₄)alkyl; or methylsulfonyl; and
R¹⁰ is H, (C₁-C₄)alkyl, methylsulfonyl, or (C₃-C₅)alkoxyalkylcarbonyl; or
R⁹ and R¹⁰ are taken together to provide —CH═CH—CH═CH—, —CH═CH—N═CH—, or (C₁-C₂)alkylidene, optionally substituted with amino, (C₁-C₄)alkyl-amino, or (C₁-C₄)dialkyl-amino; or
R⁴ and R⁵ are both structures of formula (A).

In one embodiment, z is not 0 when (i) R³ is —SO₂—Y, —COZ, or —CN and (ii) one of R⁴ or R⁵ is hydrogen. In another embodiment, R⁹ and R¹⁰, taken together, are —CH═CH—CH═CH—, —CH═CH—N═CH—, or (C₁-C₂)alkylidene substituted by amino, (C₁-C₄)alkyl-amino, or (C₁-C₄)dialkyl-amino. In another embodiment, R⁴ and R⁵ are both structures of formula (A).

In some embodiments, compounds include those of formula:

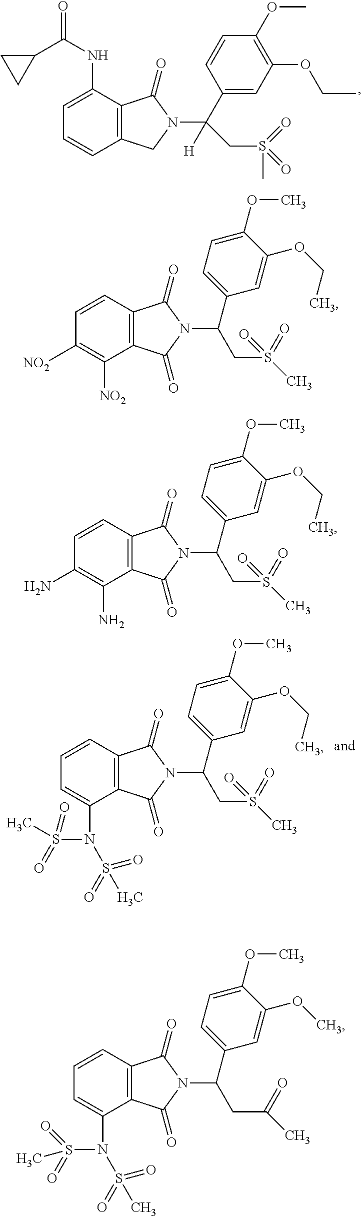

or a pharmaceutically acceptable salt, solvate, hydrate, clathrate, stereoisomer, or prodrug thereof.

Further examples include, but are not limited to: 2-[1-(3-Ethoxy-4-methoxyphenyl)-2-methylsulfonylethyl]-4,5-dinitroisoindoline-1,3-dione; 2-[1-(3-Ethoxy-4-methoxyphenyl)-2-methylsulfonylethyl]-4,5-diaminoisoindoline-1,3-dione; 7-[1-(3-Ethoxy-4-methoxyphenyl)-2-methylsulfonylethyl]-3-pyrrolino[3,4-e]benzimidazole-6,8-dione; 7-[1-(3-Ethoxy-4-methoxyphenyl)-2-methylsulfonylethyl]hydro-3-pyrrolino[3,4-e]benzimidazole-2,6,8-trione; 2-[1-(3-Ethoxy-4-methoxyphenyl)-2-methylsulfonylethyl]-3-pyrrolino[3,4-f]quinoxaline-1,3-dione; Cyclopropyl-N-{2-[1-(3-ethoxy-4-methoxyphenyl)-2-methylsulfonylethyl]-1,3-dioxoisoindolin-4-yl}carboxamide; 2-Chloro-N-{2-[1-(3-ethoxy-4-methoxyphenyl)-2-methylsulfonylethyl]-1,3-dioxoisoindolin-4-yl}acetamide; 2-Amino-N-{2-[1-(3-ethoxy-4-methoxyphenyl)-2-methylsulfonylethyl]-1,3-dioxoisoindolin-4-yl}acetamide; 2-N,N-Dimethylamino-N-{2-[-(3-ethoxy-4-methoxyphenyl)-2-methylsulfonylethyl]-1,3-dioxoisoindolin-4-yl}acetamide; N-{2-[1-(3-ethoxy-4-methoxyphenyl)-2-methylsulfonylethyl]-1,3-dioxoisoindolin-4-yl}-2,2,2-trifluoroacetamide; N-{2-[1-(3-Ethoxy-4-methoxyphenyl)-2-methylsulfonylethyl]-1,3-dioxoisoindolin-4-yl}methoxycarboxamide; 4-[1-Aza-2-(dimethylamino)vinyl]-2-[1-(3-ethoxy-4-methoxyphenyl)-2-methylsulfonylethyl]isoindoline-1,3-dione; 4-[1-Aza-2-(dimethylamino)prop-1-enyl]-2-[1-(3-ethoxy-4-methoxyphenyl)-2-methylsulfonylethyl]isoindoline-1,3-dione; 2-[1-(3-Ethoxy-4-methoxyphenyl)-2-methylsulfonylethyl]-4-(5-methyl-1,3,4-oxadiazol-2-yl)isoindoline-1,3-dione; 2-[1-(3-Ethoxy-4-methoxyphenyl)-2-methylsulfonylethyl]-4-pyrrolylisoindoline-1,3-dione; 4-(Aminomethyl)-2-[1-(3-ethoxy-4-methoxyphenyl)-2-methylsulfonylethyl]-isoindoline-1,3-dione; 2-[1-(3-Ethoxy-4-methoxyphenyl)-2-methylsulfonylethyl]-4-(pyrrolylmethyl)isoindoline-1,3-dione; N-{2-[1-(3-ethoxy-4-methoxyphenyl)-3-hydroxybutyl]-1,3-dioxoisoindolin-4-yl}acetamide; N-{2-[1-(3-Ethoxy-4-methoxyphenyl)-3-oxobutyl]-1,3-dioxoisoindolin-4-yl}acetamide; N-{2-[1R-(3-ethoxy-4-methoxyphenyl)-3-hydroxybutyl]-1,3-dioxoisoindolin-4-yl}acetamide; N-{2-[1R-(3-ethoxy-4-methoxyphenyl)-3-oxobutyl]-1,3-dioxoisoindolin-4-yl}acetamide; N-{2-[1S-(3-Ethoxy-4-methoxyphenyl)-3-hydroxybutyl]-1,3-dioxoisoindolin-4-yl}acetamide; N-{2-[1S-(3-ethoxy-4-methoxyphenyl)-3-oxobutyl]-1,3-dioxoisoindolin-4-yl}acetamide; 4-Amino-2-[1-(3-ethoxy-4-methoxyphenyl)-3-hydroxybutylisoindoline-1,3-dione; 4-Amino-2-[1-(3-ethoxy-4-methoxyphenyl)-3-oxobutyl]isoindoline-1,3-dione; 2-[1-(3-Ethoxy-4-methoxyphenyl)-3-oxobutyl]-4-pyrrolylisoindoline-1,3-dione; 2-Chloro-N-{2-[1-(3-ethoxy-4-methoxyphenyl)-3-oxobutyl]-1,3-dioxoisoindol-4-yl}acetamide; 2-(Dimethylamino)-N-{2-[1-(3-ethoxy-4-methoxyphenyl)-3-oxobutyl]-1,3-dioxoisoindolin-4-yl}acetamide; 4-Amino-2-[1R-(3-ethoxy-4-methoxyphenyl)-3-hydroxybutyl]isoindoline-1,3-dione; 4-Amino-2-[1R-(3-ethoxy-4-methoxyphenyl)-3-oxobutyl]isoindoline-1,3-dione; 2-[1R-(3-ethoxy-4-methoxyphenyl)-3-oxobutyl]-4-pyrrolylisoindoline-1,3-dione; 2-(Dimethylamino)-N-{2-[1R-(3-ethoxy-4-methoxyphenyl)-3-oxobutyl]-1,3-dioxoisoindolin-4-yl}acetamide; Cyclopentyl-N-{2-[1-(3-ethoxy-4-methoxyphenyl)-2-(methylsulfonyl)ethyl]-1,3-dioxoisoindolin-4-yl}carboxamide; 3-(Dimethylamino)-N-{2-[1-(3-ethoxy-4-methoxyphenyl)-2-(methylsulfonyl)ethyl]-1,3-dioxoisoindolin-4-yl}propanamide; 2-(Dimethylamino)-N-{2-[1-(3-ethoxy-4-methoxyphenyl)-2-(methylsulfonyl)ethyl]-1,3-dioxoisoindolin-4-yl}propanamide; N-{2-[(1R)-1-(3-ethoxy-4-methoxyphenyl)-2-(methylsulfonyl)ethyl]-1,3-dioxoisoindolin-4-yl}-2-(dimethylamino)acetamide; N-{2-[(1S)-1-(3-ethoxy-4-methoxyphenyl)-2-(methylsulfonyl)ethyl]-1,3-dioxoisoindolin-4-yl}-2-(dimethylamino)acetamide; 4-{3-[(Dimethylamino)methyl]pyrrolyl}-2-[1-(3-ethoxy-4-methoxyphenyl)-2-(methylsulfonyl)ethyl]isoindoline-1,3-dione; Cyclopropyl-N-{2-[(1S)-1-(3-ethoxy-4-methoxyphenyl)-2-(methylsulfonyl)ethyl]-1,3-dioxoisoindolin-4-yl}carboxamide; 2-[1-(3,4-dimethoxyphenyl)-2-(methylsulfonyl)ethyl]-4-pyrrolylisoindoline-1,3-dione; N-{2-[1-(3,4-dimethoxyphenyl)-2-(methylsulfonyl)ethyl]-1,3-dioxoisoindolin-4-yl}-2-(dimethylamino)acetamide; Cyclopropyl-N-{2-[1-(3,4-dimethoxyphenyl)-2-(methylsulfonyl)ethyl]-1,3-dioxoisoindolin-4-yl}carboxamide; Cyclopropyl-N-{2-[1-(3-ethoxy-4-methoxyphenyl)-2-(methylsulfonyl)ethyl]-3-oxoisoindolin-4-yl}carboxamide; 2-(Dimethylamino)-N-{2-[1-(3-ethoxy-4-methoxyphenyl)-2-(methylsulfonyl)ethyl]-3-oxoisoindolin-4-yl}acetamide; Cyclopropyl-N-{2-[(1S)-1-(3-ethoxy-4-methoxyphenyl)-2-(methylsulfonyl)ethyl]-3-oxoisoindolin-4-yl}carboxamide; Cyclopropyl-N-{2-[(1R)-1-(3-ethoxy-4-methoxyphenyl)-2-(methylsulfonyl)ethyl]-3-oxoisoindolin-4-yl}carboxamide; (3R)-3-[7-(Acetylamino)-1-oxoisoindolin-2-yl]-3-(3-ethoxy-4-methoxyphenyl)-N,N-dimethylpropanamide; (3R)-3-[7-(Cyclopropylcarbonylamino)-1-oxoisoindolin-2-yl]-3-(3-ethoxy-4-methoxyphenyl)-N,N-dimethylpropanamide; 3-{4-[2-(Dimethylamino)acetylamino]-1,3-dioxoisoindolin-2-yl}-3-(3-ethoxy-4-methoxyphenyl)-N,N-dimethylpropanamide; (3R)-3-[7-(2-Chloroacetylamino)-1-oxoisoindolin-2-yl]-3-(3-ethoxy-4-methoxy-phenyl)-N,N-dimethylpropanamide; (3R)-3-{4-[2-(dimethylamino)acetylamino]-1,3-dioxoisoindolin-2-yl}-3-(3-ethoxy-4-methoxyphenyl)-N,N-dimethylpropanamide; 3-(1,3-Dioxo-4-pyrrolylisoindolin-2-yl)-3-(3-ethoxy-4-methoxyphenyl)-N,N-dimethylpropanamide; 2-[1-(3-Ethoxy-4-methoxyphenyl)-2-(methylsulfonyl)ethyl]-4-(imidazolyl-methyl)isoindoline-1,3-dione; N-({2-[1-(3-Ethoxy-4-methoxyphenyl)-2-(methylsulfonyl)ethyl]-1,3-dioxoisoindolin-4-yl}methyl)acetamide; 2-Chloro-N-({2-[1-(3-ethoxy-4-methoxyphenyl)-2-(methylsulfonyl)ethyl]-1,3-dioxoisoindolin-4-yl}methyl)acetamide; 2-(Dimethylamino)-N-({2-[1-(3-ethoxy-4-methoxyphenyl)-2-(methylsulfonyl)ethyl]-1,3-dioxoisoindolin-4-yl}methyl)acetamide; 4-[Bis(methylsulfonyl)amino]-2-[1-(3-ethoxy-4-methoxyphenyl)-2-(methylsulfonyl)ethyl]isoindoline-1,3-dione; 2-[1-(3-Ethoxy-4-methoxyphenyl)-2-(methylsulfonyl)ethyl]-4-[(methylsulfonyl)amino]isoindoline-1,3-dione; N-{2-[1-(3-Ethoxy-4-methoxyphenyl)-3-hydroxypentyl]-1,3-dioxoisoindolin-4-yl}acetamide; N-{2-[1-(3-Ethoxy-4-methoxyphenyl)-3-oxopentyl]1,3-dioxoisoindolin-4-yl}acetamide; 2-[(1R)-1-(3-Ethoxy-4-methoxyphenyl)-3-hydroxybutyl]-4-(pyrrolylmethyl)isoindoline-1,3-dione; 2-[(1R)-1-(3-Ethoxy-4-methoxyphenyl)-3-oxobutyl]-4-(pyrrolylmethyl)isoindoline-1,3-dione; N-{2-[1-(3-Cyclopentyloxy-4-methoxyphenyl)-3-hydroxybutyl]-1,3-dioxoisoindolin-4-yl}acetamide; N-{2-[1-(3-Cyclopentyloxy-4-methoxyphenyl)-3-oxobutyl]-1,3-dioxoisoindolin-4-yl}acetamide; 2-[1-(3-Cyclopentyloxy-4-methoxyphenyl)-3-oxobutyl]-4-pyrrolylisoindoline-1,3-dione; 2-[1-(3,4-Dimethoxyphenyl)-3-oxobutyl]-4-[bis(methylsulfonyl)amino]isoindoline-1,3-dione; or a pharmaceutically acceptable salt, solvate, hydrate, clathrate, stereoisomer, or prodrug thereof.

Still other PDE4 modulators include, but are not limited to, imido and amido substituted acylhydroxamic acids (for example, (3-(1,3-dioxoisoindoline-2-yl)-3-(3-ethoxy-4-methoxyphenyl) propanoylamino) propanoate disclosed in WO 01/45702 and U.S. Pat. No. 6,699,899, which are incorporated herein by reference. Examples include compounds of formula:

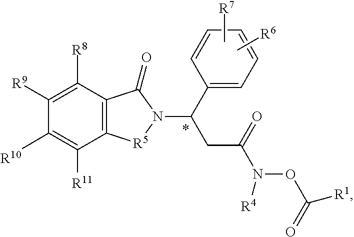

or a pharmaceutically acceptable salt, solvate, hydrate, clathrate, stereoisomer, or prodrug thereof, wherein:
the carbon atom designated * constitutes a center of chirality,
R⁴ is hydrogen or —(C═O)—R¹²,
each of R¹ and R¹², independently of each other, is alkyl of 1 to 6 carbon atoms, phenyl, benzyl, pyridyl methyl, pyridyl, imidazoyl, imidazolyl methyl, or CHR*(CH₂)ₙNR*R⁰,
wherein R*and R⁰, independently of the other, are hydrogen, alkyl of 1 to 6 carbon atoms, phenyl, benzyl, pyridyl methyl, pyridyl, imidazoyl or imidazolylmethyl, and n=0, 1, or 2;
R⁵ is C═O, CH₂, CH₂—CO—, or SO₂;
each of R⁶ and R⁷, independently of the other, is nitro, cyano, trifluoromethyl, carbethoxy, carbomethoxy, carbopropoxy, acetyl, carbamoyl, acetoxy, carboxy, hydroxy, amino, alkyl of 1 to 6 carbon atoms, alkoxy of 1 to 6 carbon atoms, cycloalkoxy of 3 to 8 carbon atoms, halo, bicycloalkyl of up to 18 carbon atoms, tricycloalkoxy of up to 18 carbon atoms, 1-indanyloxy, 2-indanyloxy, C₄-C₈-cycloalkylidenemethyl, or C₃-C₁₀-alkylidenemethyl;
each of R⁸, R⁹, R¹⁰, and R¹¹, independently of the others, is
(i) hydrogen, nitro, cyano, trifluoromethyl, carbethoxy, carbomethoxy, carbopropoxy, acetyl, carbamoyl, acetoxy, carboxy, hydroxy, amino, alkylamino, dialkylamino, acylamino, alkyl of 1 to 10 carbon atoms, or alkoxy of 1 to 10 carbon atoms, halo; or
(ii) one of R⁸, R⁹, R¹⁰, and R¹¹ is acylamino comprising a lower alkyl, and the remaining of R⁸, R⁹, R¹⁰, and R¹¹ are hydrogen; or
(iii) hydrogen if R⁸ and R⁹ taken together are benzo, quinoline, quinoxaline, benzimidazole, benzodioxole, 2-hydroxybenzimidazole, methylenedioxy, dialkoxy, or dialkyl; or
(iv) hydrogen if R¹⁰ and R¹¹, taken together are benzo, quinoline, quinoxaline, benzimidazole, benzodioxole, 2-hydroxybenzimidazole, methylenedioxy, dialkoxy, or dialkyl; or
(v) hydrogen if R⁹ and R¹⁰ taken together are benzo.

Still other PDE4 modulators include, but are not limited to, 7-amido-isoindolyl compounds disclosed in U.S. Pat. No. 7,034,052, which is incorporated herein by reference. Examples include compounds of formula:

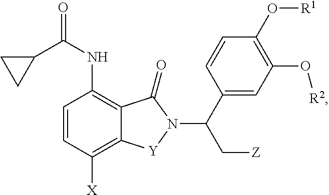

or a pharmaceutically acceptable salt, solvate, hydrate, clathrate, stereoisomer, or prodrug thereof, wherein:
Y is —C(O)—, —CH₂, —CH₂C(O)— or SO₂;

### X is H;

Z is (C₀₋₄-alkyl)-C(O)R³, C₁₋₄-alkyl, (C₀₋₄ alkyl)-OH, (C₁₋₄-alkyl)-O(C₁₋₄-alkyl), (C₁₋₄-alkyl)-SO₂(C₁₋₄-alkyl), (C₀₋₄-alkyl)-SO(C₁₋₄-alkyl), (C₀₋₄-alkyl)-NH₂, (C₀₋₄-alkyl)-N(C₁₋₈akyl)₂, (C₀₋₄-alkyl)-N(H)(OH), or CH₂NSO₂(C₁₋₄-alkyl);
R₁ and R₂ are independently C₁₋₈-alkyl, cycloalkyl, or (C₁₋₄-alkyl)cycloalkyl;
R³ is NR⁴R⁵, OH, or O—(C₁₋₈-alkyl);

### R⁴ is H;

R⁵ is —OH or —OC(O)R⁶; and
R⁶ is C₁₋₈-alkyl, amino-(C₁₋₈-alkyl), (C₁₋₈-alkyl)-(C₃₋₆-cycloalkyl), C₃₋₆-cycloalkyl, phenyl, benzyl, or aryl.

In other embodiments, provided herein is a compound of the following formula:

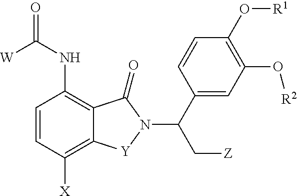

or a pharmaceutically acceptable salt, solvate, hydrate, clathrate, stereoisomer, or prodrug thereof, wherein:
Y is —C(O)—, —CH₂, —CH₂C(O)—, or SO₂;
X is halogen, —CN, —NR⁸R⁹, —NO₂, or —CF₃;
Z is (C₀₋₄alkyl)-SO₂(C₁₋₄-alkyl), —(C₀₋₄-alkyl)-CN, —(C₀₋₄-alkyl)-C(O)R³, C₁₋₄-alkyl, (C₀₋₄ alkyl)OH, (C₀₋₄-alkyl)O(C₁₋₄-alkyl), (C₀₋₄-alkyl)SO(C₁₋₄-alkyl), (C₀₋₄-alkyl)NH₂, (C₀₋₄-alkyl)N(C₁₋₈-alkyl)₂, (C₀₋₄-alkyl) N(H)(OH), (C₀₋₄-alkyl)-dichloropyridine or (C₀₋₄-alkyl)NSO₂(C₁₋₄-alkyl);
W is —C₃₋₆-cycloalkyl, —(C₁₋₈-alkyl)-(C₃₋₆-cycloalkyl), —(C₀₋₈-alkyl)-(C₃₋₆-cycloalkyl)NR₇R₈, (C₀₋₈-alkyl)-NR₇R₈, (C₀₋₄alkyl)-CHR₉—(C₀₋₄alkyl)-NR₇R₈;
R¹ and R² are independently C₁₋₈-alkyl, cycloalkyl, or (C₁₋₄-alkyl)cycloalkyl;
R³ is C₁₋₈-alkyl, NR⁴R⁵, OH, or O—(C₁₋₈-alkyl);
R⁴ and R⁵ are independently H, C₁₋₈-alkyl, (C₀₋₈-alkyl)-(C₃₋₆-cycloalkyl), OH, or —OC(O)R⁶;
R⁶ is C₁₋₈-alkyl, (C₀₋₈-alkyl)-(C₃₋₆-cycloalkyl), amino-(C₁₋₈-alkyl), phenyl, benzyl, or aryl;
R⁷ and R⁸ are each independently H, C₁₋₈-alkyl, (C₀₋₈-alkyl)-(C₃₋₆-cycloalkyl), phenyl, benzyl, or aryl; or R⁷ and R⁸ can be taken together with the atom connecting them to form a 3 to 7 membered heterocycloalkyl or heteroaryl ring;
R⁹ is C₁₋₄ alkyl, (C₀₋₄alkyl)aryl, (C₀₋₄alkyl)-(C₃₋₆-cycloalkyl), (C₀₋₄alkyl)-heterocylcle.

In one embodiment, W is

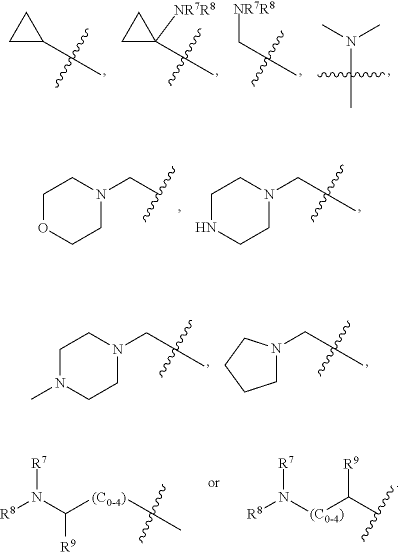

In another embodiment, representative compounds include those of formula:

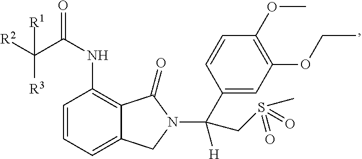

or a pharmaceutically acceptable salt, solvate, hydrate, clathrate, stereoisomer, or prodrug thereof, wherein:
R¹, R², and R³ are independently H or C₁₋₈-alkyl, with the proviso that at least one of R¹, R², and R³ is not H.

Still other PDE4 modulators include, but are not limited to, isoindoline compounds disclosed in U.S. publication no. 2006/0025457A1, published Feb. 2, 2006, which is incorporated herein by reference. Representative compounds include those listed in Table 1 below, or a pharmaceutically acceptable salt, solvate, hydrate, clathrate, stereoisomer, or prodrug thereof:

TABLE 1

| No. | Structure |
|---|---|
| 1 | 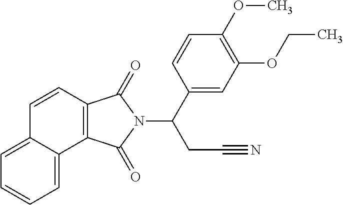 [SMILES: CCOc1cc(C(CC#N)N2C(=O)c3ccc4ccccc4c3C2=O)ccc1OC] |
| 2 | 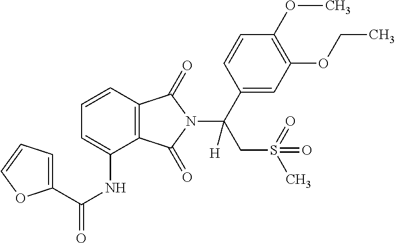 [SMILES: CCOc1cc(C(CS(C)(=O)=O)N2C(=O)c3cccc(NC(=O)c4ccco4)c3C2=O)ccc1OC] |
| 3 | 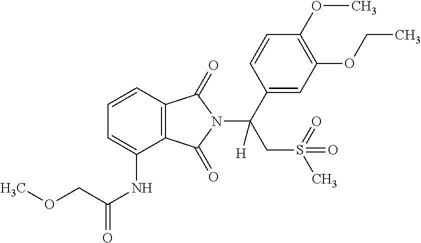 [SMILES: CCOc1cc(C(CS(C)(=O)=O)N2C(=O)c3cccc(NC(=O)COC)c3C2=O)ccc1OC] |
| 4 | 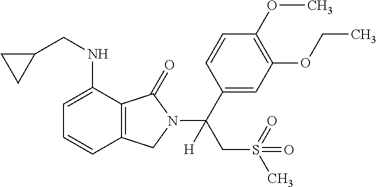 [SMILES: CCOc1cc(C(CS(C)(=O)=O)N2Cc3cccc(NCC4CC4)c3C2=O)ccc1OC] |
| 5 | 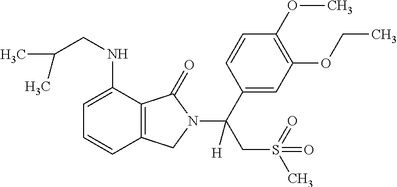 [SMILES: CCOc1cc(C(CS(C)(=O)=O)N2Cc3cccc(NCC(C)C)c3C2=O)ccc1OC] |
| 6 | 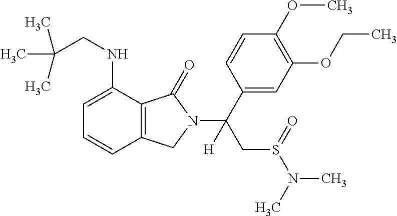 [SMILES: CCOc1cc(C(CS(=O)N(C)C)N2Cc3cccc(NCC(C)(C)C)c3C2=O)ccc1OC] |
| 7 | 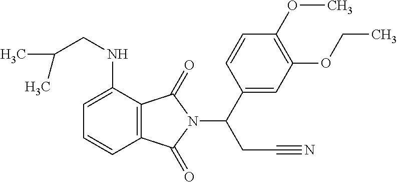 [SMILES: CCOc1cc(C(CC#N)N2C(=O)c3cccc(NCC(C)C)c3C2=O)ccc1OC] |
| 8 | 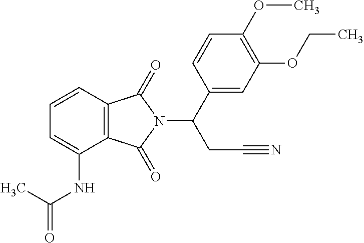 [SMILES: CCOc1cc(C(CC#N)N2C(=O)c3cccc(NC(C)=O)c3C2=O)ccc1OC] |
| 9 | 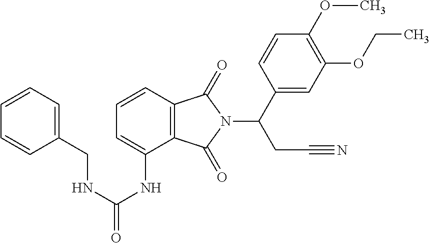 [SMILES: CCOc1cc(C(CC#N)N2C(=O)c3cccc(NC(=O)NCc4ccccc4)c3C2=O)ccc1OC] |
| 10 | 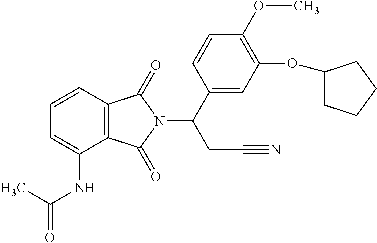 [SMILES: COc1ccc(C(CC#N)N2C(=O)c3cccc(NC(C)=O)c3C2=O)cc1OC1CCCC1] |
| 11 | 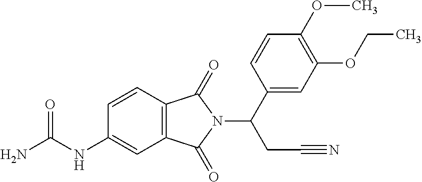 [SMILES: CCOc1cc(C(CC#N)N2C(=O)c3ccc(NC(N)=O)cc3C2=O)ccc1OC] |
| 12 | 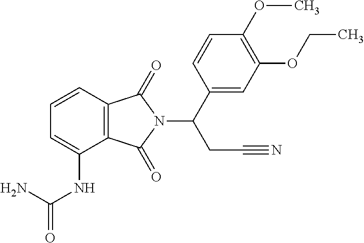 [SMILES: CCOc1cc(C(CC#N)N2C(=O)c3cccc(NC(N)=O)c3C2=O)ccc1OC] |
| 13 | 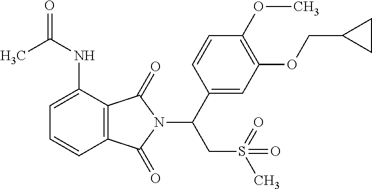 [SMILES: COc1ccc(C(CS(C)(=O)=O)N2C(=O)c3cccc(NC(C)=O)c3C2=O)cc1OCC1CC1] |
| 14 | 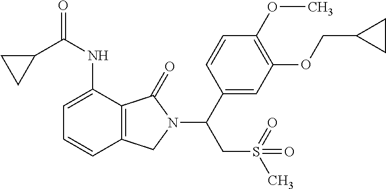 [SMILES: COc1ccc(C(CS(C)(=O)=O)N2Cc3cccc(NC(=O)C4CC4)c3C2=O)cc1OCC1CC1] |
| 15 | 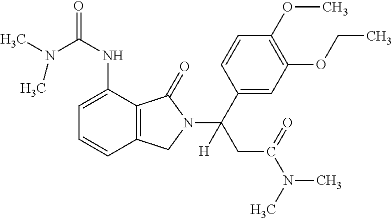 [SMILES: CCOc1cc(C(CC(=O)N(C)C)N2Cc3cccc(NC(=O)N(C)C)c3C2=O)ccc1OC] |
| 16 | 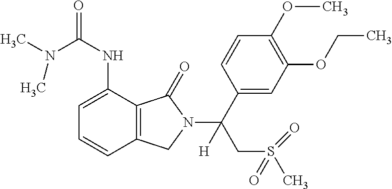 [SMILES: CCOc1cc(C(CS(C)(=O)=O)N2Cc3cccc(NC(=O)N(C)C)c3C2=O)ccc1OC] |

In another embodiment, also provided herein are 2-[1-(3-ethoxy-4-methoxyphenyl)-2-methylsulfonylethyl]-4,5-dinitroisoindoline-1,3-dione, or a pharmaceutically acceptable salt, solvate, hydrate, clathrate, stereoisomer, or prodrug thereof. In one embodiment, provided herein is a hydrochloride salt of 2-[1-(3-ethoxy-4-methoxyphenyl)-2-methylsulfonylethyl]-4,5-dinitroisoindoline-1,3-dione.

Still other PDE4 modulators include, but are not limited to, isoindoline compounds disclosed in U.S. Pat. No. 7,244,259, which is incorporated herein by reference. Representative compounds include cyclopropanecarboxylic acid {2-[1-(3-ethoxy-4-methoxy-phenyl)-2-[1,3,4]oxadiazol-2-yl-ethyl]-3-oxo-2,3-dihydro-1H-isoindol-4-yl}-amide, which has the following chemical structure, or a pharmaceutically acceptable salt, solvate, hydrate, clathrate, stereoisomer, or prodrug thereof:

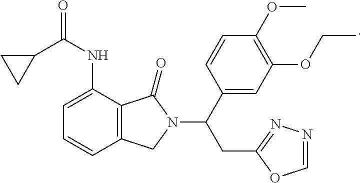 [SMILES: CCOc1cc(C(Cc2nnco2)N2Cc3cccc(NC(=O)C4CC4)c3C2=O)ccc1OC]

Still other PDE4 modulators include, but are not limited to, N-alkyl-hydroxamic acid-isoindolyl compounds disclosed in U.S. Pat. No. 6,911,464, which is incorporated herein by reference. Representative compounds include those of formula:

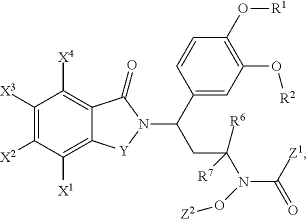

or a pharmaceutically acceptable salt, solvate, hydrate, clathrate, stereoisomer, or prodrug thereof, wherein:
Y is —C(O)—, —CH₂, —CH₂C(O)— or SO₂;
R¹ and R² are independently C₁₋₈-alkyl, CF₂H, CF₃, CH₂CHF₂, cycloalkyl, or (C₁₋₈-alkyl)cycloalkyl;
Z¹ is H, C₁₋₆-alkyl, —NH₂—NR³R⁴ or OR⁵;
Z² is H or C(O)R⁵;
X¹, X², X³ and X⁴ are each independently H, halogen, NO₂, OR₃, CF₃, C₁₋₆-alkyl, (C₀₋₄alkyl)-(C₃₋₆-cycloalkyl), (C₀₋₄-alkyl)-N—(R⁸R⁹), (C₀₋₄-alkyl)-NHC(O)—(R⁸), (C₀₋₄-alkyl)-NHC(O)CH(R⁸)(R⁹), (C₀₋₄-alkyl)-NHC(O)N(R⁸R⁹), (C₀₋₄-alkyl)-NHC(O)O(R), (C₀₋₄-alkyl)-O—R⁸, (C₀₋₄-alkyl)-imidazolyl, (C₀₋₄-alkyl)-pyrrolyl, (C₀₋₄-alkyl)oxadiazolyl, (C₀₋₄-alkyl)-triazolyl or (C₀₋₄-alkyl)-heterocycle;
R³, R⁴, and R⁵ are each independently H, C₁₋₆-alkyl, O—C₁₋₆-alkyl, phenyl, benzyl, or aryl;
R⁶ and R⁷ are independently H or C₁₋₆-alkyl; and
R⁸ and R⁹ are each independently H, C₁₋₉-alkyl, C₃₋₆-cycloalkyl, (C₁₋₆-alkyl)-(C₃₋₆cycloalkyl), (C₀₋₆-alkyl)-N(R⁴R⁵), (C₁₋₆-alkyl)-OR⁵, phenyl, benzyl, aryl, piperidinyl, piperizinyl, pyrolidinyl, morpholino, or C₃₋₇-heterocycloalkyl.

Still other PDE4 modulators include, but are not limited to, diphenylethylene compounds disclosed in U.S. Pat. No. 7,312,241, which is incorporated herein by reference. Representative compounds include those of formula:

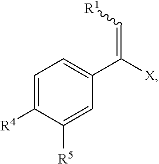

or a pharmaceutically acceptable salt, solvate, hydrate, clathrate, stereoisomer, or prodrug thereof, wherein:
R¹ is —CN, lower alkyl, —COOH, —C(O)—N(R₉)₂, —C(O)-lower alkyl, —C(O)-benzyl, —C(O)O-lower alkyl, —C(O)O-benzyl;
R⁴ is —H, —NO₂, cyano, substituted or unsubstituted lower alkyl, substituted or unsubstituted alkoxy, halogen, —OH, —C(O)(R₁₀)₂, —COOH, —NH₂, or —OC(O)—N(R₁₀)₂;
R⁵ is substituted or unsubstituted lower alkyl, substituted or unsubstituted alkoxy, or substituted or unsubstituted alkenyl;
X is substituted or unsubstituted phenyl, substituted or unsubstituted pyridine, substituted or unsubstituted pyrrolidine, substituted or unsubstituted imidizole, substituted or unsubstituted naphthalene, substituted or unsubstituted thiophene, or substituted or unsubstituted cycloalkyl; each occurrence of R⁹ is independently —H or substituted or unsubstituted lower alkyl; and each occurrence of R¹⁰ is independently —H or substituted or unsubstituted lower alkyl.

In another embodiment, representative compounds include those of formula:

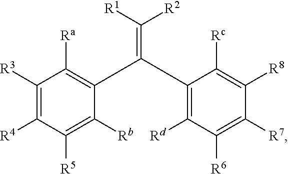

or a pharmaceutically acceptable salt, solvate, hydrate, clathrate, stereoisomer, or prodrug thereof, wherein:
R¹ and R² are independently —H, —CN, substituted or unsubstituted lower alkyl, substituted or unsubstituted alkenyl, substituted or unsubstituted alkynyl, —COOH, —C(O)-lower alkyl, —C(O)O-lower alkyl, —C(O)—N(R₉)₂, substituted or unsubstituted aryl, or substituted or unsubstituted heterocycle;
each occurrence of R^{a}, R^{b}, R^{c} and R^{d} is independently —H, substituted or unsubstituted lower alkyl, substituted or unsubstituted aryl, substituted or unsubstituted heterocycle, substituted or unsubstituted cycloalkyl, substituted or unsubstituted alkoxy, halogen, cyano, —NO₂, —OH, —OPO(OH)₂, —N(R⁹)₂, —OC(O)—R¹⁰, —OC(O)—R¹⁰—N(R¹⁰)₂, —C(O)N(R¹⁰)₂, —NHC(O)—R¹⁰, —NHS(O)₂—R¹⁰, —S(O)₂—R¹⁰, —NHC(O)NH—R¹⁰, —NHC(O)N(R¹⁰)₂, —NHC(O)NHSO₂—R¹⁰, —NHC(O)—R¹⁰—N(R¹⁰)₂, —NHC(O)CH(R¹⁰)(N(R⁹)₂) or —NHC(O)—R¹⁰—NH₂;
R³ is —H, substituted or unsubstituted lower alkyl, substituted or unsubstituted aryl, substituted or unsubstituted heterocycle, substituted or unsubstituted cycloalkyl, substituted or unsubstituted alkoxy, halogen, cyano, —NO₂, —OH, —OPO(OH)₂, —N(R⁹)₂, —OC(O)—R¹⁰, —OC(O)—R¹⁰—N(R¹⁰)₂, —C(O)N(R¹⁰)₂, —NHC(O)—R¹⁰, —NHS(O)₂—R¹⁰, —S(O)₂—R¹⁰, —NHC(O)NH—R¹⁰, —NHC(O)N(R¹⁰)₂, —NHC(O)NHSO₂—R¹⁰, —NHC(O)—R¹⁰—N(R¹⁰)₂, —NHC(O)CH(R¹⁰)(N(R⁹)₂) or —NHC(O)—R¹⁰—NH₂; or R³ with either R^{a} or with R⁴, together form —O—C(R¹⁶R¹⁷)—O— or —O—(C(R¹⁶R¹⁷))₂—O—;
R⁴ is —H, substituted or unsubstituted lower alkyl, substituted or unsubstituted aryl, substituted or unsubstituted heterocycle, substituted or unsubstituted cycloalkyl, substituted or unsubstituted alkoxy, halogen, cyano, —NO₂, —OH, —OPO(OH)₂, —N(R⁹)₂, —OC(O)—R¹⁰, —OC(O)—R¹⁰—N(R¹⁰)₂, —C(O)N(R¹⁰)₂, —NHC(O)—R¹⁰, —NHS(O)₂—R¹⁰, —S(O)₂—R¹⁰, —NHC(O)NH—R¹⁰, —NHC(O)N(R¹⁰)₂, —NHC(O)NHSO₂—R¹⁰, —NHC(O)—R¹⁰—N(R¹⁰)₂, —NHC(O)CH(R¹⁰)(N(R⁹)₂) or —NHC(O)—R¹⁰—NH₂;
R⁵ is —H, substituted or unsubstituted lower alkyl, substituted or unsubstituted aryl, substituted or unsubstituted heterocycle, substituted or unsubstituted cycloalkyl, substituted or unsubstituted alkoxy, halogen, cyano, —NO₂, —OH, —OPO(OH)₂, —N(R⁹)₂, —OC(O)—R¹⁰, —OC(O)—R¹⁰—N(R¹⁰)₂, —C(O)N(R¹⁰)₂, —NHC(O)—R¹⁰, —NHS(O)₂—R¹⁰, —S(O)₂—R¹⁰, —NHC(O)NH—R¹⁰, —NHC(O)N(R¹⁰)₂, —NHC(O)NHSO₂—R¹⁰, —NHC(O)—R¹⁰—N(R¹⁰)₂, —NHC(O)CH(R¹⁰)(N(R⁹)₂) or —NHC(O)—R¹⁰—NH₂;
R⁶ is —H, substituted or unsubstituted lower alkyl, substituted or unsubstituted aryl, substituted or unsubstituted heterocycle, substituted or unsubstituted cycloalkyl, substituted or unsubstituted alkoxy, halogen, cyano, —NO₂, —OH, —OPO(OH)₂, —N(R⁹)₂, —OC(O)—R¹⁰, —OC(O)—R¹⁰—N(R¹⁰)₂, —C(O)N(R¹⁰)₂, —NHC(O)—R¹⁰, —NHS(O)₂—R¹⁰, —S(O)₂—R¹⁰, —NHC(O)NH—R¹⁰, —NHC(O)N(R¹⁰)₂, —NHC(O)NHSO₂—R¹⁰, —NHC(O)—R¹⁰—N(R¹⁰)₂, —NHC(O)CH(R¹⁰)(N(R⁹)₂) or —NHC(O)—R¹⁰—NH₂;
R⁷ is —H, substituted or unsubstituted lower alkyl, substituted or unsubstituted aryl, substituted or unsubstituted heterocycle, substituted or unsubstituted cycloalkyl, substituted or unsubstituted alkoxy, halogen, cyano, —NO₂, —OH, —OPO(OH)₂, —N(R⁹)₂, —OC(O)—R¹⁰, —OC(O)—R¹⁰—N(R¹⁰)₂, —C(O)N(R¹⁰)₂, —NHC(O)—R¹⁰, —NHS(O)₂—R¹⁰, —S(O)₂—R¹⁰, —NHC(O)NH—R¹⁰, —NHC(O)N(R¹⁰)₂, —NHC(O)NHSO₂—R¹⁰, —NHC(O)—R¹⁰—N(R¹⁰)₂, —NHC(O)CH(R¹⁰)(N(R⁹)₂) or —NHC(O)—R¹⁰—NH₂;
R⁸ is —H, substituted or unsubstituted lower alkyl, substituted or unsubstituted aryl, substituted or unsubstituted heterocycle, substituted or unsubstituted cycloalkyl, substituted or unsubstituted alkoxy, halogen, cyano, —NO₂, —OH, —OPO(OH)₂, —N(R⁹)₂, —OC(O)—R¹⁰, —OC(O)—R¹⁰—N(R¹⁰)₂, —C(O)N(R¹⁰)₂, —NHC(O)—R¹⁰, —NHS(O)₂—R¹⁰, —S(O)₂—R¹⁰, —NHC(O)NH—R¹⁰, —NHC(O)N(R¹⁰)₂, —NHC(O)NHSO₂—R¹⁰, —NHC(O)—R¹⁰—N(R¹⁰)₂, —NHC(O)CH(R¹⁰)(N(R⁹)₂) or —NHC(O)—R¹⁰—NH₂, or R⁸ with either Re or with R⁷, together form —O—C(R¹⁶R¹⁷)—O— or —O—(C(R¹⁶R¹⁷))₂—O—;
each occurrence of R⁹ is independently —H, substituted or unsubstituted lower alkyl, or substituted or unsubstituted cycloalkyl;
each occurrence of R¹⁰ is independently substituted or unsubstituted lower alkyl, substituted or unsubstituted cycloalkyl, substituted or unsubstituted aryl, substituted or unsubstituted lower hydroxyalkyl, or R¹⁰ and a nitrogen to which it is attached form a substituted or unsubstituted heterocycle; or R¹⁰ is —H where appropriate; and
each occurrence of R¹⁶ and R¹⁷ is independently —H or halogen.

In another embodiment, provided herein is 3-(3,4-dimethoxyphenyl)-3-(1-oxo-1,3-dihydroisoindol-2-yl)propionic acid methyl ester:

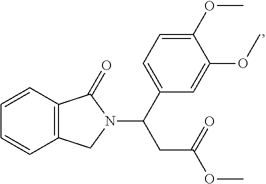 [SMILES: COC(=O)CC(c1ccc(OC)c(OC)c1)N1Cc2ccccc2C1=O]

or a pharmaceutically acceptable salt, solvate, hydrate, clathrate, stereoisomer, or prodrug thereof.

In one embodiment, provided herein are 2-[1-(3-ethoxy-4-methoxyphenyl)-2-methylsulfonylethyl]-4-acetylaminoisoindoline-1,3-dione and cyclopropyl-N-{2-[1-(3-ethoxy-4-methoxyphenyl)-2-(methylsulfonyl)ethyl]-3-oxoisoindolin-4-yl}carboxamide, which respectively have the following structures:

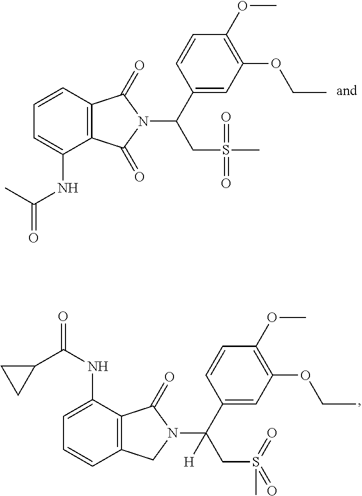 [SMILES: CCOc1cc(C(CS(C)(=O)=O)N2C(=O)c3cccc(NC(C)=O)c3C2=O)ccc1OC ; CCOc1cc(C(CS(C)(=O)=O)N2Cc3cccc(NC(=O)C4CC4)c3C2=O)ccc1OC]

or a pharmaceutically acceptable salt, solvate, hydrate, clathrate, stereoisomer, or prodrug thereof. In another embodiment, stereoisomers of these compounds are also encompassed.

Compounds provided herein can either be commercially purchased or prepared according to the methods described in the patents or patent publications disclosed herein. Further, optically pure compositions can be asymmetrically synthesized or resolved using known resolving agents or chiral columns as well as other standard synthetic organic chemistry techniques.

Various PDE4 modulators contain one or more chiral centers, and can exist as racemic mixtures of enantiomers or mixtures of diastereomers. In one embodiment, provided herein is the use of stereomerically pure forms of such compounds, as well as the use of mixtures of those forms. For example, mixtures comprising equal or unequal amounts of the enantiomers of PDE4 modulators may be used in methods and compositions provided herein. The purified (R) or (S) enantiomers of the specific compounds disclosed herein may be used substantially free of its other enantiomer.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

### 4.4. METHODS OF TREATMENT

The PDE4 modulator provided herein, e.g., Compound A, Compound A1, Compound A2, Compound B, Compound B1, Compound C, or a pharmaceutically acceptable salt, steroisomer, solvate, hydrate, clathrate, or prodrug thereof, can be used in methods for treating, preventing, and/or managing atherosclerosis. Provided herein are methods for treating, preventing and/or managing atherosclerosis, comprising administering to a subject in need thereof an effective amount of a PDE4 modulator provided herein, e.g., Compound A, Compound A1, Compound A2, Compound B, Compound B1, Compound C, or a pharmaceutically acceptable salt, stereoisomer, solvate, hydrate, clathrate, or prodrug thereof.

In some embodiments, provided herein is a PDE4 modulator provided herein, e.g., Compound A, Compound A1, Compound A2, Compound B, Compound B1, Compound C, or a pharmaceutically acceptable salt, steroisomer, solvate, hydrate, clathrate, or prodrug thereof, which can be used in a method for treating, preventing, and/or managing an atherosclerotic cardiovascular disease.

In some embodiments, provided herein is a method for treating, preventing and/or managing an atherosclerotic cardiovascular disease, comprising administering to a subject in need thereof an effective amount of a PDE4 modulator provided herein, e.g., Compound A, Compound A1, Compound A2, Compound B, Compound B1, Compound C, or a pharmaceutically acceptable salt, stereoisomer, solvate, hydrate, clathrate, or prodrug thereof.

In some embodiments, provided herein is a method for treating and/or preventing one or more symptoms associated with atherosclerosis, comprising administering to a subject in need thereof an effective amount of a PDE4 modulator provided herein, e.g., Compound A, Compound A1, Compound A2, Compound B, Compound B1, Compound C, or a pharmaceutically acceptable salt, stereoisomer, solvate, hydrate, clathrate, or prodrug thereof. In some embodiments a PDE4 modulator provided herein e.g., Compound A, Compound A1, Compound A2, Compound B, Compound B1, Compound C, or a pharmaceutically acceptable salt, stereoisomer, solvate, hydrate, clathrate, or prodrug thereof, can be used in a method for treating and/or preventing one or more symptoms associated with atherosclerosis.

In other embodiments, provided herein are methods for treating, preventing and/or managing atherosclerosis, comprising administering to a subject in need thereof an effective amount of N-(2-(1-(3-ethoxy-4-methoxyphenyl)-2-(methylsulfonyl)ethyl)-1,3-dioxoisoindolin-4-yl)acetamide, or a pharmaceutically acceptable salt, solvate, hydrate, clathrate, or prodrug thereof. In other embodiments, an effective amount of N-(2-(1-(3-ethoxy-4-methoxyphenyl)-2-(methylsulfonyl)ethyl)-1,3-dioxoisoindolin-4-yl)acetamide, or a pharmaceutically acceptable salt, solvate, hydrate, clathrate, or prodrug thereof can be used in a method for treating, preventing and/or managing atherosclerosis.

In some embodiments, provided herein is a method for treating, preventing and/or managing an atherosclerotic cardiovascular disease, comprising administering to a subject in need thereof an effective amount of N-(2-(1-(3-ethoxy-4-methoxyphenyl)-2-(methylsulfonyl)ethyl)-1,3-dioxoisoindolin-4-yl)acetamide, or a pharmaceutically acceptable salt, solvate, hydrate, clathrate, or prodrug thereof. In some embodiments, an effective amount of N-(2-(1-(3-ethoxy-4-methoxyphenyl)-2-(methylsulfonyl)ethyl)-1,3-dioxoisoindolin-4-yl)acetamide, or a pharmaceutically acceptable salt, solvate, hydrate, clathrate, or prodrug thereof can be used in a method for treating, preventing and/or managing an atherosclerotic cardiovascular disease

In some embodiments, provided herein is a method for treating and/or preventing one or more symptoms associated with atherosclerosis, comprising administering to a subject in need thereof an effective amount of N-(2-(1-(3-ethoxy-4-methoxyphenyl)-2-(methylsulfonyl)ethyl)-1,3-dioxoisoindolin-4-yl)acetamide, or a pharmaceutically acceptable salt, solvate, hydrate, clathrate, or prodrug thereof. In some embodiments, an effective amount of N-(2-(1-(3-ethoxy-4-methoxyphenyl)-2-(methylsulfonyl)ethyl)-1,3-dioxoisoindolin-4-yl)acetamide, or a pharmaceutically acceptable salt, solvate, hydrate, clathrate, or prodrug thereof can be used in a method for treating and/or preventing one or more symptoms associated with atherosclerosis.

In other embodiments, provided herein are methods for treating, preventing and/or managing atherosclerosis, comprising administering to a subject in need thereof an effective amount of (S)—N-(2-(1-(3-ethoxy-4-methoxyphenyl)-2-(methylsulfonyl)ethyl)-1,3-dioxoisoindolin-4-yl)acetamide, or a pharmaceutically acceptable salt, solvate, hydrate, clathrate, or prodrug thereof. In other embodiments, an effective amount of (S)—N-(2-(1-(3-ethoxy-4-methoxyphenyl)-2-(methylsulfonyl)ethyl)-1,3-dioxoisoindolin-4-yl)acetamide, or a pharmaceutically acceptable salt, solvate, hydrate, clathrate, or prodrug thereof can be used in methods for treating, preventing and/or managing atherosclerosis, comprising administering to a subject in need thereof an effective amount of (S)—N-(2-(1-(3-ethoxy-4-methoxyphenyl)-2-(methylsulfonyl)ethyl)-1,3-dioxoisoindolin-4-yl)acetamide.

In some embodiments, provided herein is a method for treating, preventing and/or managing an atherosclerotic cardiovascular disease, comprising administering to a subject in need thereof an effective amount of (S)—N-(2-(1-(3-ethoxy-4-methoxyphenyl)-2-(methylsulfonyl)ethyl)-1,3-dioxoisoindolin-4-yl)acetamide, or a pharmaceutically acceptable salt, solvate, hydrate, clathrate, or prodrug thereof. In some embodiments, an effective amount of (S)—N-(2-(1-(3-ethoxy-4-methoxyphenyl)-2-(methylsulfonyl)ethyl)-1,3-dioxoisoindolin-4-yl)acetamide, or a pharmaceutically acceptable salt, solvate, hydrate, clathrate, or prodrug thereof can be used in a method for treating, preventing and/or managing an atherosclerotic cardiovascular disease.

In some embodiments, provided herein is a method for treating and/or preventing one or more symptoms associated with atherosclerosis, comprising administering to a subject in need thereof an effective amount of (S)—N-(2-(1-(3-ethoxy-4-methoxyphenyl)-2-(methylsulfonyl)ethyl)-1,3-dioxoisoindolin-4-yl)acetamide, or a pharmaceutically acceptable salt, solvate, hydrate, clathrate, or prodrug thereof. In some embodiments, an effective amount of (S)—N-(2-(1-(3-ethoxy-4-methoxyphenyl)-2-(methylsulfonyl)ethyl)-1,3-dioxoisoindolin-4-yl)acetamide, or a pharmaceutically acceptable salt, solvate, hydrate, clathrate, or prodrug thereof can be used in a method for treating and/or preventing one or more symptoms associated with atherosclerosis.

In other embodiments, provided herein are methods for treating, preventing and/or managing atherosclerosis, comprising administering to a subject in need thereof an effective amount of (R)—N-(2-(1-(3-ethoxy-4-methoxyphenyl)-2-(methylsulfonyl)ethyl)-1,3-dioxoisoindolin-4-yl)acetamide, or a pharmaceutically acceptable salt, solvate, hydrate, clathrate, or prodrug thereof. In other embodiments, an effective amount of (R)—N-(2-(1-(3-ethoxy-4-methoxyphenyl)-2-(methylsulfonyl)ethyl)-1,3-dioxoisoindolin-4-yl)acetamide, or a pharmaceutically acceptable salt, solvate, hydrate, clathrate, or prodrug thereof can be used in methods for treating, preventing and/or managing atherosclerosis.

In some embodiments, provided herein is a method for treating, preventing and/or managing an atherosclerotic cardiovascular disease, comprising administering to a subject in need thereof an effective amount of (R)—N-(2-(1-(3-ethoxy-4-methoxyphenyl)-2-(methylsulfonyl)ethyl)-1,3-dioxoisoindolin-4-yl)acetamide, or a pharmaceutically acceptable salt, solvate, hydrate, clathrate, or prodrug thereof. In some embodiments, an effective amount of (R)—N-(2-(1-(3-ethoxy-4-methoxyphenyl)-2-(methylsulfonyl)ethyl)-1,3-dioxoisoindolin-4-yl)acetamide, or a pharmaceutically acceptable salt, solvate, hydrate, clathrate, or prodrug thereof can be used in a method for treating, preventing and/or managing an atherosclerotic cardiovascular disease.

In some embodiments, provided herein is a method for treating and/or preventing one or more symptoms associated with atherosclerosis, comprising administering to a subject in need thereof an effective amount of (R)—N-(2-(1-(3-ethoxy-4-methoxyphenyl)-2-(methylsulfonyl)ethyl)-1,3-dioxoisoindolin-4-yl)acetamide, or a pharmaceutically acceptable salt, solvate, hydrate, clathrate, or prodrug thereof. In some embodiments, an effective amount of (R)—N-(2-(1-(3-ethoxy-4-methoxyphenyl)-2-(methylsulfonyl)ethyl)-1,3-dioxoisoindolin-4-yl)acetamide, or a pharmaceutically acceptable salt, solvate, hydrate, clathrate, or prodrug thereof can be used in a method for treating and/or preventing one or more symptoms associated with atherosclerosis.

In other embodiments, provided herein are methods for treating, preventing and/or managing atherosclerosis, comprising administering to a subject in need thereof an effective amount of N-(2-(1-(3-ethoxy-4-methoxyphenyl)-2-(methylsulfonyl)ethyl)-3-oxoisoindolin-4-yl)cyclopropanecarboxamide, or a pharmaceutically acceptable salt, solvate, hydrate, clathrate, or prodrug thereof. In other embodiments, an effective amount of N-(2-(1-(3-ethoxy-4-methoxyphenyl)-2-(methylsulfonyl)ethyl)-3-oxoisoindolin-4-yl)cyclopropanecarboxamide, or a pharmaceutically acceptable salt, solvate, hydrate, clathrate, or prodrug thereof can be used in methods for treating, preventing and/or managing atherosclerosis.

In some embodiments, provided herein is a method for treating, preventing and/or managing an atherosclerotic cardiovascular disease, comprising administering to a subject in need thereof an effective amount of N-(2-(1-(3-ethoxy-4-methoxyphenyl)-2-(methylsulfonyl)ethyl)-3-oxoisoindolin-4-yl)cyclopropanecarboxamide, or a pharmaceutically acceptable salt, solvate, hydrate, clathrate, or prodrug thereof. In some embodiments, an effective amount of N-(2-(1-(3-ethoxy-4-methoxyphenyl)-2-(methylsulfonyl)ethyl)-3-oxoisoindolin-4-yl)cyclopropanecarboxamide, or a pharmaceutically acceptable salt, solvate, hydrate, clathrate, or prodrug thereof can be used in a method for treating, preventing and/or managing an atherosclerotic cardiovascular disease.

In some embodiments, provided herein is a method for treating and/or preventing one or more symptoms associated with atherosclerosis, comprising administering to a subject in need thereof an effective amount of N-(2-(1-(3-ethoxy-4-methoxyphenyl)-2-(methylsulfonyl)ethyl)-3-oxoisoindolin-4-yl)cyclopropanecarboxamide, or a pharmaceutically acceptable salt, solvate, hydrate, clathrate, or prodrug thereof. In some embodiments, an effective amount of N-(2-(1-(3-ethoxy-4-methoxyphenyl)-2-(methylsulfonyl)ethyl)-3-oxoisoindolin-4-yl)cyclopropanecarboxamide, or a pharmaceutically acceptable salt, solvate, hydrate, clathrate, or prodrug thereof can be used in a method for treating and/or preventing one or more symptoms associated with atherosclerosis.

In other embodiments, provided herein are methods for treating, preventing and/or managing atherosclerosis, comprising administering to a subject in need thereof an effective amount of (S)—N-(2-(1-(3-ethoxy-4-methoxyphenyl)-2-(methylsulfonyl)ethyl)-3-oxoisoindolin-4-yl)cyclopropanecarboxamide, or a pharmaceutically acceptable salt, solvate, hydrate, clathrate, or prodrug thereof. In other embodiments, an effective amount of (S)—N-(2-(1-(3-ethoxy-4-methoxyphenyl)-2-(methylsulfonyl)ethyl)-3-oxoisoindolin-4-yl)cyclopropanecarboxamide, or a pharmaceutically acceptable salt, solvate, hydrate, clathrate, or prodrug thereof can be used in methods for treating, preventing and/or managing atherosclerosis.

In some embodiments, provided herein is a method for treating, preventing and/or managing an atherosclerotic cardiovascular disease, comprising administering to a subject in need thereof an effective amount of (S)—N-(2-(1-(3-ethoxy-4-methoxyphenyl)-2-(methylsulfonyl)ethyl)-3-oxoisoindolin-4-yl)cyclopropanecarboxamide, or a pharmaceutically acceptable salt, solvate, hydrate, clathrate, or prodrug thereof. In some embodiments, an effective amount of (S)—N-(2-(1-(3-ethoxy-4-methoxyphenyl)-2-(methylsulfonyl)ethyl)-3-oxoisoindolin-4-yl)cyclopropanecarboxamide, or a pharmaceutically acceptable salt, solvate, hydrate, clathrate, or prodrug thereof can be used in a method for treating, preventing and/or managing an atherosclerotic cardiovascular disease.

In some embodiments, provided herein is a method for treating and/or preventing one or more symptoms associated with atherosclerosis, comprising administering to a subject in need thereof an effective amount of (S)—N-(2-(1-(3-ethoxy-4-methoxyphenyl)-2-(methylsulfonyl)ethyl)-3-oxoisoindolin-4-yl)cyclopropanecarboxamide, or a pharmaceutically acceptable salt, solvate, hydrate, clathrate, or prodrug thereof. In some embodiments, an effective amount of (S)—N-(2-(1-(3-ethoxy-4-methoxyphenyl)-2-(methylsulfonyl)ethyl)-3-oxoisoindolin-4-yl)cyclopropanecarboxamide, or a pharmaceutically acceptable salt, solvate, hydrate, clathrate, or prodrug thereof can be used in a method for treating and/or preventing one or more symptoms associated with atherosclerosis.

In other embodiments, provided herein are methods for treating, preventing and/or managing atherosclerosis, comprising administering to a subject in need thereof an effective amount of 3-(3,4-dimethoxy-phenyl)-3-(1-oxo-1,3-dihydro-isoindol-2-yl)-propionamide, or a pharmaceutically acceptable salt, stereoisomer, solvate, hydrate, clathrate, or prodrug thereof. In other embodiments, an effective amount of 3-(3,4-dimethoxy-phenyl)-3-(1-oxo-1,3-dihydro-isoindol-2-yl)-propionamide, or a pharmaceutically acceptable salt, stereoisomer, solvate, hydrate, clathrate, or prodrug thereof can be used in methods for treating, preventing and/or managing atherosclerosis.

In some embodiments, provided herein is a method for treating, preventing and/or managing an atherosclerotic cardiovascular disease, comprising administering to a subject in need thereof an effective amount of 3-(3,4-dimethoxy-phenyl)-3-(1-oxo-1,3-dihydro-isoindol-2-yl)-propionamide, or a pharmaceutically acceptable salt, stereoisomer, solvate, hydrate, clathrate, or prodrug thereof. In some embodiments, an effective amount of 3-(3,4-dimethoxy-phenyl)-3-(1-oxo-1,3-dihydro-isoindol-2-yl)-propionamide, or a pharmaceutically acceptable salt, stereoisomer, solvate, hydrate, clathrate, or prodrug thereof can be used in a method for treating, preventing and/or managing an atherosclerotic cardiovascular disease.

In some embodiments, provided herein is a method for treating and/or preventing one or more symptoms associated with atherosclerosis, comprising administering to a subject in need thereof an effective amount of 3-(3,4-dimethoxy-phenyl)-3-(1-oxo-1,3-dihydro-isoindol-2-yl)-propionamide, or a pharmaceutically acceptable salt, stereoisomer, solvate, hydrate, clathrate, or prodrug thereof. In some embodiments, an effective amount of 3-(3,4-dimethoxy-phenyl)-3-(1-oxo-1,3-dihydro-isoindol-2-yl)-propionamide, or a pharmaceutically acceptable salt, stereoisomer, solvate, hydrate, clathrate, or prodrug thereof can be used in a method for treating and/or preventing one or more symptoms associated with atherosclerosis.

In some embodiments, methods provided herein comprise administering a PDE4 modulator provided herein, e.g., Compound A, Compound A1, Compound A2, Compound B, Compound B1, Compound C, or a pharmaceutically acceptable salt, stereoisomer, solvate, hydrate, clathrate, or prodrug thereof, after the onset of symptoms of the atherosclerosis or atherosclerotic cardiovascular disease. In some embodiments, methods provided herein comprise administering either one of Compound A1 or Compound B1, substantially free of its (−) enantiomer, or a pharmaceutically acceptable salt, solvate, hydrate, clathrate, or prodrug thereof, after the onset of symptoms of the atherosclerosis or atherosclerotic cardiovascular disease.

In some embodiments, methods provided herein comprise administering a PDE4 modulator as disclosed herein, e.g., Compound A, Compound A1, Compound A2, Compound B, Compound B1, Compound C, or a pharmaceutically acceptable salt, stereoisomer, solvate, hydrate, clathrate, or prodrug thereof, prior to the onset of symptoms of the atherosclerosis or atherosclerotic cardiovascular disease. In some embodiments, methods provided herein comprise administering either one of Compound A1 or Compound B1, substantially free of its (−) enantiomer, or a pharmaceutically acceptable salt, solvate, hydrate, clathrate, or prodrug thereof, prior to the onset of symptoms of the atherosclerosis or atherosclerotic cardiovascular disease.

Symptoms of atherosclerosis or atherosclerotic cardiovascular disease include, but are not limited to, angina, shortness of breath, arrhythmias, sleep problems, lack of energy, fatigue, sudden weakness, numbness, paralysis, trouble in seeing objects, dizziness, loss of consciousness, severe headache, and claudication.

In some embodiments, methods provided herein decrease the intensity of the atherosclerosis or atherosclerotic cardiovascular disease.

In some embodiments, methods provided herein alleviate or prevent the atherosclerosis or atherosclerotic cardiovascular disease.

In some embodiments, provided herein are also methods of predicting response to treatment or management, or effectiveness of prevention, in a subject, comprising obtaining a biological sample from the subject and measuring the presence or absence of a biomarker. The biomarker can be any biomarker associated with PDE4 modulation. In one embodiment, the biological sample is blood, serum, or plasma. In another embodiment, the biological sample is a lesion, e.g., abnormal tissue of an organism. In one embodiment, the biomarker is a chemokine or cytokine. In one embodiment, the subject to be treated exhibits an abnormal (e.g., elevated) level of the biomarker.

### 4.5. COMBINATION THERAPY

In some embodiments, provided herein are methods for combination therapies in which a PDE4 modulator provided herein, e.g., Compound A, Compound A1, Compound A2, Compound B, Compound B1, Compound C, or a pharmaceutically acceptable salt, stereoisomer, solvate, hydrate, clathrate, or prodrug thereof, is used in combination with another therapeutic agent (“second active agent”).

In some embodiments, provided herein are synergistic combinations for treatment of atherosclerosis or atherosclerotic cardiovascular disease. In one embodiment, when administered as a component of such combination therapy, the PDE4 modulator and the other medicament may be synergistic, such that the dose of either or both of the components may be reduced as compared to the dose of either component that would normally be given as a monotherapy. In one embodiment, when administered as a component of such combination therapy, the PDE4 modulator and the other medicament may be additive, such that the dose of each of the components is similar or the same as the dose of either component that would normally be given as a monotherapy.

The PDE4 modulators provided herein can also be used to alleviate adverse or unnamed effects associated with some second active agents, and conversely some second active agents can be used to alleviate adverse or unnamed effects associated with the PDE4 modulators provided herein.

In some embodiments, the combination therapies provided herein include, but are not limited to, the combination of a PDE4 modulator provided herein, e.g., Compound A, Compound A1, Compound A2, Compound B, Compound B1, Compound C, or a pharmaceutically acceptable salt, stereoisomer, solvate, hydrate, clathrate, or prodrug thereof, with one or more of conventional therapies used to treat or prevent atherosclerosis.

In some embodiments, the second active agents may include, but are not limited to, anti-cholesterol agents such as statins (e.g., atorvastatin, fluvastatin, lovastatin, pitavastatin, pravastatin, rosuvastatin, and simvastatin), bile acid sequestrants (e.g., cholestyramine, colesevelam, and colestipol), cholesterol absorption inhibitors (e.g., exetimibe), nicotinc acids (e.g., niacin), and fibric acid derivatives (e.g., fenofibrate and gemfibrozil). In some embodiments, the second active agents can be anti-cholesterol agents such as statins (e.g., atorvastatin, fluvastatin, lovastatin, pitavastatin, pravastatin, rosuvastatin, and simvastatin). In some embodiments, the second active agents can be bile acid sequestrants (e.g., cholestyramine, colesevelam, and colestipol). In some embodiments, the second active agents can be cholesterol absorption inhibitors (e.g., exetimibe)). In some embodiments, the second active agents can be nicotinc acids (e.g., niacin)). In some embodiments, the second active agents can be fibric acid derivatives (e.g., fenofibrate and gemfibrozil).

In some embodiments, the second active agents may include, but are not limited to, high blood pressure/hypertension medications such as diuretics (e.g., spironolactone, triamterene, hydrochlorothiazide, chlorthalidone, furosemide, indapamide, amiloride, metolazone, and combinations thereof), beta blockers (e.g., timolol, caterolol, carvediol, nadolol, propanolol, betaxolol, penbutolol, metoprolol, acebutolol, atenolol, pindolol, bisoprolol, and labetolol), alpha blockers (e.g., doxazosin, terazosin, and prazosin), and ACE inhibitors (e.g., quinapril, ramipril, trandolapril, captopril, benazepril, fosinopril, lisinopril, moexipril, and enalapril). In some embodiments, the second active agents can be high blood pressure/hypertension medications such as diuretics (e.g., spironolactone, triamterene, hydrochlorothiazide, chlorthalidone, furosemide, indapamide, amiloride, metolazone, and combinations thereof). In some embodiments, the second active agents can be high blood pressure/hypertension medications beta blockers (e.g., timolol, caterolol, carvediol, nadolol, propanolol, betaxolol, penbutolol, metoprolol, acebutolol, atenolol, pindolol, bisoprolol, and labetolol), In some embodiments, the second active agents can be high blood pressure/hypertension medications such as alpha blockers (e.g., doxazosin, terazosin, and prazosin), and ACE inhibitors (e.g., quinapril, ramipril, trandolapril, captopril, benazepril, fosinopril, lisinopril, moexipril, and enalapril). In some embodiments, the second active agents can be high blood pressure/hypertension medications such as ACE inhibitors (e.g., quinapril, ramipril, trandolapril, captopril, benazepril, fosinopril, lisinopril, moexipril, and enalapril).

Specific methods provided herein comprise administering either one of Compound A1 or Compound B1, substantially free of its (−) enantiomer, or a pharmaceutically acceptable salt, solvate, hydrate, clathrate, or prodrug thereof, in combination with at least a second active agent or another therapy.

Administration of a PDE4 modulator provided herein and at least one second active agent to a patient can occur simultaneously or sequentially by the same or different routes of administration. The suitability of a particular route of administration employed for a particular second active agent will depend on the second active agent itself (e.g., whether it can be administered topically or orally without decomposition prior to entering the blood stream) and the disease being treated. A particular route of administration for a PDE4 modulator provided herein is oral or topical administration. Particular routes of administration for the second active agents or ingredients of the invention are known to those of ordinary skill in the art. See, e.g., The Merck Manual, 430-431 (17th ed., 1999).

The amount of second active agent administered can be determined based on the specific agent used, the type of disease being treated or managed, the severity and stage of disease and the amount(s) of the compounds of the invention and any optional additional second active agents concurrently administered to the patient. Those of ordinary skill in the art can determine the specific amounts according to conventional procedures known in the art. In the beginning, one can start from the amount of the second active agent that is conventionally used in the therapies and adjust the amount according to the factors described above. See, e.g., Physician's Desk Reference (56^{th} Ed., 2004). Further, the amounts and methods of administration of the second active agents disclosed herein for the treatment, prevention and/or management of cutaneous lupus are disclosed in the literature, e.g., Physician's Desk Reference (56^{th} Ed., 2004), which is incorporated herein by reference.

In some embodiments, a PDE4 modulator provided herein can be used in combination with other types of therapies for atherosclerosis. Examples include, but are not limited to: life style changes such as healthy diet, exercising, maintaining healthy weight, smoking cessation, stress management; and medical procedures and surgery such as angioplasty, coronary artery bypass grafting, and carotid endarterectomy.

### 4.6. PHARMACEUTICAL COMPOSITIONS AND DOSAGE FORMS

Pharmaceutical compositions can be used in the preparation of individual, single unit dosage forms. Pharmaceutical compositions and dosage forms provided herein may comprise a PDE4 modulator, including but not limited to Compound A, Compound A1, Compound A2, Compound B, Compound B1, Compound C, or a pharmaceutically acceptable salt, stereoisomer, solvate, hydrate, clathrate, or prodrug thereof. Pharmaceutical compositions and dosage forms may further comprise one or more carriers, excipients, or diluents.

Single unit dosage forms provided herein are suitable for oral, mucosal (e.g., nasal, sublingual, vaginal, cystic, rectal, preputial, ocular, buccal or aural), parenteral (e.g., subcutaneous, intravenous, bolus injection, intramuscular or intraarterial), topical (e.g., eye drops or other ophthalmic preparations), transdermal or transcutaneous administration to a patient. Non-limiting examples of dosage forms include tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions or a water-in-oil liquid emulsions), solutions and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

The composition, shape and type of dosage forms provided herein will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms provided herein will vary from one another will be readily apparent to those skilled in the art. See, e.g., Remington's Pharmaceutical Sciences, 20th ed., Mack Publishing, Easton Pa. (2000).

Typical pharmaceutical compositions and dosage forms comprise one or more excipients. Suitable excipients are well known to those skilled in the art of pharmacy and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients can be accelerated by some excipients such as lactose or when exposed to water. Active ingredients that comprise primary or secondary amines are particularly susceptible to such accelerated decomposition. Consequently, provided herein are pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions provided herein may comprise excipients that are well known in the art and are listed, for example, in the U.S. Pharmacopeia (USP) 25-NF20 (2002). In general, lactose-free compositions comprise active ingredients, a binder/filler and a lubricant in pharmaceutically compatible and pharmaceutically acceptable amounts. Particular lactose-free dosage forms comprise active ingredients, microcrystalline cellulose, pre-gelatinized starch and magnesium stearate.

Also provided herein are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, N.Y., N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment and use of formulations.

Anhydrous pharmaceutical compositions and dosage forms provided herein may be prepared using anhydrous or low moisture containing ingredients and low moisture or low humidity conditions. Pharmaceutical compositions and dosage forms that comprise lactose and at least one active ingredient that comprise a primary or secondary amine are preferably anhydrous if substantial contact with moisture and/or humidity during manufacturing, packaging and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Non-limiting examples of suitable packaging include hermetically sealed foils, plastics, unit dose containers (e.g., vials), blister packs and strip packs.

Also provided herein are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers or salt buffers. Like the amounts and types of excipients, the amounts and specific types of active ingredients in a dosage form may differ depending on factors such as, but not limited to, the route by which it is to be administered to patients. However, typical dosage forms provided herein comprise either one of (S)—N-(2-(1-(3-ethoxy-4-methoxyphenyl)-2-(methylsulfonyl)ethyl)-1,3-dioxoisoindolin-4-yl)acetamide or (S)—N-(2-(1-(3-ethoxy-4-methoxyphenyl)-2-(methylsulfonyl)ethyl)-3-oxoisoindolin-4-yl)cyclopropanecarboxamide, or a pharmaceutically acceptable salt or solvate thereof in an amount of from about 1 to about 1,000 mg. Typical dosage forms comprise one of Compound A, Compound A1, Compound A2, Compound B, Compound B1, Compound C, or a pharmaceutically acceptable salt, stereoisomer, or solvate thereof in an amount of about 1, 2, 5, 7.5, 10, 12.5, 15, 17.5, 20, 25, 30, 50, 100, 150 or 200 mg. In a particular embodiment, a dosage form comprises one of Compound A, Compound A1, Compound A2, Compound B, Compound B1, or Compound C, in an amount of about 1, 5, 10, 15, 20, 25, 30, 50, 100 or 200 mg.

The magnitude of a therapeutic dose of a particular PDE4 modulator will vary, however, with the nature and severity of the disease or condition, and the route by which the active ingredient is administered. The dose, and perhaps the dose frequency, will also vary according to the age, body weight, and response of the individual subject. Suitable dosing regimens can be readily selected by those skilled in the art with due consideration of such factors.

In general, the recommended daily dose ranges described herein lie within the range of from about 0.1 mg to about 1,000 mg per day, given as a single once-a-day dose or as divided doses throughout a day. More specifically, the daily dose may be administered once, twice, three times, or four times daily in equally divided doses. Specifically, a daily dose range may be from about 1 mg to about 500 mg per day, more specifically, between about 10 mg and about 200 mg per day. Specifically, the daily dose may be administered in 1 mg, 5 mg, 6.25 mg, 10 mg, 15 mg, 20 mg, 25 mg, 30 mg, 50 mg, 100 mg, 200 mg, 300 mg, 400 mg or 500 mg dosage forms (Q.D. or B.I.D.). In managing the subject, the therapy may be initiated at a lower dose, perhaps about 1 mg to about 25 mg, and increased if necessary up to about 200 mg to about 1,000 mg per day as either a single dose or divided doses, depending on the subject's global response. In further embodiments, the daily dose of Compound A, Compound A1, Compound A2, Compound B, Compound B1, or Compound C is from about 0.01 mg to about 100 mg per kg of a body weight of a subject. In some embodiments, the daily dose of the chosen compound is about 1 mg/kg, 5 mg/kg, 6.25 mg/kg, 10 mg/kg, 25 mg/kg or 50 mg/kg. In certain embodiments, the therapeutically effective amount of the first active agent as provided herein is about 1, 5, 10, 25, or 50 mg per kg of a body weight of the subject per day and the therapeutically effective amount of the additional active agent as provided herein is about 1, 5, 10, 25, 50, 100, 200, 500, or 1000 mg per kg of a body weight of the subject per day.

### 4.6.1. Oral Dosage Forms

Pharmaceutical compositions provided herein that are suitable for oral administration can be presented as discrete dosage forms, such as, but not limited to, tablets (e.g., chewable tablets), caplets, capsules, satchet, and liquids (e.g., flavored syrups). Such dosage forms contain predetermined amounts of active ingredients and can be prepared by methods of pharmacy well known to those skilled in the art. See generally, Remington's Pharmaceutical Sciences, 20th ed., Mack Publishing, Easton Pa. (2000).

Typical oral dosage forms provided herein are prepared by combining the active ingredients in an intimate admixture with at least one excipient according to conventional pharmaceutical compounding techniques. Excipients can take a wide variety of forms depending on the form of preparation desired for administration. Non-limiting examples of excipients suitable for use in oral liquid or aerosol dosage forms include water, glycols, oils, alcohols, flavoring agents, preservatives and coloring agents. Non-limiting examples of excipients suitable for use in solid oral dosage forms (e.g., powders, tablets, capsules and caplets) include starches, sugars, micro-crystalline cellulose, diluents, granulating agents, lubricants, binders and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers or both and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Non-limiting examples of excipients that can be used in oral dosage forms provided herein include binders, fillers, disintegrants and lubricants. Non-limiting examples of binders suitable for use in pharmaceutical compositions and dosage forms include corn starch, potato starch or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose and mixtures thereof.

Non-limiting examples of suitable forms of microcrystalline cellulose include the materials sold as AVICEL-PH-101, AVICEL-PH-103 AVICEL RC-581, AVICEL-PH-105 (available from FMC Corporation, American Viscose Division, Avicel Sales, Marcus Hook, Pa.) and mixtures thereof. A specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Non-limiting examples of fillers suitable for use in the pharmaceutical compositions and dosage forms disclosed herein include talc, calcium carbonate (e.g., granules or powder), microcrystalline cellulose, powdered cellulose, dextrates, kaolin, mannitol, silicic acid, sorbitol, starch, pre-gelatinized starch and mixtures thereof. The binder or filler in pharmaceutical compositions is typically present in from about 50 to about 99 weight percent of the pharmaceutical composition or dosage form.

Disintegrants may be used in the compositions provided herein to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. The amount of disintegrant used varies based upon the type of formulation and is readily discernible to those of ordinary skill in the art. Typical pharmaceutical compositions comprise from about 0.5 to about 15 weight percent of disintegrant, preferably from about 1 to about 5 weight percent of disintegrant.

Non-limiting examples of disintegrants that can be used in pharmaceutical compositions and dosage forms provided herein include agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums and mixtures thereof.

Non-limiting examples of lubricants that can be used in pharmaceutical compositions and dosage forms provided herein include calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil and soybean oil), zinc stearate, ethyl oleate, ethyl laureate, agar and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.) and mixtures thereof. If used at all, lubricants are typically used in an amount of less than about 1 weight percent of the pharmaceutical compositions or dosage forms into which they are incorporated.

In one embodiment, a solid oral dosage form provided herein comprises either one of Compound A, Compound A1, Compound B, Compound B1, or Compound C, and anhydrous lactose, microcrystalline cellulose, polyvinylpyrrolidone, stearic acid, colloidal anhydrous silica and gelatin.

### 4.6.2. Delayed Release Dosage Forms

Active ingredients can be administered by controlled release means or by delivery devices that are well known to those of ordinary skill in the art. Non-limiting examples of controlled release means or delivery devices include those described in U.S. Pat. Nos. 3,845,770; 3,916,899; 3,536,809; 3,598,123; and U.S. Pat. Nos. 4,008,719, 5,674,533, 5,059,595, 5,591,767, 5,120,548, 5,073,543, 5,639,476, 5,354,556 and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres or a combination thereof to provide the desired release profile in varying proportions. Suitable controlled-release formulations known to those of ordinary skill in the art, including those described herein, can be readily selected for use with the active ingredients provided herein. Thus, in some embodiments, provided herein are single unit dosage forms suitable for oral administration such as, but not limited to, tablets, capsules, gelcaps and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency and increased patient compliance. In addition, controlled-release formulations can be used to affect the time of onset of action or other characteristics, such as blood levels of the drug and can thus affect the occurrence of side (e.g., adverse) effects.

Most controlled-release formulations are designed to initially release an amount of drug (active ingredient) that promptly produces the desired therapeutic effect and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water or other physiological conditions or compounds.

### 4.6.3. Parenteral Dosage Forms

Parenteral dosage forms can be administered to patients by various routes including, but not limited to, subcutaneous, intravenous (including bolus injection), intramuscular and intraarterial. Because their administration typically bypasses patients' natural defenses against contaminants, parenteral dosage forms are preferably sterile or capable of being sterilized prior to administration to a patient. Non-limiting examples of parenteral dosage forms include solutions ready for injection, dry products ready to be dissolved or suspended in a pharmaceutically acceptable vehicle for injection, suspensions ready for injection and emulsions.

Suitable vehicles that can be used to provide parenteral dosage forms are well known to those skilled in the art. Non-limiting examples of suitable vehicles include Water for Injection USP; aqueous vehicles such as, but not limited to, Sodium Chloride Injection, Ringer's Injection, Dextrose Injection, Dextrose and Sodium Chloride Injection and Lactated Ringer's Injection; water-miscible vehicles such as, but not limited to, ethyl alcohol, polyethylene glycol and polypropylene glycol; and non-aqueous vehicles such as, but not limited to, corn oil, cottonseed oil, peanut oil, sesame oil, ethyl oleate, isopropyl myristate and benzyl benzoate.

Compounds that increase the solubility of one or more of the active ingredients disclosed herein can also be incorporated into the parenteral dosage forms provided herein. For example, cyclodextrin and its derivatives can be used to increase the solubility of either one of (S)—N-(2-(1-(3-ethoxy-4-methoxyphenyl)-2-(methylsulfonyl)ethyl)-1,3-dioxoisoindolin-4-yl)acetamide or (S)—N-(2-(1-(3-ethoxy-4-methoxyphenyl)-2-(methylsulfonyl)ethyl)-3-oxoisoindolin-4-yl)cyclopropanecarboxamide and their derivatives.

### 4.6.4. Topical and Mucosal Dosage Forms

Drugs can be applied locally to the skin and its adnexa or to a variety of mucous membranes. The routes that can be used include nasal, sublingual, vaginal, cystic, rectal, preputial, ocular, buccal or aural. Many dosage forms have been developed to deliver active principles to the site of application to produce local effects. Non-limiting examples of topical and mucosal dosage forms provided herein include sprays, inhalers, aerosols, ointments, creams, gels, pastes, dusting powders, lotions, liniments, poultices, solutions, emulsions, suspensions, eye drops or other ophthalmic preparations or other forms known to one of skill in the art. See, e.g., Remington's Pharmaceutical Sciences, 20^{th} ed., Mack Publishing, Easton Pa. (2000); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms are well known to those skilled in the pharmaceutical arts and depend on the particular tissue to which a given pharmaceutical composition or dosage form will be applied. Non-limiting examples of typical excipients include water, acetone, ethanol, ethylene glycol, propylene glycol, butane-1,3-diol, isopropyl myristate, isopropyl palmitate, mineral oil and mixtures thereof to form solutions, emulsions or gels, which are non-toxic and pharmaceutically acceptable.

Moisturizers such as occlusives, humectants, emollients and protein rejuvenators can also be added to pharmaceutical compositions and dosage forms if desired. Examples of such additional ingredients are well known in the art. See, e.g., Remington's Pharmaceutical Sciences, 20^{th} ed., Mack Publishing, Easton Pa. (2000).

Occlusives are substances that physically block water loss in the stratum corneum. Non-limiting examples of occlusives include petrolatum, lanolin, mineral oil, silicones such as dimethicone, zinc oxide and combinations thereof. Preferably, the occlusives are petrolatum and lanolin, more preferably petrolatum in a minimum concentration of 5%.

Humectants are substances that attract water when applied to the skin and theoretically improve hydration of the stratum corneum. However, the water that is drawn to the skin is water from other cells, not atmospheric water. With this type of moisturizer, evaporation from the skin can continue and actually can make the dryness worse. Non-limiting examples of humectants include glycerin, sorbitol, urea, alpha hydroxy acids, sugars and combinations thereof. Preferably, the humectants are alpha hydroxy acids, such as glycolic acid, lactic acid, malic acid, citric acid and tartaric acid.

Emollients are substances that smooth skin by filling spaces between skin flakes with droplets of oil, and are not usually occlusive unless applied heavily. When combined with an emulsifier, they may help hold oil and water in the stratum corneum. Vitamin E is a common additive, which appears to have no effect, except as an emollient. Likewise, other vitamins, for example, A and D, are also added, but their effect is questionable. Non-limiting examples of emollients include mineral oil, lanolin, fatty acids, cholesterol, squalene, structural lipids and combinations thereof.

Protein rejuvenators are substances that rejuvenate the skin by replenishing essential proteins. Non-limiting examples of protein rejuvenators include collagen, keratin, elastin and combinations thereof.

The pH of a pharmaceutical composition or dosage form may also be adjusted to improve delivery of one or more active ingredients. Similarly, the polarity of a solvent carrier, its ionic strength or tonicity can be adjusted to improve delivery. For example, absorption through the skin can also be enhanced by occlusive dressings, inunction or the use of dimethyl sulfoxide as a carrier. Compounds such as metal stearates (e.g., calcium stearate, zinc stearate, magnesium stearate, sodium stearate, lithium stearate, potassium stearate, etc.) can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant and as a delivery-enhancing or penetration-enhancing agent. Different salts, hydrates or solvates of the active ingredients can be used to further adjust the properties of the resulting composition.

In certain embodiments, one or both of the active agents as provided herein are administered parenterally, transdermally, mucosally, nasally, buccally, sublingualy, topically, or orally. In certain embodiments, the first active agent is administered orally in a tablet or capsule form. In certain embodiments, one or more of the active agents are administered topically (e.g. in the dosage form of a lotion or a liquid).

In certain embodiments, the composition provided herein may be applied in the form of a shampoo, foaming baths, spray, spot on, lotion, gels, emulsion, or other forms of application known to the person skilled in the art may also be used. A spray will mostly be used in curative application, whereas a shampoo mostly will have a cleaning and preventive function and a lotion is especially suitable for cleaning of exsudative lesions and ensures a major antiseptic action without distorting the microflora of the fur.

In certain embodiments, the compositions intended for pets, particularly cats and dogs, are generally applied by deposition on the skin (“spot on” or “pour on” application). This is generally a localized application to a region with a surface area of less than 10 cm², especially between 5 and 10 cm², in particular at two points and preferably localized between the animal's shoulders. After deposition, the composition diffuses, in particular over the animal's entire body, and then dries, without crystallizing or changing the appearance (in particular absence of any whitish deposit or of any dusty appearance) or the feel of the coat.

### 4.6.5. Kits

Active ingredients are often not administered to a patient at the same time or by the same route of administration. In some embodiments, provided herein are kits which, when used by the medical practitioner, can simplify the administration of appropriate amounts of active ingredients to a patient.

A typical kit comprises a unit dosage form of one of Compound A, Compound A1, Compound A2, Compound B, Compound B1, Compound C, or a pharmaceutically acceptable salt, stereoisomer, solvate, hydrate, clathrate, polymorph or prodrug thereof, and a unit dosage form of a second active ingredient, e.g. ivacaftor.

Kits can further comprise devices that are used to administer the active ingredient(s). Examples of such devices include, but are not limited to, syringes, drip bags, patches, and inhalers.

Kits can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### 5. EXAMPLES

Some embodiments provided herein are illustrated by the following non-limiting examples. The examples should not be construed as a limitation in the scope thereof.

### 5.1. Synthesis of 2-[1-(3-ethoxy-4-methoxyphenyl)-2-methylsulfonylethyl]-4-acetylaminoisoindoline-1,3-dione

A stirred solution of 1-(3-ethoxy-4-methoxyphenyl)-methylsulfonylethylamine (1.0 g, 3.7 mmol) and 3-acetamidophthalic anhydride (751 mg, 3.66 mmol) in acetic acid (20 mL) was heated at reflux for 15 h. The solvent was removed in vacuo to yield an oil. Chromatography of the resulting oil yielded the product as a yellow solid (1.0 g, 59% yield): mp, 144° C.; 1H NMR (CDCl₃) δ1.47 (t, J=7.0 Hz, 3H, CH₃), 2.26 (s, 3H, CH₃), 2.88 (s, 3H, CH₃), 3.75 (dd, J=4.4, 14.3 Hz, 1H, CHH), 3.85 (s, 3H, CH3), 4.11 (q, J=7 Hz, 2H, CH2), 5.87 (dd, J=4.3, 10.5 Hz, 1H, NCH), 6.82-6.86 (m, 1H, Ar), 7.09-7.11 (m, 2H, Ar), 7.47 (d, J=7 Hz, 1H, Ar), 7.64 (t, J=8 Hz, 1H, Ar), 8.74 (d, J=8 Hz, 1H, Ar), 9.49 (br s, 1H, NH); ¹³C NMR (CDCl₃) δ14.61, 24.85, 41.54, 48.44, 54.34, 55.85, 64.43, 111.37, 112.34, 115.04, 118.11, 120.21, 124.85, 129.17, 130.96, 136.01, 137.52, 148.54, 149.65, 167.38, 169.09, 169.40; Anal Calc'd. for C₂₂H₂₄NO₇S: C, 57.38; H, 5.25; N, 6.08. Found: C, 57.31; H, 5.34; N, 5.83.

### 5.2. Preparation of (+)-2-[1-(3-Ethoxy-4-methoxyphenyl)-2-methylsulfonylethyl]-4-acetylaminoisoindoline-1,3-dione (“Compound A1”)

### 5.2.1. Preparation of 3-Aminophthalic acid

A mixture of 10% Pd/C (2.5 g), 3-nitrophthalic acid (75.0 g, 355 mmol) and ethanol (1.5 L) was charged to a 2.5 L Parr hydrogenator, under a nitrogen atmosphere. Hydrogen was charged to the reaction vessel for up to 55 psi. The mixture was shaken for 13 hours, maintaining hydrogen pressure between 50 and 55 psi. Hydrogen was released and the mixture was purged with nitrogen 3 times. The suspension was filtered through a celite bed and rinsed with methanol. The filtrate was concentrated in vacuo. The resulting solid was reslurried in ether and isolated by vacuum filtration. The solid was dried in vacuo to a constant weight, affording 54 g (84% yield) of 3-aminopthalic acid as a yellow product. ¹H-NMR (DMSO-d6) δ: 3.17 (s, 2H), 6.67 (d, 1H), 6.82 (d, 1H), 7.17 (t, 1H), 8-10 (brs, 2H). ¹³C-NMR (DMSO-d6) δ: 112.00, 115.32, 118.20, 131.28, 135.86, 148.82, 169.15, 170.09.

### 5.2.2. Preparation of 3-acetamidophthalic anhydride

A 1 L 3-necked round bottom flask was equipped with a mechanical stirrer, thermometer, and condenser and charged with 3-aminophthalic acid (108 g, 596 mmol) and acetic anhydride (550 mL). The reaction mixture was heated to reflux for 3 hours and cooled to ambient temperature and further to 0-5° C. for another 1 hour. The crystalline solid was collected by vacuum filtration and washed with ether. The solid product was dried in vacuo at ambient temperature to a constant weight, giving 75 g (61% yield) of 3-acetamidopthalic anhydride as a white product. ¹H-NMR (CDCl₃) δ: 2.21 (s, 3H), 7.76 (d, 1H), 7.94 (t, 1H), 8.42 (d, 1H), 9.84 (s, 1H).

### 5.2.3. Resolution of 2-(3-ethoxy-4-methoxyphenyl)-1-(methylsulphonyl)-eth-2-ylamine

A 3 L 3-necked round bottom flask was equipped with a mechanical stirrer, thermometer, and condenser and charged with 2-(3-ethoxy-4-methoxyphenyl)-1-(methylsulphonyl)-eth-2-ylamine (137.0 g, 500 mmol), N-acetyl-L-leucine (52 g, 300 mmol), and methanol (1.0 L). The stirred slurry was heated to reflux for 1 hour. The stirred mixture was allowed to cool to ambient temperature and stirring was continued for another 3 hours at ambient temperature. The slurry was filtered and washed with methanol (250 L). The solid was air-dried and then dried in vacuo at ambient temperature to a constant weight, giving 109.5 g (98% yield) of the crude product (85.8% ee). The crude solid (55.0 g) and methanol (440 mL) were brought to reflux for 1 hour, cooled to room temperature and stirred for an additional 3 hours at ambient temperature. The slurry was filtered and the filter cake was washed with methanol (200 mL). The solid was air-dried and then dried in vacuo at 30° C. to a constant weight, yielding 49.6 g (90% recovery) of (S)-2-(3-ethoxy-4-methoxyphenyl)-1-(methylsulphonyl)-eth-2-ylamine-N-acetyl-L-leucine salt (98.4% ee). Chiral HPLC (1/99 EtOH/20 mM KH₂PO₄ @pH 7.0, Ultron Chiral ES-OVS from Agilent Technologies, 150 mm×4.6 mm, 0.5 mL/min., @240 nm): 18.4 min (S-isomer, 99.2%), 25.5 min (R-isomer, 0.8%).

### 5.2.4. Preparation of (+)-2-[1-(3-Ethoxy-4-methoxyphenyl)-2-methylsulfonylethyl]-4-acetylaminoisoindoline-1,3-dione

A 500 mL 3-necked round bottom flask was equipped with a mechanical stirrer, thermometer, and condenser. The reaction vessel was charged with (S)-2-(3-ethoxy-4-methoxyphenyl)-1-(methylsulphonyl)-eth-2-yl amine N-acetyl-L-leucine salt (25 g, 56 mmol, 98% ee), 3-acetamidophthalic anhydride (12.1 g 58.8 mmol), and glacial acetic acid (250 mL). The mixture was refluxed over night and then cooled to <50° C. The solvent was removed in vacuo, and the residue was dissolved in ethyl acetate. The resulting solution was washed with water (250 mL×2), saturated aqueous NaHCO₃ (250 mL×2), brine (250 mL×2), and dried over sodium sulphate. The solvent was evaporated in vacuo, and the residue recrystallized from a binary solvent containing ethanol (150 mL) and acetone (75 mL). The solid was isolated by vacuum filtration and washed with ethanol (100 mL×2). The product was dried in vacuo at 60° C. to a constant weight, affording 19.4 g (75% yield) of (S)-{2-[1-(3-ethoxy-4-methoxyphenyl)-2-methylsulfonylethyl]-4-inoisoindoline-1,3-dione with 98% ee. Chiral HPLC (15/85 EtOH/20 mM KH₂PO₄ @pH 0.5, Ultron Chiral ES-OVS from Agilent Technology, 150 mm×4.6 mm, 0.4 mL/min., @240 nm): 25.4 min (S-isomer, 98.7%), 29.5 min (R-isomer, 1.2%). ¹H-NMR (CDCl₃) δ:1.47 (t, 3H), 2.26 (s, 3H), 2.87 (s, 3H), 3.68-3.75 (dd, 1H), 3.85 (s, 3H), 4.07-4.15 (q, 2H), 4.51-4.61 (dd, 1H), 5.84-5.90 (dd, 1H), 6.82-8.77 (m, 6H), 9.46 (s, 1H). ¹³C-NMR (DMSO-d6) δ: 14.66, 24.92, 41.61, 48.53, 54.46, 55.91, 64.51, 111.44, 112.40, 115.10, 118.20, 120.28, 124.94, 129.22, 131.02, 136.09, 137.60, 148.62, 149.74, 167.46, 169.14, 169.48.

Specific crystalline forms of Compound A may be prepared according to U.S. Pat. No. 7,893,101, the disclosure of which is hereby incorporated by reference in its entirety.

### 5.3 Preparation of cyclopropanecarboxylic acid {2-[(1S)-1-(3-ethoxy-4-methoxy-phenyl)-2-methane-sulfonyl-ethyl]-3-oxo-2,3-dihydro-1H-isoindol-4-yl}-amide (“Compound B1”)

### 5.3.1. Preparation of Methyl 2-methyl-6-nitrobenzoate

A mixture of 2-methyl-6-nitrobenzoic acid (300.0 g, 1.66 moles, from Acros Organics, Morris Plains, N.J.) and trimethyl orthoacetate (298.3 g, 2.48 moles, from Aldrich Chemicals, Milwaukee, Wis.) was charged into a 3-L 3-necked flask at about 20-25° C. under nitrogen. The reaction mixture was gradually heated and the low-boiling point components generated during the reaction were distilled off to an internal temperature of 95-100° C. After 2 hours, the reaction mixture was cooled to 20-25° C. over 1-2 hours. After heptane (1.50 L, from Aldrich Chemicals) was charged into the reaction mixture over 1.0-1.5 hours, the reaction mixture was seeded with methyl 2-methyl-6-nitrobenzoate (0.5 g) when it became turbid. The suspension was cooled to 0-5° C. over 0.5-1 hour and kept at 0-5° C. for another 1.5-2 hours. The solid was collected by filtration under vacuum, washed with heptane (3×300 mL), and dried to a constant weight in a tray at 30-35° C. under a vacuum at 100-120 torr. The yield of methyl 2-methyl-6-nitrobenzoate was 292.0 g (91%), based on 300.0 g of 2-methyl-6-nitrobenzoic acid. The product was found to have a purity of >99% measured by HPLC based on area percentage, and a water content of <0.1% measured by Karl Fisher titration.

### 5.3.2. Preparation of Methyl 2-bromomethyl-6-nitrobenzoate

A mixture of methyl 2-methyl-6-nitrobenzoate (200.0 g, 1.02 moles, previously prepared), 1,3-dibromo-5,5-dimethylhydantoin (DBH, 162.0 g, 0.57 mole, from Aldrich Chemicals) and methyl acetate (1.20 L, from Aldrich Chemicals) was charged into a 3-L three-necked flask at about 20-25° C. under nitrogen. After the reaction mixture was refluxed for 0.5-1 hour, a solution of 2,2′-azobisisobutyronitrile (AIBN, 8.6 g, 52 mmol, from Aldrich Chemicals) in 100 mL of methyl acetate was charged over 15-30 minutes. The reaction mixture was refluxed for 6.5-8 hours until the amount of unreacted 2-methyl-6-nitrobenzoate was less than 5-10%. The reaction mixture was cooled to 15-18° C. and kept at 15-18° C. for 50-60 minutes. The solid was filtered, washed with cold (i.e., 5-10° C.) methyl acetate (2×100 mL) until there was less than 3% of methyl 2-bromomethyl-6-nitrobenzoate remained in the solid. Next, after heptane (1.00 L) was charged into the filtrate, the upper layer organic phase was washed with 2% of brine (2×500 mL) and deionized water (1-2×500 mL) until there was less than 0.5% (area percentage at 210 nm) of unreacted 5,5-dimethylhydantoin according to measurement by HPLC. After the solution was concentrated under a reduced pressure to remove about 1.80-1.90 L of methyl acetate, methyl tert-butyl ether (MTBE, 300 mL) was charged. After the reaction mixture was refluxed at 65-70° C. for 10-15 minutes, the solution was cooled to 50-55° C. over 0.5-1 hour and seeded with 500 mg of methyl 2-bromomethyl-6-nitrobenzoate at 45-50° C. The suspension was cooled to 20-25° C. and kept at 20-25° C. for 2-3 hours. The solids were collected by filtration, washed with 5-10° C. a cold mixture of heptane and MTBE in a volume ratio of 1:2 (2×100 mL), and dried to a constant weight at 20-25° C. under a vacuum at 100-120 torr. The yield of methyl 2-bromomethyl-6-nitrobenzoate was 185.2 g (66%), based on 200.0 g input of methyl 2-methyl-6-nitrobenzoate. The product was found to have a purity of >98% measured by HPLC based on area percentage, and a water content of <0.1% measured by Karl Fisher titration.

### 5.3.3. Preparation of (1S)-1-(3-ethoxy-4-methoxyphenyl)-2-methanesulfonyl-ethylamine

After a mixture of (1S)-1-(3-ethoxy-4-methoxyphenyl)-2-methanesulfonyl-ethylamine N-acetyl-L-Leucine salt (1.10 kg, 2.46 moles), deionized water (4.40 L), and dichloromethane (DCM, 5.50 L) was charged into a reaction vessel, a solution of sodium hydroxide (196.0 g, 4.90 moles) in 1.00 L of deionized water was charged into the reaction vessel over about 5 minutes at 15-25° C. The resulting mixture was stirred for at least 10 minutes at 15-25° C. and then the aqueous and organic phases were allowed to separate. The pH of the upper aqueous phase was maintained or adjusted at pH 13-14. The phases were separated and the upper aqueous phase was extracted with DCM (2×4.4 L). The pH of the aqueous phase was maintained at 13-14 throughout the extractions. The DCM extracts were combined and washed with deionized water (3.3 L) until the pH of the aqueous phase reached 11 or less. DCM was removed under vacuum below 35° C. The water content of the residual solid should be <0.1% w/w as measured by Karl Fisher titration. The residual solid was dried azeotropically with more DCM. The solid was dried to a constant weight in vacuo at 30-35° C. to give (1S)-1-(3-ethoxy-4-methoxyphenyl)-2-methanesulfonyl-ethylamine as a white powder (639.0-672.0 g, 95-100% yield).

### 5.3.4. Preparation of (1S)-7-nitro-2-[1-(3-ethoxy-4-methoxyphenyl)-2-(methylsulfonyl)ethyl]isoindolin-1-one

(1S)-7-nitro-2-[1-(3-ethoxy-4-methoxyphenyl)-2-(methylsulfonyl)-ethyl]isoindolin-1-one was prepared by the following procedure. A mixture of methyl 2-bromomethyl-6-nitrobenzoate (100.0 g, 365 mmol, prepared previously in Example 6.5.2.), (1S)-1-(3-ethoxy-4-methoxyphenyl)-2-methanesulfonylethylamine (104.7 g, 383 mmol, prepared previously in Example 6.5.3.), sodium hydrogen carbonate (67.5 g, 8.03 moles, from Aldrich Chemicals) and dimethyl formamide (500 mL) was charged into a 1-L 3-necked flask at room temperature under nitrogen. The reaction mixture was gradually heated to an internal temperature of 70-75° C. for two hours until there was less than <2% of unreacted methyl 2-bromomethyl-6-nitrobenzoate. The reaction mixture was gradually heated to an internal temperature of 95-100° C. for 18 hours. The reaction mixture was cooled to 20-25° C. and transferred to an 1-L addition funnel. After purified water (1500 mL) was charged into a 5-L 3-necked flask, the reaction mixture in the addition funnel was added into water in the 5-L 3-necked flask at room temperature over 1-2 hours maintaining an internal temperature below 30° C. The reaction mixture was stirred for 2 hours at room temperature. The solid was filtered out under vacuum, washed with water (3×300 mL) and methanol (2×400 mL), and then charged into a 2-L 3-necked flask followed by methanol (1000 mL). The mixture was refluxed for 1 hour. The mixture was cooled to room temperature. The solid was collected by filtration under vacuum, washed with 200 mL methanol (2 vol), and dried to a constant weight at 40-45° C. under a vacuum at 100-120 torr. The yield of (1S)-7-nitro-2-[1-(3-ethoxy-4-methoxyphenyl)-2-(methylsulfonyl)ethyl]isoindolin-1-one was 123.0 g (78%), based on 100.0 g input of methyl 2-bromomethyl-6-nitrobenzoate. The product was found to have a purity of >99% measured by HPLC based on area percentage, and a water content of <0.1% measured by Karl Fisher titration.

### 5.3.5. Alternative Preparation of (1S)-7-nitro-2-[1-(3-ethoxy-4-methoxyphenyl)-2-(methylsulfonyl)ethyl]isoindolin-1-one

(1S)-7-nitro-2-[1-(3-ethoxy-4-methoxyphenyl)-2-(methylsulfonyl)ethyl]isoindolin-1-one was also prepared by the following procedure. A mixture of methyl 2-bromomethyl-6-nitrobenzoate (100.0 g, 365 mmol, prepared previously in Example 6.5.2.), (1S)-1-(3-ethoxy-4-methoxyphenyl)-2-methanesulfonyl-ethylamine (104.7 g, 383 mmol, prepared previously in Example 6.5.3.), and potassium carbonate powder (100.8 g, 730 mmol, from Aldrich Chemicals) was suspended in acetonitrile (500 mL) at room temperature. The reaction mixture was refluxed at 81-83° C. for about two hours until there was less than 2% of unreacted methyl 2-bromomethyl-6-nitrobenzoate. After the reaction mixture was cooled to 45-50° C., methanol (200 mL) was charged over 5-10 minutes. After the mixture was allowed to cool to 20-25° C. and stirred for 2 hours, deionized water (1.40 L) was charged over 0.5-1 hour and stirred at 20-25° C. for 30 minutes and at 0-5° C. for 1-2 hours. The solid was filtered, washed with deionized water (3×300 mL), and dried to <10% of water content as measured by Karl Fisher titration. The solid was suspended in methanol (750 mL) and refluxed for 1-1.5 hours. The suspension was cooled to 0-5° C. over 1.5-2 hours and kept at 0-5° C. for 1-1.5 hours. The solid was filtered, washed with 0-5° C. methanol (2×200 mL) and heptane (200 mL), and then dried at 40-45° C. under vacuum to a constant weight. The yield of (1S)-7-nitro-2-[1-(3-ethoxy-4-methoxyphenyl)-2-(methylsulfonyl)ethyl]isoindolin-1-one was 148.0 g (93%), based on 100.0 g input of methyl 2-bromomethyl-6-nitrobenzoate. The product was found to have a purity of >99% measured by HPLC based on area percentage, and a water content of <1.0% measured by Karl Fisher titration.

### 5.3.6. Preparation of Compound B1

A mixture of (1S)-7-nitro-2-[1-(3-ethoxy-4-methoxyphenyl)-2-(methylsulfonyl)ethyl]isoindolin-1-one (60 g, 138 mmol, prepared previously in Example 6.5.5.), 10% Pd/C (50% wet, 2.4 g, 4 wt %, from Johnson Matthey, London, UK), ethyl acetate (780 mL) was charged into a Parr-vessel at room temperature under nitrogen. After the mixture was purged with nitrogen three times and with hydrogen three times, the reaction mixture was heated to 40° C. and then the heat was removed. The reaction mixture was stirred with hydrogen at a pressure between 40-45 psi over 4-6 hours until there was <3% of the hydroxylamine intermediate. The reaction mixture was cooled to 20-25° C. The reaction mixture was filtered through a celite bed (1 inch thickness) and then bed-washed with ethyl acetate (120 mL). The filtrate was transferred to a 3-L 3-necked flask equipped with a 50-mL addition funnel. After N,N-diisopropylethylamine (29 mL, 165 mmol) was charged into the flask, the addition funnel was charged with cyclopropylcarbonyl chloride (13.0 mL, 145 mmol, from Aldrich Chemicals). The cyclopropylcarbonyl chloride was added at room temperature over 1-2 hours at an internal temperature below 30° C. The reaction mixture was stirred for 2-4 hours at room temperature. After heptane (300 mL) was added, the reaction mixture was stirred for 4-6 hours. The solid was collected by filtration under vacuum, washed with 2N HCl (2×300 mL), water (2×300 mL) and then heptane (2×300 mL). The crude product was dried at 40-45° C. under a vacuum at 100-120 torr to a constant weight. The yield of crude Compound B1 was 58 g (88%), based on 60.0 g input of (1S)-7-nitro-2-[1-(3-ethoxy-4-methoxyphenyl)-2-(methylsulfonyl)ethyl]-isoindolin-1-one.

### 5.3.7. Recrystallization of Compound B1

A mixture of crude Compound B1 (95.2 g, prepared previously in Example 6.5.6.) and tetrahydrofuran (THF, 1.43 L) was charged into a 3 L flask at 20-25° C. under nitrogen. The suspension was heated to 60-65° C. until dissolution was achieved. The suspension was filtered at 45-50° C. and the solid was rinsed with 95 mL of THF prewarmed at 45-55° C. After about 950-1150 mL of THF was distilled off at normal pressure over 30-60 minutes, absolute ethanol (950 mL) was charged at 55-60° C. over 5-10 minutes. About 350-400 mL of solvents was removed at normal pressure until the internal temperature rose to 72-74° C. The resulting suspension was refluxed at 72-75° C. for 30-60 minutes, cooled to 20-25° C. over 1-2 hours and kept at 20-25° C. for another 1-2 hours. The solid was collected by filtration under vacuum, washed with absolute ethanol (240-280 mL) and heptane (240-280 mL), and then dried in tray at 50-55° C. in vacuo at 130-140 torr to a constant weight. The yield of the off-white crystalline product was (88.0-91.0 g, 92-96%).

The compounds described herein may also be prepared according to the process described in U.S. Patent Publication No. 2010/0168475, the disclosure of which is hereby incorporated by reference in its entirety.

### 5.4 Inhibition of PDE4

Phosphodiesterase 4 enzyme was purified from U937 human monocytic cells by gel filtration chromatography, and phosphodiesterase reactions were carried out as previously described. See, e.g., Muller et al., Bioorg. Med. Chem. Lett., 1998, 8(19): 2669-2674. Briefly, reactions were carried out in 96-well deep-well plates in 50 mM Tris HCl pH 7.5, 5 mM MgCl₂, 1 μM cyclic adenosine monophosphate (cAMP), plus 10 nM [³H]-cAMP for 45 min at 30° C. The reactions were terminated by boiling, treated with 1 mg/ml snake venom, and separated using AG-1X8 ion exchange resin (BioRad). Reactions consumed less than 15% of available substrate.

Enzyme assay data using purified PDE4 enzyme from U937 human monocytic cells indicate that Compound A1 has a PDE4 IC₅₀ of about 74 nM.

Enzyme assay data using purified PDE4 enzyme from U937 human monocytic cells indicate that Compound B1 has a PDE4 IC₅₀ of about 100 nM.

### 5.5 Inhibition of Foam Cell Formation

Formation of foam cells is a key step in the formation of atherosclerotic plaque. Thus, the efficacy of herein provided PDE4 modulators in inhibiting foam cell formation, and thus, treating, preventing and/or managing atherosclerosis, was investigated.

THP-1 cells in suspension were maintained in RPMI 1640 media (PenStep, 10% FBS, 37° C., 5% CO₂). The cells plated at 10⁵-10⁶ cells/well in 24-well plate (500 uL/well). Cell count was 34 at 1:20 and 1:30 dilution. Then, 100 nM PMA was added to allow for differentiation into macrophages, and the mixture was incubated for 24 hours. To the mixture, was added 500 U/mL IFN-gamma, and the resulting mixture was incubated for another 24 hours.

For vehicles, 50 μg/ml of acLDL and 500 U/ml IFN-γ were added to the cells. For those samples that received treatment by Compound A1, a first dose of 100 nM of Compound A1 was added 15 minutes before the addition of acLDL, and a second dose of 100 nM Compound A1 was added after 24 hours. Then, media was removed, and cells washed three times with PBS and fixed in 10% formalin for 10 minutes. Cells were rinsed in PBS (1 minute) and then with 60% isopropanol (15 seconds). Samples were stained with Oil Red O in darkness for 1 minute at 37° C. Samples were rinsed again in 60% isopropanol, then three times with PBS. Wells were kept hydrated until imaging, and samples were visualized using light microscopy.

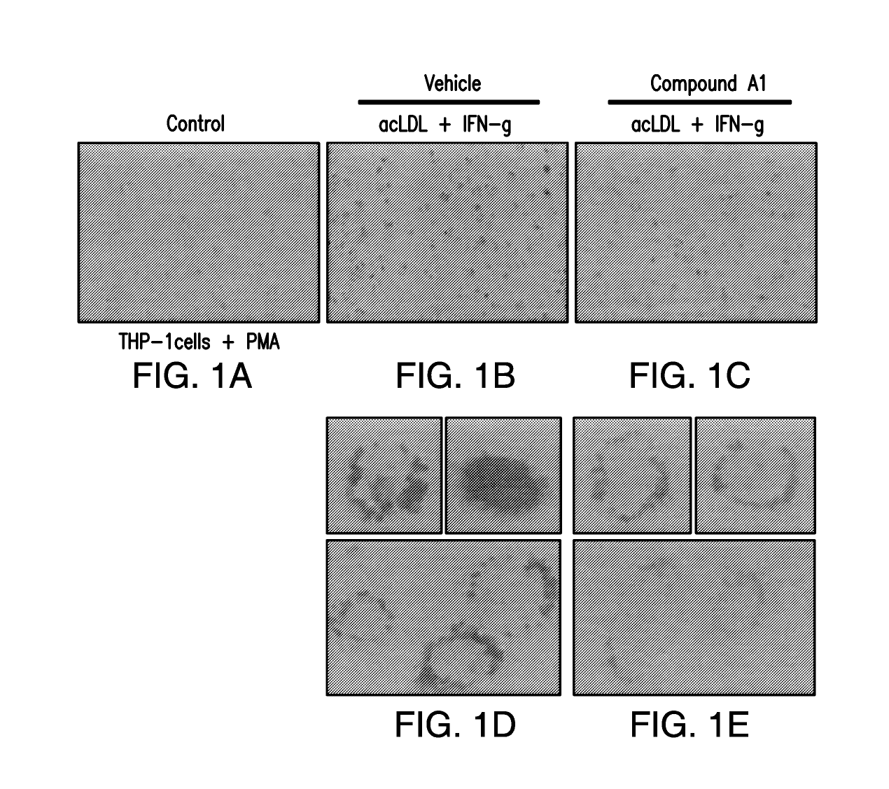

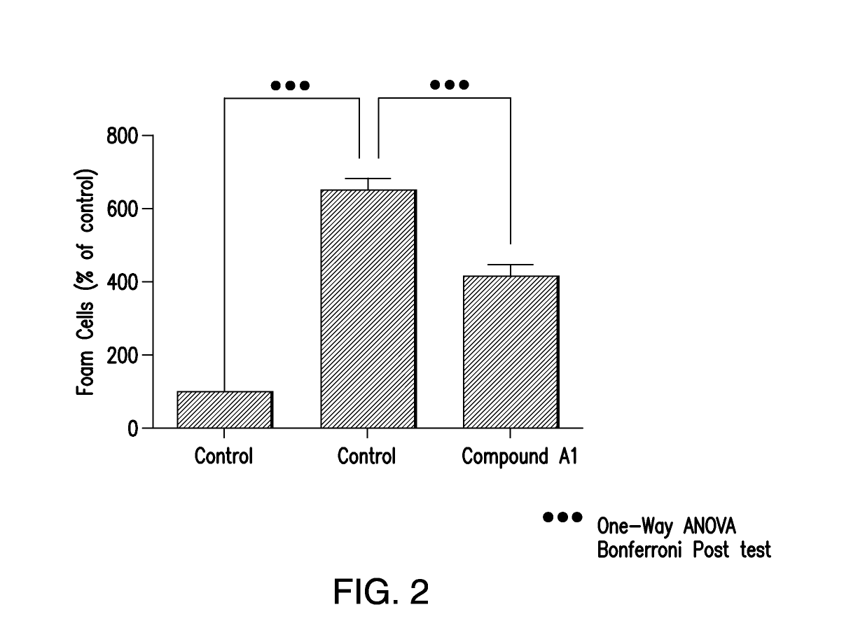

As shown in FIG. 1, treatment by Compound A1 (FIGS. 1C and 1E) resulted in significantly reduced formation of foam cells than the vehicle samples (FIGS. 1B and 1D). The results were normalized and plotted (FIG. 2). These results show that PDE4 modulators provided herein, in particular, Compound A2, are effective in preventing or inhibiting the formation of foam cells, which is a clear indication of the compounds' efficacy in treating, preventing, and/or managing atherosclerosis, or atherosclerotic cardiovascular disease.

The embodiments described above are intended merely to be exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

What is claimed is:

1. An effective amount of a compound being a PDE4 modulator for use in a method for treating, preventing or managing atherosclerosis, wherein the method comprises administering the compound to a subject in need thereof.

2. The compound for use of claim 1, wherein the compound is: 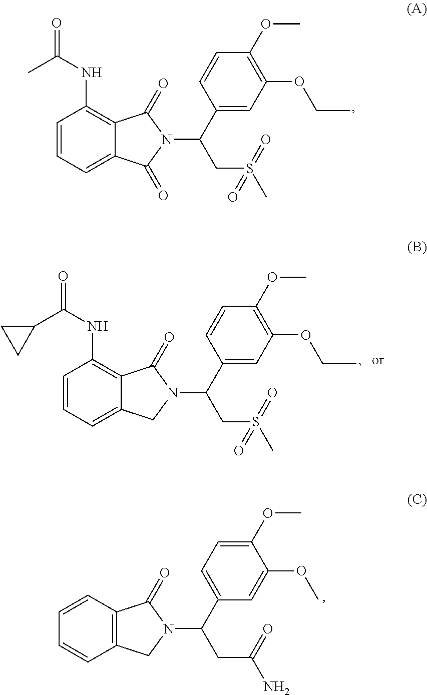 [SMILES: CCOc1cc(C(CS(C)(=O)=O)N2C(=O)c3cccc(NC(C)=O)c3C2=O)ccc1OC ; CCOc1cc(C(CS(C)(=O)=O)N2Cc3cccc(NC(=O)C4CC4)c3C2=O)ccc1OC ; COc1ccc(C(CC(N)=O)N2Cc3ccccc3C2=O)cc1OC]
or an enantiomer thereof, or a mixture of enantiomers thereof, or a pharmaceutically acceptable salt, solvate, hydrate, clathrate, or prodrug thereof.

3. The compound for use of claim 2, wherein the compound is 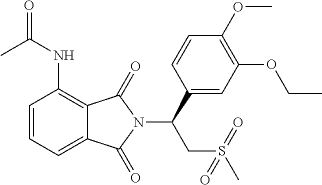 [SMILES: CCOc1cc([C@@H](CS(C)(=O)=O)N2C(=O)c3cccc(NC(C)=O)c3C2=O)ccc1OC]
or a pharmaceutically acceptable salt, solvate, hydrate, clathrate, or prodrug thereof; or wherein the compound is 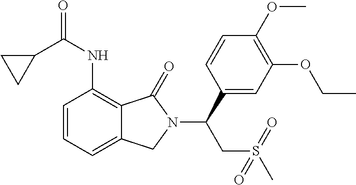 [SMILES: CCOc1cc([C@@H](CS(C)(=O)=O)N2Cc3cccc(NC(=O)C4CC4)c3C2=O)ccc1OC]
or a pharmaceutically acceptable salt, solvate, hydrate, clathrate, or prodrug thereof; or wherein the compound is 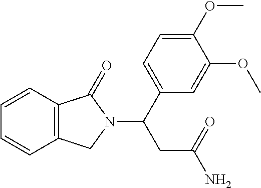 [SMILES: COc1ccc(C(CC(N)=O)N2Cc3ccccc3C2=O)cc1OC]
or a pharmaceutically acceptable salt, solvate, hydrate, clathrate, or prodrug thereof.

4. The compound for use of claim 1, wherein the subject is suffering from rheumatoid arthritis, psoriasis, or psoriatic arthritis.

5. An effective amount of a compound being a PDE4 modulator for use in a method for treating, preventing or managing atherosclerotic cardiovascular disease, wherein the method comprises administering the compound to a subject in need thereof.

6. The compound for use of claim 5, wherein the compound is: 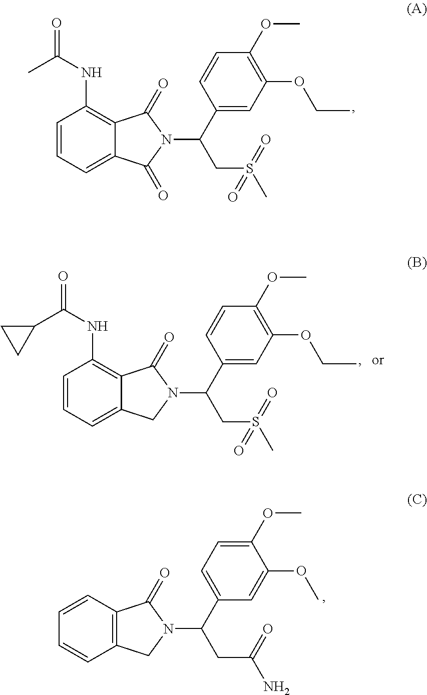 [SMILES: CCOc1cc(C(CS(C)(=O)=O)N2C(=O)c3cccc(NC(C)=O)c3C2=O)ccc1OC ; CCOc1cc(C(CS(C)(=O)=O)N2Cc3cccc(NC(=O)C4CC4)c3C2=O)ccc1OC ; COc1ccc(C(CC(N)=O)N2Cc3ccccc3C2=O)cc1OC]
or an enantiomer thereof, or a mixture of enantiomers thereof, or a pharmaceutically acceptable salt, solvate, hydrate, clathrate, or prodrug thereof.

7. The compound for use of claim 6, wherein the compound is 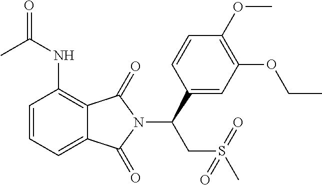 [SMILES: CCOc1cc([C@@H](CS(C)(=O)=O)N2C(=O)c3cccc(NC(C)=O)c3C2=O)ccc1OC]
or a pharmaceutically acceptable salt, solvate, hydrate, clathrate, or prodrug thereof; or wherein the compound is 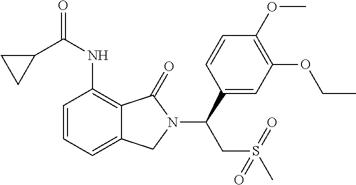 [SMILES: CCOc1cc([C@@H](CS(C)(=O)=O)N2Cc3cccc(NC(=O)C4CC4)c3C2=O)ccc1OC]
or a pharmaceutically acceptable salt, solvate, hydrate, clathrate, or prodrug thereof; or wherein the compound is 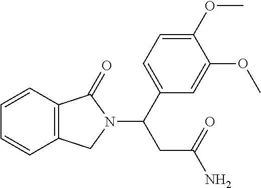 [SMILES: COc1ccc(C(CC(N)=O)N2Cc3ccccc3C2=O)cc1OC]
or a pharmaceutically acceptable salt, solvate, hydrate, clathrate, or prodrug thereof.

8. The compound for use of claim 5, wherein the subject is suffering from rheumatoid arthritis, psoriasis, or psoriatic arthritis.

9. The compound for use of claim 5, wherein the subject is suffering from rheumatoid arthritis.

10. The compound for use of claim 5, wherein the subject is suffering from psoriasis.

11. The compound for use of claim 5, wherein the subject is suffering from psoriatic arthritis.

12. An effective amount of a compound being a PDE4 modulator for use in a method for treating a symptom associated with atherosclerosis, wherein the method comprises administering the compound to a subject in need thereof.

13. The compound for use of claim 12, wherein the compound is: 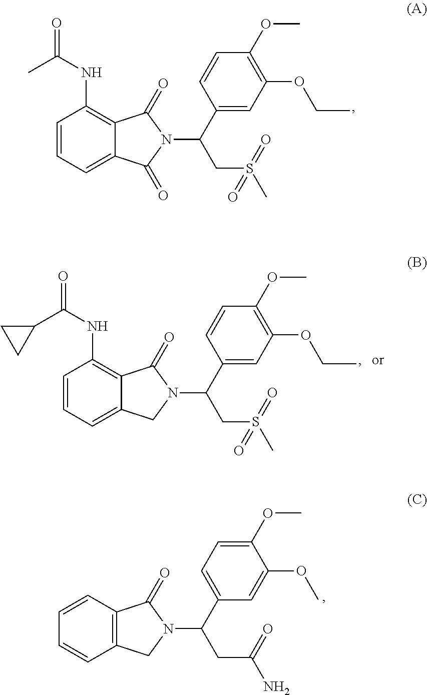 [SMILES: CCOc1cc(C(CS(C)(=O)=O)N2C(=O)c3cccc(NC(C)=O)c3C2=O)ccc1OC ; CCOc1cc(C(CS(C)(=O)=O)N2Cc3cccc(NC(=O)C4CC4)c3C2=O)ccc1OC ; COc1ccc(C(CC(N)=O)N2Cc3ccccc3C2=O)cc1OC]
or an enantiomer thereof, or a mixture of enantiomers thereof, or a pharmaceutically acceptable salt, solvate, hydrate, clathrate, or prodrug thereof.

14. The compound for use of claim 13, wherein the compound is 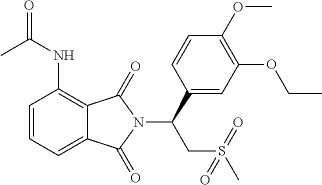 [SMILES: CCOc1cc([C@@H](CS(C)(=O)=O)N2C(=O)c3cccc(NC(C)=O)c3C2=O)ccc1OC]
or a pharmaceutically acceptable salt, solvate, hydrate, clathrate, or prodrug thereof; or wherein the compound is 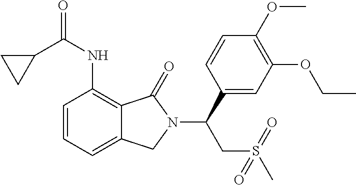 [SMILES: CCOc1cc([C@@H](CS(C)(=O)=O)N2Cc3cccc(NC(=O)C4CC4)c3C2=O)ccc1OC]
or a pharmaceutically acceptable salt, solvate, hydrate, clathrate, or prodrug thereof; or wherein the PDE4 modulator is 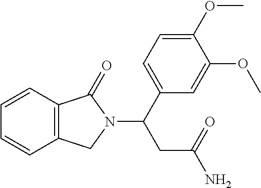 [SMILES: COc1ccc(C(CC(N)=O)N2Cc3ccccc3C2=O)cc1OC]
or a pharmaceutically acceptable salt, solvate, hydrate, clathrate, or prodrug thereof.

15. The compound for use of claim 12, wherein the subject is suffering from rheumatoid arthritis, psoriasis, or psoriatic arthritis.

16. The compound for use of claim 12, wherein the symptom is angina, shortness of breath, arrhythmia, a sleep problem, lack of energy, fatigue, sudden weakness, numbness, paralysis, trouble in seeing objects, dizziness, loss of consciousness, severe headache, or claudication.

17. The compound for use of any one of claims 1-16, wherein the method comprises oral administration of the compound.

18. The compound for use of claim 17, wherein the method comprises the administration of the compound in form of a tablet, capsule, or sachet.

19. The compound for use of any one of claims 1-16, wherein the method further comprises administering one or more additional active agents.
